(19) United States
(12) Patent Application Publication
(10) Pub. No.: US 2022/0125333 A1
(43) Pub. Date: Apr. 28, 2022

(54) MULTI-FUNCTION FACE MASKS

(71) Applicant: INNOVAPREP LLC, Drexel, MO (US)

(72) Inventors: David S. Alburty, Drexel, MO (US); Andew E. Page, Smithton, MO (US); John D. Birkenholz, Kansas City, MO (US); Kathleen Ruegsegger, Leawood, KS (US); Ann K. Packingham, Cleveland, MO (US); Zachary A. Packingham, Drexel, MO (US); Alec D. Adolphson, Raymore, MO (US); Pamela S. Murowchick, Lenexa, KS (US); Hugh Olsen, Davis, CA (US); Bryan C. Long, Odessa, MO (US); Steven D. Graham, Drexel, MO (US)

(21) Appl. No.: 17/511,487

(22) Filed: Oct. 26, 2021

Related U.S. Application Data
(60) Provisional application No. 63/141,951, filed on Jan. 26, 2021.
(60) Provisional application No. 63/105,847, filed on Oct. 26, 2020.

(51) Int. Cl. A61B 5/097; A61B 5/00

(52) U.S. Cl. CPC A61B 5/097; A61B 5/6803

(57) ABSTRACT

Devices and methods are described for using normal human breath to separately capture particles from inhaled and exhaled breath for analysis. This device can be constructed as a wearable device worn as a mask with separately removable filters on the inside and the outside of an efficient collection material.

This application claims priority to U.S. Provisional Patent Application Ser. No. 63/141,951, filed Jan. 26, 2021; and to U.S. Provisional Patent Application Ser. No. 63/105,847, filed Oct. 26, 2020, the contents of which are hereby incorporated by reference herein in their entirety into this disclosure.

### BACKGROUND OF THE SUBJECT DISCLOSURE

### Field of the Subject Disclosure

The subject disclosure relates generally to the field of microbiological sample collection and detection. More particularly, the subject disclosure relates to devices and methods for simultaneously monitoring human health and environmental health risks, while providing respiratory protection to the wearer.

### Background of the Subject Disclosure

The difficulties of detecting and quantifying dilute materials in air are well known. Detecting aerosol contaminants such as pathogens in breathing zones has previously relied on pumps or fans to collect particles on filters, in cyclones, impingers, or on impaction plates. They have been heavy, unwieldy and the sampling time has been limited by battery power or by tethering the subject to a fixed sampling device. This poses a significant problem to public health (SARS-CoV-2) and national security. For example, the postal anthrax attacks of 2001 and the subsequent war on terrorism, as well as natural pandemics such as MERS and SARS, have revealed shortcomings in the sampling and detection of biothreats. The medical arts are similarly affected by the existing limits of detection, as are the environmental sciences.

Existing samplers typically collect either the ambient air, or the breath; not both. Other breath collection devices are bulky and rely on an electromechanical vacuum to pull air from the breathing zone into a capture device.

Traditional flat filtration methodology is used to capture particles onto a flat filter, usually supported by a screen or fritted substrate. Many different methods of filtration exist, but all aim to attain the separation of two or more substances. This is achieved by some form of interaction between the substance or objects to be removed from, and trapped on, a filter. The substance that is to pass through the filter is a fluid, i.e., a liquid or gas. The simplest method of filtration is to pass a solution of a solid and fluid through a porous interface so that the solid is trapped, while the fluid passes through. This principle relies upon the size difference between the particles contained in the fluid, and the particles making up the solid. In the field, this is often done using a two-piece sealed filter holder with a removable filter.

Existing technologies include various masks and respirators that are only used to protect the individual from aerosols, and to prevent exhaled aerosols from the individual from contaminating or affecting others.

### SUMMARY OF THE SUBJECT DISCLOSURE

Existing masks and respirators do not provide for a way to extract or elute the sample from the mask, and do not separate the fraction of particles that would have been inhaled from the exterior environment from the particles in the exhaled breath.

A personal breath-driven sampling device system with removable filters for differential sample extraction would have significant applicability to pathogens that can be spread via aerosols, microdroplets, or droplets, such as SARS-CoV-2 (COVID-19) and others.

Devices as described can provide both personal protection from infection and protection of others from infectious individuals, while providing the opportunity to analyze and compare the samples from person to person and between the ambient environment and its inhabitants.

The present disclosure addresses the problem outlined and others, and advances the art by, for example, providing breath collection on a low pressure-drop electret filter that can be efficiently removed from the inside of a breath-driven sampling device (mask or respirator) and eluted to remove the particles for sample preparation and analysis.

### BRIEF DESCRIPTION OF THE DRAWINGS

FIG. 1 shows an exemplary embodiment of a protective mask with an opening to be covered by a collection filter assembly.

FIG. 2 shows an exemplary embodiment of a protective mask with two openings to be covered by two collection filter assemblies.

FIG. 3 shows an exemplary embodiment of a protective mask with an opening and border to be covered by a collection filter assembly.

FIG. 4 shows an exemplary embodiment of a protective mask with two openings with borders to be covered by two collection filter assemblies.

FIG. 5 shows an exemplary embodiment of a protective mask with a collection filter assembly.

FIG. 6 shows an exemplary embodiment of a protective mask with two collection filter assemblies.

FIGS. 7A and 7B show a protective mask with a two-layer collection filter assembly, according to an exemplary embodiment of the present subject disclosure.

FIGS. 8A and 8B show a protective mask with a three-layer collection filter assembly, according to an exemplary embodiment of the present subject disclosure.

FIGS. 9A and 9B show a protective mask with a four-layer collection filter assembly, according to an exemplary embodiment of the present subject disclosure.

FIG. 10 shows a protective mask with an alternative configuration of a collection filter assembly with a support frame, according to an exemplary embodiment of the present subject disclosure.

FIG. 11 shows a protective mask with an alternative configuration of two collection filter assemblies with support frames, according to an exemplary embodiment of the present subject disclosure.

FIGS. 12A and 12B show a protective mask with a two-layer collection filter assembly with support frame, according to an exemplary embodiment of the present subject disclosure.

FIGS. 13A and 13B show a protective mask with a three-layer collection filter assembly with support frame, according to an exemplary embodiment of the present subject disclosure.

FIGS. 14A and 14B show a protective mask with a four-layer collection filter assembly with support frame, according to an exemplary embodiment of the present subject disclosure.

FIGS. 15A and 15B show a protective mask with a five-layer collection filter assembly with support frame, according to an exemplary embodiment of the present subject disclosure.

FIGS. 16A and 16B show a protective mask with a two-layer collection filter assembly with a usage indicator, according to an exemplary embodiment of the present subject disclosure.

FIGS. 17A and 17B show a protective mask with a three-layer collection filter assembly with a usage indicator, according to an exemplary embodiment of the present subject disclosure.

FIGS. 18A and 18B show a protective mask with a four-layer collection filter assembly with a usage indicator, according to an exemplary embodiment of the present subject disclosure.

FIGS. 19A and 19B show a protective mask with a five-layer collection filter assembly with a usage indicator, according to an exemplary embodiment of the present subject disclosure.

FIG. 20 shows an exemplary embodiment of a protective mask with a cartridge-type collection filter assembly.

FIG. 21 shows an exemplary embodiment of a protective mask with two cartridge-type collection filter assemblies.

FIG. 22 shows an exemplary embodiment of an alternative protective mask design with a cartridge-type collection filter assembly.

FIG. 23 shows an exemplary embodiment of an alternative protective mask design with two cartridge-type collection filter assemblies.

FIG. 24 shows an exemplary embodiment of an alternative protective mask design with a cartridge-type collection filter assembly without a plastic cover.

FIG. 25 shows an exemplary embodiment of an alternative protective mask design with two cartridge-type collection filter assemblies without plastic covers.

FIG. 26 shows an exemplary embodiment of a cartridge-type collection filter assembly with a source and receptor filter.

FIG. 27 shows an exemplary embodiment of a cartridge-type collection filter assembly with a source filter and check valve.

FIG. 28 shows an exemplary embodiment of a cartridge-type collection filter assembly with a receptor filter and check valve.

FIG. 29 shows an exemplary embodiment of an alternative cartridge-type collection filter assembly with a source filter and check valve in a single assembly.

FIG. 30 shows an exemplary embodiment of a cartridge-type collection filter assembly in a centrifuge tube.

FIG. 31 shows an exemplary embodiment of an alternative cartridge-type collection filter assembly with a source filter and check valve in a centrifuge tube.

FIGS. 32A and 32B shows an exemplary embodiment of a cartridge-type collection filter assembly and a small specimen cup.

FIG. 33 shows an exemplary embodiment of an alternative cartridge-type collection filter assembly with a source filter and check valve in a small specimen cup.

FIGS. 34A and 34B show a protective mask with a pull-through, horizontal strip collection substrate, according to an exemplary embodiment of the present subject disclosure.

FIGS. 35A and 35B shows a protective mask with an alternative configuration of a pull-through, horizontal strip collection substrate with a welded attachment to the mask, according to an exemplary embodiment of the present subject disclosure.

FIGS. 36A and 36B shows a protective mask with a pull-through, vertical strip collection substrate with a welded attachment to the mask, according to an exemplary embodiment of the present subject disclosure.

FIG. 37 shows a protective mask with a horizontal strip collection substrate with a welded attachment to the mask, according to an exemplary embodiment of the present subject disclosure.

FIG. 38 shows a protective mask with an alternative configuration of a horizontal strip collection substrate without a welded attachment to the mask, according to an exemplary embodiment of the present subject disclosure.

FIG. 39 shows a protective mask with a vertical strip collection substrate with a welded attachment to the mask, according to an exemplary embodiment of the present subject disclosure.

FIG. 40 shows a protective mask with a pull-through, strip collection substrate on a plastic handle, according to an exemplary embodiment of the present subject disclosure.

FIG. 41 shows a protective mask with a pull-through, flat disk-shaped collection substrate on a plastic handle, according to an exemplary embodiment of the present subject disclosure.

FIG. 42 shows a protective mask with a pull-through, cylindrical collection substrate on a plastic handle, according to an exemplary embodiment of the present subject disclosure.

FIG. 43 shows a fold-flat protective mask with a pull-through, collection substrate on a plastic handle, according to an exemplary embodiment of the present subject disclosure.

FIG. 44 shows an alternative configuration of a fold-flat protective mask with a pull-through, collection substrate on a plastic handle, according to an exemplary embodiment of the present subject disclosure.

FIG. 45 shows a pull-through, strip collection substrate on a plastic handle, according to an exemplary embodiment of the present subject disclosure.

FIG. 46 shows a pull-through, flat disk-shaped collection substrate on a plastic handle, according to an exemplary embodiment of the present subject disclosure.

FIG. 47 shows a pull-through, cylindrical collection substrate on a plastic handle, according to an exemplary embodiment of the present subject disclosure.

FIG. 48 shows plastic adhesive sticker seal for a pull-through, collection substrate on a plastic handle, according to an exemplary embodiment of the present subject disclosure.

FIG. 49 provides an example table of how using source and receptor filters in series allows for data to be used to determine if the detected particles originated from the wearer or from the environment around the wearer.

FIG. 50 provides an example table of how using source and receptor filters in series allows for data to be used to determine if the detected particles originated from the wearer or from the environment around the wearer.

FIGS. 51A and 51B show a general design for a KN95 mask fitted with an inside and outside removable filter, according to an exemplary embodiment of the present subject disclosure.

FIG. 51C shows a frontal view of a mask with two filters, according to an exemplary embodiment of the present subject disclosure.

FIGS. 52A-52D show a procedure of removing two filters from a mask, according to an exemplary embodiment of the present subject disclosure.

FIGS. 53A-53C show an INNOVAPREP Bobcat Air Sampler flat filter device for eluting a removed filter into a sample cup using a charged foam canister, according to an exemplary embodiment of the present subject disclosure.

FIG. 54 shows virus presence using various filtration techniques.

FIGS. 55A-55B show requirements for a mask and ways to affix a filter to a mask, according to an exemplary embodiment of the present subject disclosure.

FIGS. 56A-56B show a tubular apparatus, according to an exemplary embodiment of the present subject disclosure.

FIG. 57 show a half face device with check valves integrated into the respirator or mask such that upon breathing in the user breathes through one receptor filter and upon breathing out the user breathes through a second, source filter, according to an exemplary embodiment of the present subject disclosure.

### DETAILED DESCRIPTION OF THE SUBJECT DISCLOSURE

The present disclosure provides a solution to the shortcomings of existing technologies, and describes novel devices and portable methods for simultaneous detection of, and monitoring for infective humans, and airborne pathogenic bioaerosols. The same device also serves as an infection control device for the wearer and those in the vicinity of the wearer. In some exemplary embodiments, this device is comprised of a wearable face mask with removable filter disks affixed to both the outside surface and inside surface of the mask. The “source” filter(s) on the inside of the mask collects respiratory pathogens an infective person can exhale while wearing the mask. The “receptor” filter(s) on the outside of the mask collects pathogenic aerosols that are deposited on the filter from inhaling while wearing the mask in a contaminated environment. This technology is suitable for both detection of aerosols and suspended droplets of pathogenic bacteria, fungi, and viruses, and non-infective aerosols and suspended droplets containing RNA, DNA, or cell fragment materials (originating from pathogens).

Ambient air may be collected on a low pressure-drop electret filter that can be efficiently removed from the outside face of the sampling device (mask or respirator) and eluted to remove the particles for sample preparation and analysis.

Exemplary devices according to the present disclosure can take the form of a mask or respirator, such as a common N95, KN95 respirator, or surgical mask. The mask is modified by adding a filter to the inside on one “hemisphere” (left or right side) of the mask and a corresponding filter on the outside on the opposite hemisphere. In this way, the protective features of the mask are not compromised. In fact, the added filters increase the collection efficiency of the mask. After being removed from the wearer, the filters are removed and placed into elution devices separately.

If the mask has high collection efficiency, such as N95, KN95, or surgical mask (up to 95% efficiency), the air composition difference between the inside and outside of the mask is inherently significant.

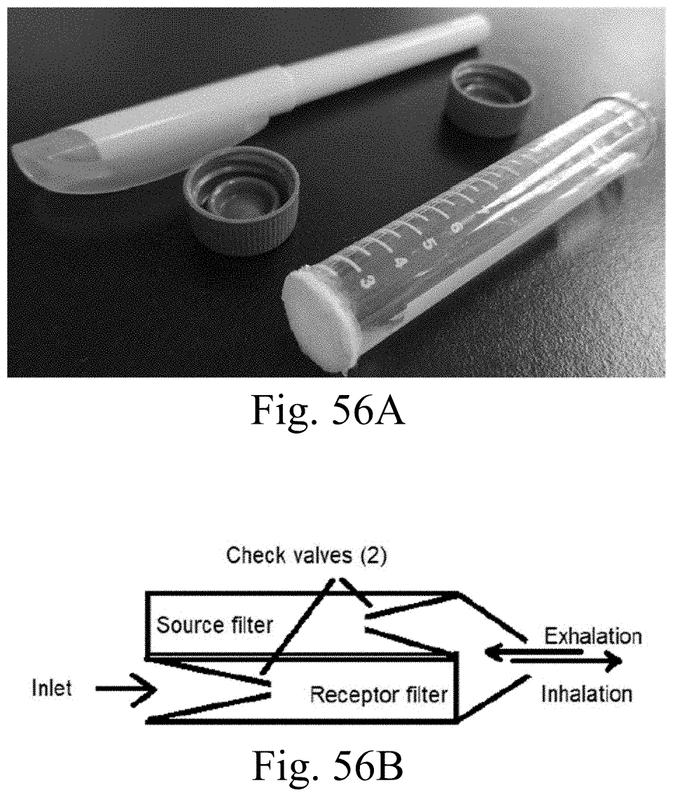

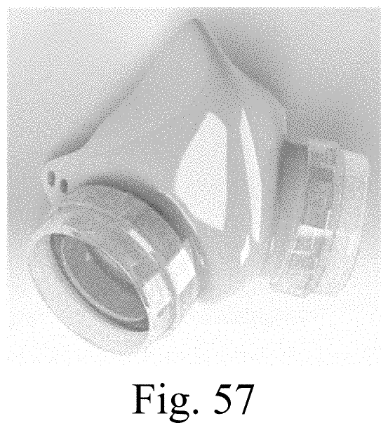

Although shown and described through this disclosure using a face mask as an example for sake of simplicity, the present disclosure also includes other forms, such as a tube that can be placed in the mouth or nostrils with two filters, one on the inhaling side, and one on the exhaling side using valves to regulate the flow of air. See FIGS. 56A-56B. As shown in FIG. 57, a half face mask having check valves such as shown for FIG. 56B is also possible, and within the purview of the present subject disclosure.

The present subject disclosure is a highly efficient filtration-based breath-powered sampling device for physical and biological surveillance. To operate the system, a new clean device is worn by the user for a period of time while breathing and during normal situational activities.

When the user has worn the device for the appropriate amount of time, the mask is removed and packaged for transport to a laboratory or analyzed onsite.

Due to the filtration efficiency of the mask material itself, the air composition difference between the inside filter and outside filter inherently differ if only the wearer is emitting particles (a source) or if the wearer is not emitting particles but is in a particle-laden environment (a receptor). Valves may be included in some exemplary embodiments.

As shown in experimental studies, about 9 minutes is needed to generate enough sample for PCR analysis. Given that sufficient numbers of virions may be collected in 9 minutes, smaller disks and multiple disks on a mask may be used. If the disks are on the inside, monitoring may be done for a period of several days (over the time someone typically wears the same mask). If the disks are on the outside, exposure may be conducted into as small as 4-6 hour intervals that, combined with contact tracing, would give a better idea of who is shedding a given virus. It's possible to repeatedly apply and collect sets of disks on the outside to monitor over several days. The disks could be a set in a strip to apply the single strip to the inside or outside of the mask but be able to collect the smaller disks independently from the strip when needed.

In the following figures, there will be shown and described exemplary configurations of disposable source/receptor sampling masks or respirators which may be used to simultaneously collect particles, including bioaerosols, from both the subject's environment and, separately, from the subject.

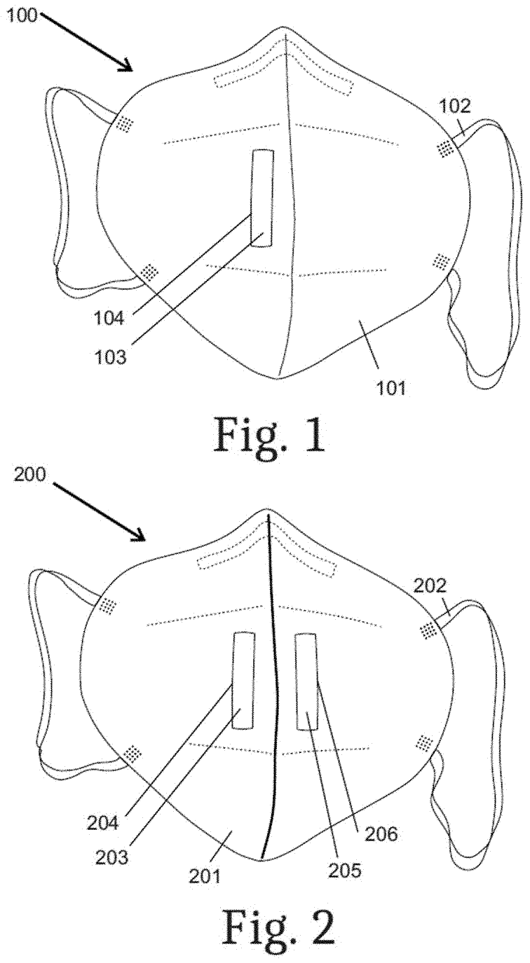

FIG. 1 shows an exemplary embodiment of a protective mask 100 with an opening 103 to be covered by a collection filter assembly. The mask 100 has a protective filter material 101 and includes a strap 102 for holding the mask 100 to the wearer's face by looping over the ears or around the back of neck and head. The mask 100 has an opening 103, with a perimeter 104, through the protective filter material 101. The opening 103 allows a collection filter assembly to be attached to the outside of the mask 100 with a bond sealing the collection filter assembly around the perimeter 104. Thus, when wearing the mask, exhaled and inhaled air can pass through the protective filter material 101 and through the opening 103 and the collection filter assembly. This allows for increased collection of target particles into the collection filter assembly while providing easy access to the collection filter assembly by health care workers. In one configuration of this embodiment, a protective cover web material may cover the opening 103 on the inside and/or the outside of the mask 100. This protective layer acts to provide structural integrity to the mask while only minimally increasing the pressure drop and minimally affecting the quantity of target particles passing through an attached collection filter assembly.

FIG. 2 shows an exemplary embodiment of a protective mask 200 with two openings 203 and 205 to be covered by a collection filter assembly. The mask 200 has a protective filter material 201 and includes a strap 202 for holding the mask to the wearer's face by looping over the ears or around the back of neck and head. The mask 200 has an opening 203, with a perimeter 204, and opening 205, with a perimeter 206, through the protective filter material 201. The opening 203 and opening 205 allow a collection filter assembly to be attached to the outside of the mask 200 with a bond sealing the collection filter assembly around the perimeter 204 and 206. Thus, when wearing the mask, exhaled and inhaled air can pass through the protective filter material 201 and through the openings 203 and 205 and the collection filter assembly over each opening. This allows for increased collection of target particles into the collection filter assembly while providing easy access to the collection filter assembly by health care workers. The use of two openings, covered with collection filter assemblies, is to allow a filter from one collection filter assembly to be used for a pooling sample and a filter from the other collection filter assembly to be used for an individual sample. Alternatively, one collection filter assembly can be configured for preferential “source sampling” and one collection filter assembly to be configured for preferential “receptor sampling.” Source sampling refers to sampling of target particles in the breath of the wearer of the mask, while receptor sampling refers to sampling of target particles in the environment around the mask wearer. In one configuration of this embodiment, a protective cover web material may cover the openings 203 and 205 on the inside and/or the outside of the mask 200. This protective layer acts to provide structural integrity to the mask while only minimally increasing the pressure drop and minimally affecting the quantity of target particles passing through an attached collection filter assembly.

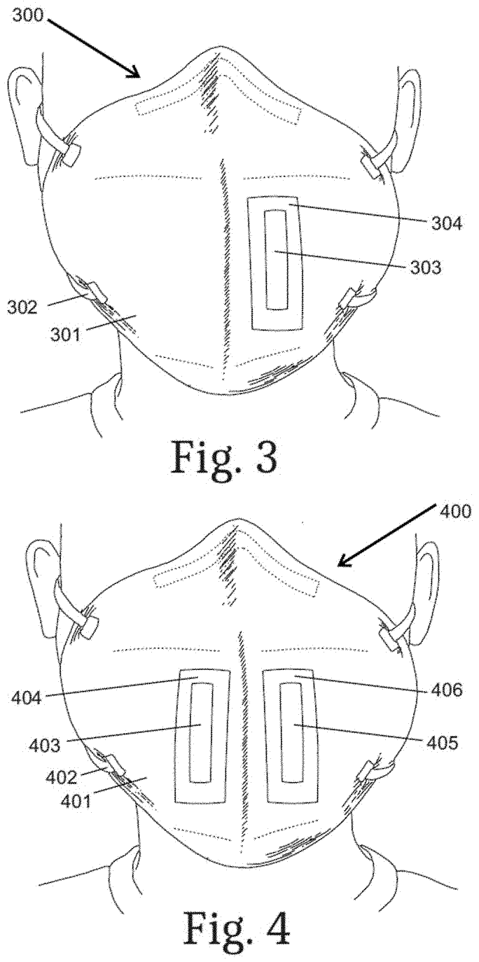

FIG. 3 shows an exemplary embodiment of a protective mask 300 with an opening 303 to be covered by a collection filter assembly. The mask 300 has a protective filter material 301 and includes a strap 302 for holding the mask 300 to the wearer's face by looping over the ears or around the back of neck and head. The mask 300 has an opening 303, with a perimeter 304, through the protective filter material 301. The opening 303 allows a collection filter assembly to be attached to the outside of the mask 300 with a bond sealing the collection filter assembly around the perimeter 304. Thus, when wearing the mask, exhaled and inhaled air can pass through the protective filter material 301 and through the opening 303 and the collection filter assembly. This allows for increased collection of target particles into the collection filter assembly while providing easy access to the collection filter assembly by health care workers. The mask 300 is similar to the mask 100 shown in FIG. 1. However, mask 300 has a wider and smoother perimeter around the opening 303 to enable the collection filter assembly to be more easily and securely bonded to the mask 300.

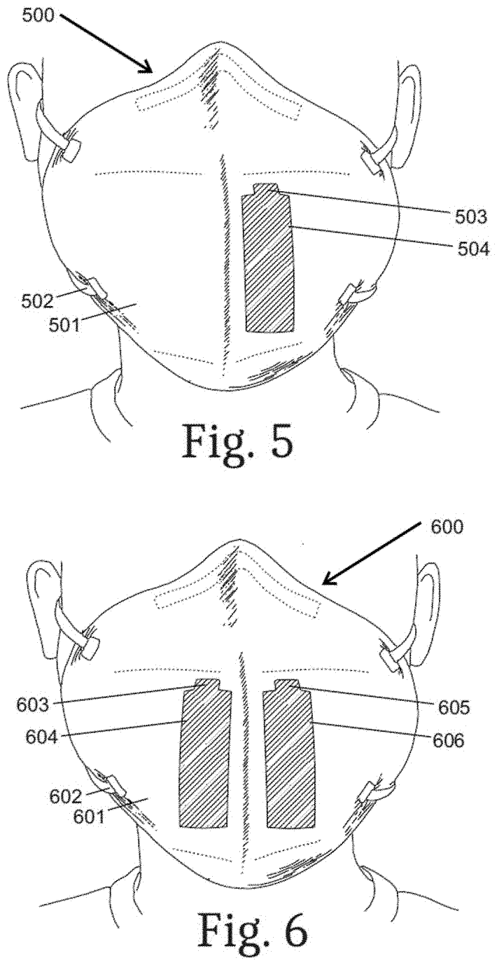

FIG. 4 shows an exemplary embodiment of a protective mask 400 with two openings 403 and 405 to be covered by a collection filter assembly. The mask 400 has a protective filter material 401 and includes a strap 402 for holding the mask to the wearer's face by looping over the ears or around the back of neck and head. The mask 400 has an opening 403, with a perimeter 404, and opening 405, with a perimeter 406, through the protective filter material 401. The opening 403 and opening 405 allow a collection filter assembly to be attached to the outside of the mask 400 with a bond sealing the collection filter assembly around the perimeter 404 and 406. Thus, when wearing the mask, exhaled and inhaled air can pass through the protective filter material 401 and through the openings 403 and 405 and the collection filter assembly over each opening. This allows for increased collection of target particles into the collection filter assembly while providing easy access to the collection filter assembly by health care workers. The use of two openings, covered with collection filter assemblies, is to allow a filter from one collection filter assembly to be used for a pooling sample and a filter from the other collection filter assembly to be used for an individual sample. Alternatively, one collection filter assembly can be configured for preferential “source sampling” and one collection filter assembly to be configured for preferential “receptor sampling.” Source sampling refers to sampling of target particles in the breath of the wearer of the mask, while receptor sampling refers to sampling of target particles in the environment around the mask wearer. The mask 400 is similar to the mask 200 shown in FIG. 2. However, mask 400 has a wider and smoother perimeter around the opening 403 and opening 405 to enable the collection filter assembly to be more easily and securely bonded to the mask 400.

FIG. 5 shows an exemplary embodiment of a protective mask 500 with a collection filter assembly 504. The mask 500 has a protective filter material 501 and includes a strap 502 for holding the mask 500 to the wearer's face by looping over the ears or around the back of neck and head. The mask 500 has an opening, with a perimeter, through the protective filter material 501. The opening, which is similar to opening 103 and opening 303 shown in FIG. 1 and FIG. 3, allows a collection filter assembly 504 to be attached to the outside of the mask 500 with a bond sealing the collection filter assembly around the perimeter of the opening.

Thus, when wearing the mask 500, exhaled and inhaled air can pass through the protective filter material 501 and through the opening in the protective filter material 501 and through the collection filter assembly 504. This allows for increased collection of target particles into the collection filter assembly while providing easy access to the collection filter assembly by health care workers. Tab 503 or collection filter assembly 504 allows for the health care worker, wearer of the mask, or other person to easily remove the collection filter assembly 504. Because collection filter assembly 504 can be easily removed from outside the mask, the potential for contamination of the health care worker or cross contamination of other samples is minimized. After removal of the collection filter assembly 504, target particles captured in the entire collection filter assembly may be extracted by placing the collection filter assembly 504 into a sample tube or other container and vortexing, shaking, sonicating, rocking, or with wet foam elution. Alternatively, dependent on the design of the collection filter assembly 504, the collection filter assembly 504 may be separated using tabs on the components and glove hands, forceps or other tools and collection filters within the assembly may be placed into sample container and shipped or transported to a lab for extraction, or extraction may be performed in the field.

It should be noted that the material in the collection filter assembly 504 has a particle removal efficiency similar to that of protective filter material 501 and a resistance (e.g., pressure drop) generally less than or equal to the resistance (e.g., pressure drop) of an equivalent surface area of protective filter material 501. In this way, the volume of air passing through the collection filter assembly 504 is similar to that passing through an equivalent surface area of protective filter material 501 and the amount of target captured per surface area is maximized.

FIG. 6 shows an exemplary embodiment of a protective mask 600 with two collection filter assemblies 604 and 606. This embodiment is similar to that shown in FIG. 5, but in the case of FIG. 6. the protective mask 600 contains two collection filter assemblies 604 and 606. The mask 600 has a protective filter material 601 and includes a strap 602 for holding the mask 600 to the wearer's face by looping over the ears or around the back of neck and head. The mask 600 has an opening, with a perimeter, through the protective filter material 601. The opening, which is similar to opening 103 and opening 303 shown in FIG. 1 and FIG. 3, respectively, allows a collection filter assembly 604 and collection filter assembly 606 to be attached to the outside of the mask 600 with a bond sealing the collection filter assemblies around the perimeter of the opening. Thus, when wearing the mask, exhaled and inhaled air can pass through the protective filter material 601 and through the openings in the protective filter material and through the collection filter assembly 604 and collection filter assembly 606. This allows for increased collection of target particles into the collection filter assembly while providing easy access to the collection filter assembly by health care workers.

Tab 603 on collection filter assembly 604 allows for the health care worker, wearer of the mask, or other person to easily remove the collection filter assembly 604. Because collection filter assembly 604 can be easily removed from outside the mask, the potential for contamination of the health care worker or cross contamination of other samples is minimized. Similarly, collection filter assembly 606 can be easily removed by grasping and peeling away tab 605. After removal of the collection filter assembly 604 and collection filter assembly 606, target particles captured in the entire collection filter assembly structure may be extracted by placing the collection filter assembly 604 and collection filter assembly 606 into a sample tube or other container and vortexing, shaking, sonicating, rocking, or with wet foam elution. Alternatively, dependent on the design of the collection filter assembly 604 and collection filter assembly 606, the collection filter assemblies may be separated using tabs on the components and gloved hands, forceps or other tools and collection filters within the assembly may be placed into sample container and shipped or transported to a lab for extraction or extraction may be performed in the field.

It should be noted that the material in collection filter assembly 604 and collection filter assembly 606 has a particle removal efficiency similar to that of protective filter material 601 and a resistance (e.g., pressure drop) generally less than or equal to the resistance (e.g., pressure drop) of an equivalent surface area of protective filter material 601. In this way, the volume of air passing through collection filter assembly 604 and collection filter assembly 606 is similar to that passing through an equivalent surface area of protective filter material 601 and the amount of target captured per surface area is maximized.

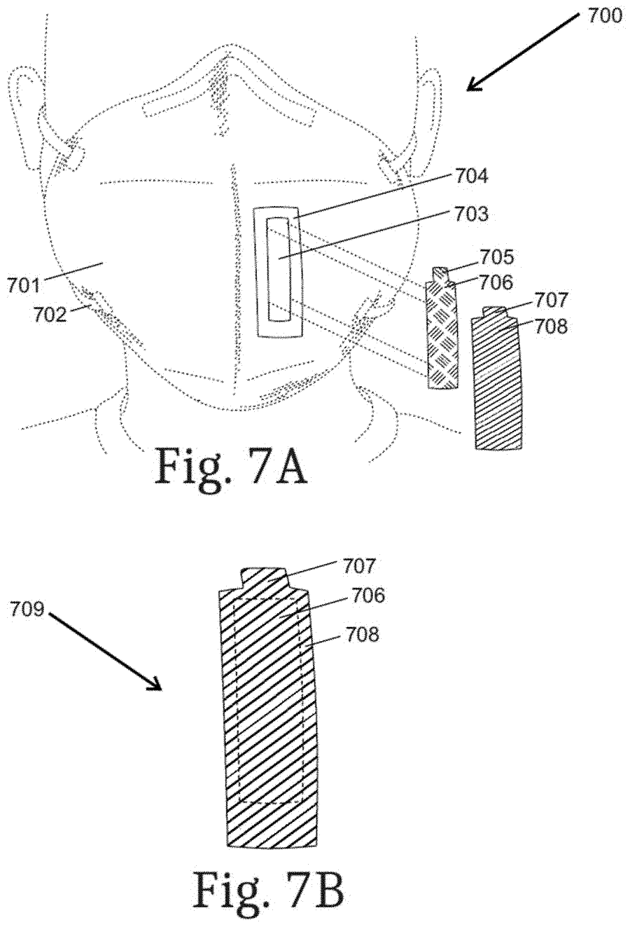

FIGS. 7A and 7B show a protective mask with a two-layer collection filter assembly, according to an exemplary embodiment of the present subject disclosure. The protective mask 700 has a protective filter material 701 and includes a strap 702 for holding the mask 700 to the wearer's face by looping over the ears or around the back of neck and head. The mask 700 has an opening 703, with a perimeter 704, through the protective filter material 701. The opening 703 allows a collection filter assembly 709 to be attached to the outside of mask 700 with a bond sealing the collection filter assembly around the perimeter of the opening.

Collection filter assembly 709 is made up of collection filter 706 with collection filter tab 705 and protective layer 708 with protective layer tab 707. Collection filter 706 is protected from contact, from outside the mask, by the protective layer 708.

When wearing the mask 700, exhaled and inhaled air can pass through the protective filter material 701 and through the opening 703 and collection filter assembly 709. In this exemplary embodiment, the resistance (e.g., pressure drop) for air flowing through the collection filter assembly 709 will generally be less than equal to the resistance (e.g., pressure drop) for air flowing through an equivalent surface area of the protective filter material 701. This configuration allows for increased airflow through the collection filter assembly 709 and therefor increased collection of target particles onto the collection filter assembly 709 while providing easy access to the collection filter assembly 709 by health care workers. The collection filter assembly 709 may be made up of all or a portion of the layers of material used in the protective filter material 701. In this embodiment specifically, the collection filter 706 maybe constructed from the internal filter material that is primary filter layer within the protective filter material 701 and the protective layer 708 may be constructed from the cover web that is outer layer often found within the protective filter material 701 of many N95 masks.

The collection filter 706 can be selected from a range of filter materials that will be known to those skilled in the art, including but not limited to: non-woven, woven, membrane, fiber, depth, electret, hydrophobic, or hydrophilic filtering materials. The protective layer 708 can be selected from a range of low resistance (e.g., pressure drop), open materials that offer protection from physical contact and provide structural integrity to the collection filter assembly 709, while limiting the potential for damage to the collection filter 706. These materials, that will be known to those skilled in the art, include but are not limited to: non-woven, woven, membrane, fiber, depth, hydrophobic, or hydrophilic materials.

The collection filter 706 and the protective layer 708 are bonded to each other to form the collection filter assembly 709. They may be bonded to each other using any of a number of approaches that will be known to those skilled in the art, including, but not limited to: adhesive, glue, tape, solvent bonding, ultrasonic weld, friction weld, laser welding, thermal bonding, or heat laminating. The bond between the layers may be permanent, wherein the entire collection filter assembly 709 will be removed from the mask 700 for placement into a sample tube for particle extraction, or the bond between the layers may be removable, or they may be bonded with a tear-away portion, to allow for separation of the layers. In this way, the protective layer 708 may be discarded and the collection filter 706 may be placed into a sample tube for extraction.

Similarly, the collection filter assembly 709 is then bonded to the border 704 using any of a number of approaches that will be known to those skilled in the art, including, but not limited to: adhesive, glue, tape, solvent bonding, ultrasonic weld, friction weld, laser welding, thermal bonding, or heat laminating. The bond between the collection filter assembly 709 and the border 704 must be airtight and removable. In this way, when worn, a large amount of exhaled and inhaled air will pass through the collection filter assembly 709 layers and when the sampling period is complete the entire collection filter assembly may be easily removed from the mask 700.

Tab 703 of collection filter assembly 709 allows for the health care worker, wearer of the mask, or other person to easily remove the collection filter assembly 709 by grasping collection filter tab 705, or protective layer tab 707, or both together. The person removing the collection filter assembly 709 will generally be expected to have gloved hands and will place one hand onto the top of the nose area of the mask 700 and grasp the collection filter tab 705 and protective layer tab 707 with the other hand and pull down to remove the collection filter assembly 709. Other means of removing the collection filter assembly 709, such as pulling up on the collection filter tab 705 and protective layer tab 707, with the tabs at the bottom of the collection filter assembly 709, are also possible and will be apparent to those known in the art. Because collection filter assembly 709 can be easily removed from outside the mask, the potential for contamination of the health care worker or cross contamination of other samples is minimized.

After removal of the collection filter assembly 709, target particles captured in the entire collection filter assembly may be extracted by placing the collection filter assembly 709 into a sample tube or other container and vortexing, shaking, sonicating, rocking, or with wet foam elution. Alternatively, dependent on the design of the collection filter assembly 704, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 704 has a particle removal efficiency similar to that of protective filter material 701 and a resistance (e.g., pressure drop) generally less than or equal to the resistance (e.g., pressure drop) of an equivalent surface area of protective filter material 701. In this way, the volume of air passing through the collection filter assembly 704 is similar to that passing through an equivalent surface area of protective filter material 701 and the amount of target captured per surface area is maximized.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature. Additionally, human DNA targets may be analyzed for using polymerase chain reaction, sequencing or other methods that will be well known in the art. The human DNA target results can then be used to determine the approximate wear time for the mask and correlate this back to the exposure amount or amount of virus, microbial or other target being shed by the wearer.

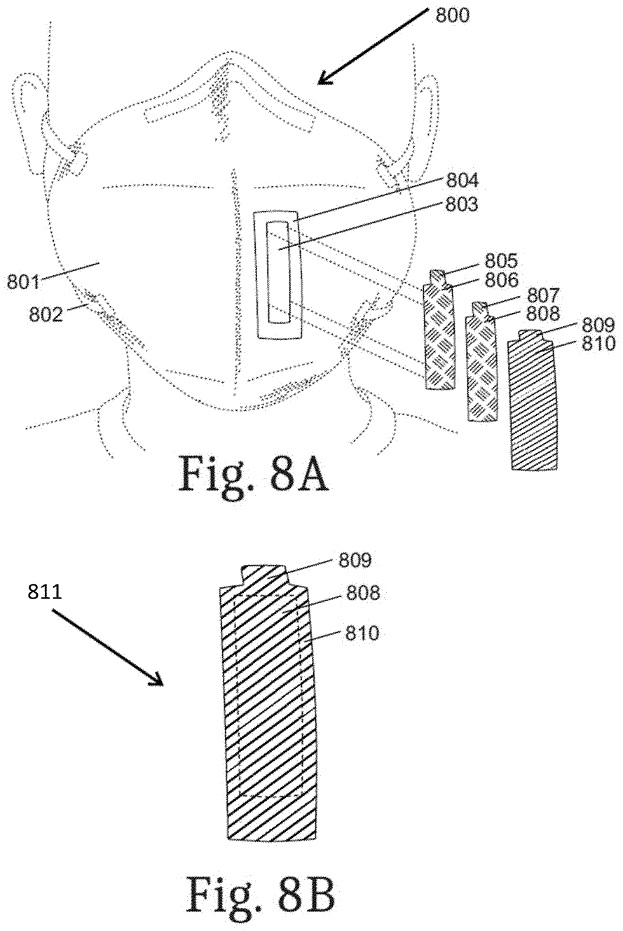

FIGS. 8A and 8B show a protective mask with a three-layer collection filter assembly, according to an exemplary embodiment of the present subject disclosure. The protective mask 800 has a protective filter material 801 and includes a strap 802 for holding the mask 800 to the wearer's face by looping over the ears or around the back of neck and head. The mask 800 has an opening 803, with a perimeter 804, through the protective filter material 801. The opening 803 allows a collection filter assembly 811 to be attached to the outside of mask 800 with a bond sealing the collection filter assembly around the perimeter of the opening.

Collection filter assembly 811 is made up of three layers of material. Moving from the interior face to the exterior face the layers are a source collection filter 806 with a source collection filter tab 805, a receptor collection filter 808 with a receptor collection filter tab 807, and a protective layer 810 with protective layer tab 809. Source collection filter 806 receives significant protection from particle exposure during inhalation by receptor collection filter 808 and, similarly, receptor collection filter 808 receives significant protection from particle exposure during exhalation by source collection filter 806. Further, receptor collection filter 808 is protected from contact during mask use and during removal of the collection filter assembly 811 for processing, by the protective layer 810.

The described configuration allows source collection filter 806 to be analyzed for evidence of the wearer being a carrier or source of viral or microbial aerosols. Receptor collection filter 808 can similarly be analyzed, but for evidence of the wearer potentially being exposed, or in an exposing environment, or in close proximity to a carrier. The combined efficiency of source collection filter 806 and receptor collection filter 808 will generally be similar to that of the protective filter material 801. With approximately 50% of the combined efficiency coming from source collection filter 806 and 50% coming from receptor collection filter 808. In one preferred embodiment, the collection filter assembly 811 is constructed from the same materials and in the same order that the protective filter material 801 is constructed. In this case, the collection filter assembly 811 can even be constructed from the material cut from opening 803 with a thin non-porous border added to the material or to the perimeter 804 to allow the collection filter assembly 811 to overlap and form an air-tight seal with mask 800.

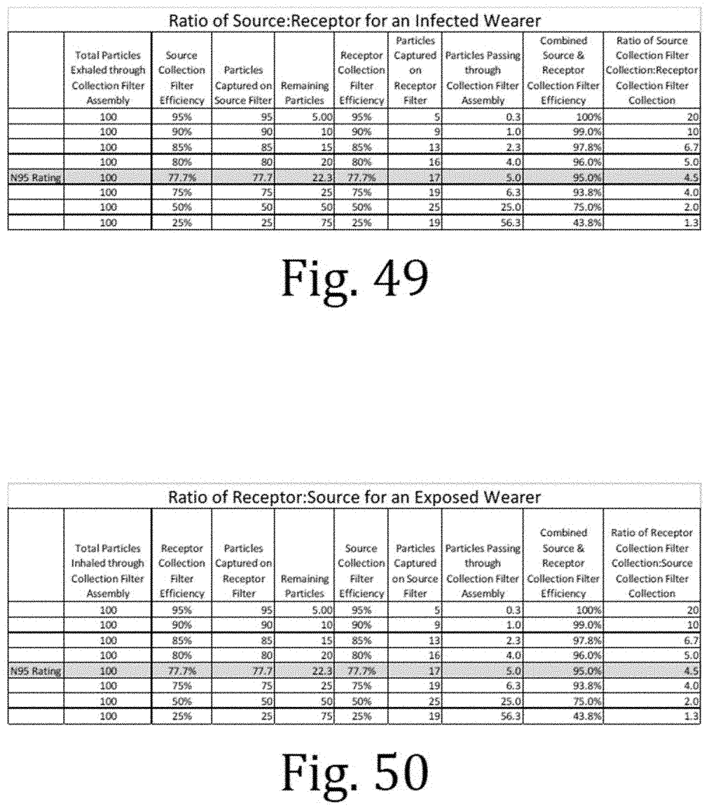

N95 and KN95 masks are rated to capture a minimum of 95% of 0.3 μm particles. N95 and KN95 masks are often manufactured with a 4-layer construction consisting of two identical filter layers sandwiched between two identical cover web layers. The filter layers are more susceptible to damage that could reduce the efficiency rating and the stronger cover web material is used to provide integrity to the mask and protection to the filter layers. In this case, each filter layer must provide a minimum of approximately 77.7% efficiency for 0.3 μm particles for the combined efficiency of the two layers to reach 95%. The tables provided in FIG. 49 and FIG. 50 provide an example of how using source and receptor filters in series, as was described in the previous paragraph, allows for data to be used to determine if the detected particles originated from the wearer or from the environment around the wearer. Assuming a 77.7% efficiency for each filter layer, and in the case of an infected wearer that is shedding virus, the inside source collection filter 806 will collect a minimum of approximately 4.5 times more target particles than the receptor collection filter 808. This difference is sufficient to allow for a determination as to whether target particles originated from an infected wearer or a contaminated environment. Further, as the efficiency of each layer is increased the ratio of target particles collected by each filter increases. As an example, two 90% efficiency filters will provide a minimum of approximately 10 times more particles on source collection filter 806 if the wearer is contaminated or 10 times more on receptor collection filter 808 if the wearer is in a contaminated environment. Finally, these estimated ratios are most likely lower than they will be in actual use cases because not all particles containing target material are 0.3 μm in diameter, but are rather distributed over a large particle size range, and, in general, particles larger than and smaller than 0.3 μm in diameter are captured with higher efficiency which will lead to larger ratios.

When wearing the mask 800, exhaled and inhaled air can pass through the protective filter material 801 and through the opening 803 and collection filter assembly 811. In this exemplary embodiment, the resistance (e.g., pressure drop) for air flowing through the collection filter assembly 811 will generally be less than or equal to the resistance (e.g., pressure drop) for air flowing through an equivalent surface area of the protective filter material 801. This configuration allows for increased airflow through the collection filter assembly 811 and therefor increased collection of target particles onto the collection filter assembly 811 while providing easy access to the collection filter assembly 811 by health care workers that are processing samples. The collection filter assembly 811 may be made up of all or a portion of the layers of material used in the protective filter material 801. In this embodiment specifically, the source collection filter 806 and receptor collection filter 808 may be constructed from the internal filter material within the protective filter material 801 and the protective layer 810 may be constructed from the cover web that is the outer layer often found within the protective filter material 801 of many N95 masks.

The source collection filter 806 and receptor collection filter 808 can be selected from a range of filter materials that will be known to those skilled in the art, including but not limited to: non-woven, woven, membrane, fiber, depth, electret, hydrophobic, or hydrophilic filtering materials. The protective layer 810 can be selected from a range of low resistance (e.g., low pressure drop) materials that offer protection from physical contact and provide structural integrity to the collection filter assembly 811, while limiting the potential for damage to source collection filter 806 and receptor collection filter 808. These materials, that will be known to those skilled in the art, including but not limited to: non-woven, woven, membrane, fiber, depth, hydrophobic, or hydrophilic materials.

Source collection filter 806, receptor collection filter 808, and protective layer 810 are bonded to each other to form the collection filter assembly 811. They may be bonded to each other using any of a number of approaches that will be known to those skilled in the art, including, but not limited to: adhesive, glue, tape, solvent bonding, ultrasonic weld, friction weld, laser welding, thermal bonding, or heat laminating. The bond between the layers may be permanent, wherein the entire collection filter assembly 811 will be removed from the mask 800 for placement into a sample tube for particle extraction, or the bond between the layers may be removable, or they may be bonded with a tear-away portion, to allow for separation of the layers. In this way, the protective layer 810 may be discarded and the source collection filter 806 and receptor collection filter 808 may be placed into a single sample tube or two separate sample tubes for shipment to a lab or for immediate extraction. Further, tamper evident seals may be used on the collection filter assembly 811, to ensure that it was not removed or tampered with prior to the final removal and analysis.

Similarly, the collection filter assembly 811 is then bonded to the border 804 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 811 and the border 804 must be airtight and removable. In this way, when worn, a large amount of exhaled and inhaled air will pass through the collection filter assembly 811 layers and when the sampling period is complete the entire collection filter assembly may be easily removed from the mask 800.

Tabs on the collection filter assembly 811 allow for the health care worker, wearer of the mask, or another person to easily remove the collection filter assembly 811. By grasping source collection filter tab 805, receptor collection filter tab 807, or protective layer tab 809, or a combination of the tabs together the worker or wearer can easily remove the collection filter assembly 811. The collection filter assembly 811 may be designed such that the source collection filter tab 805 is the largest of the tabs and can be easily grabbed by itself or the three tabs may be bonded partially together to form a single tab that is grasped for removal. Finally, the source collection filter 806, receptor collection filter 808, and protective layer 810 can have a single combined tab that is formed by bonding a portion of the tab area together, but with a perforation that allows the top of the tab to be torn off revealing separate tabs on each layer that can be used for separation of the layers.

The described approach allows collection filter assembly 811 to be removed from mask 800 without having to remove mask 800. The person removing the collection filter assembly 811 will generally be expected to have gloved hands and will place one hand onto the outside of mask 800 near the nose area and grasp the source collection filter tab 805, receptor collection filter tab 807, and protective layer tab 809 with the other hand and pull down to remove the collection filter assembly 811. Other means of removing the collection filter assembly 811, such as pulling up on the source collection filter tab 805, receptor collection filter tab 807, and protective layer tab 809, with the tabs at the bottom of the collection filter assembly 811, will be apparent to those known in the art. Because collection filter assembly 811 can be easily removed from outside the mask, the potential for contamination of the health care worker or cross contamination of other samples is minimized. After removal of the collection filter assembly 811, opening 803 can be covered by placing a new collection filter assembly 811 over the opening or by placing a temporary pressure sensitive adhesive seal over the opening. In this way, it is possible for a health care worker to collect samples from a large number of people in a short period of time with minimal potential for exposure to viral or microbial aerosols. Alternatively, for home or non-health care facility use, it is possible for the wearer to remove mask 800 and then remove collection filter assembly 811 from mask 800.

After removal of collection filter assembly 811, target particles captured in the entire collection filter assembly 811 may be extracted by placing the collection filter assembly 811 into a sample tube or other container and vortexing, shaking, sonicating, rocking, or with wet foam elution. Alternatively, dependent on the design of the collection filter assembly 811, it may be separated using tabs on the components and gloved hands, forceps or other tools and collection filters within the assembly may be placed into a sample container and shipped or transported to a lab for extraction or extraction may be performed in the field.

It should be noted that the material in the collection filter assembly 811 has a particle removal efficiency similar to that of protective filter material 801 and a resistance (e.g., pressure drop) generally less than or equal to the resistance (e.g., pressure drop) of an equivalent surface area of protective filter material 801. In this way, the volume of air passing through the collection filter assembly 811 is similar to that passing through an equivalent surface area of protective filter material 801 and the amount of target captured per surface area is maximized.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

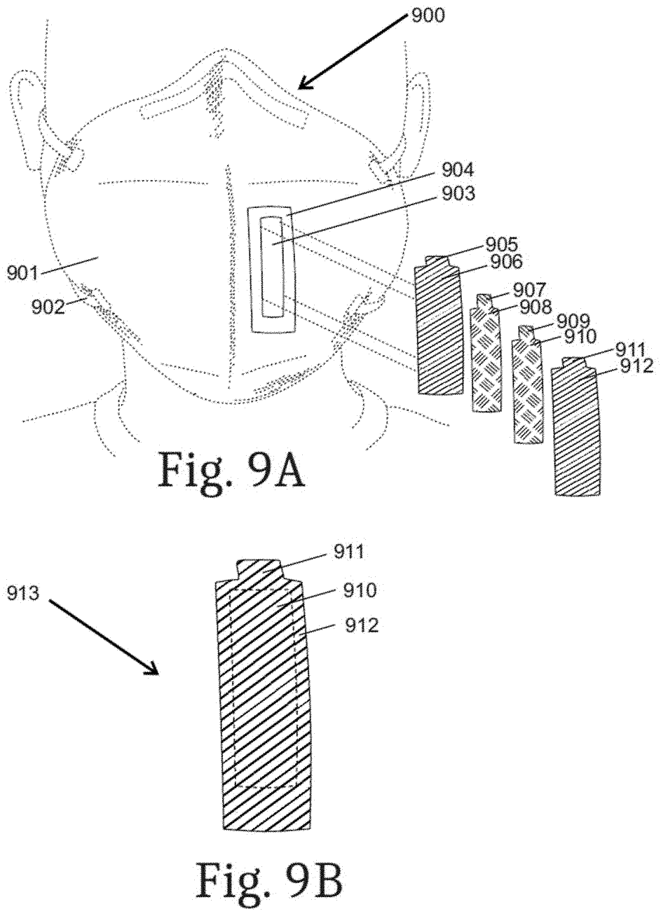

FIGS. 9A and 9B show a protective mask with a four-layer collection filter assembly, according to an exemplary embodiment of the present subject disclosure. The protective mask 900 has a protective filter material 901 and includes a strap 902 for holding the mask 900 to the wearer's face by looping over the ears or around the back of neck and head. The mask 900 has an opening 903, with a perimeter 904, through the protective filter material 901. The opening 903 allows a collection filter assembly 913 to be attached to the outside of mask 900 with a bond sealing the collection filter assembly around the perimeter of the opening.

Collection filter assembly 913 is made up of four layers of material. Moving from the interior face to the exterior face the layers are an interior protective layer 906 with interior protective layer tab 905, source collection filter 908 with a source collection filter tab 907, a receptor collection filter 910 with a receptor collection filter tab 909, and an exterior protective layer 912 with exterior protective layer tab 911. Source collection filter 908 receives significant protection from particle exposure during inhalation by receptor collection filter 910 and, similarly, receptor collection filter 910 receives significant protection from particle exposure during exhalation by source collection filter 908. Further, receptor collection filter 910 is protected from contact during mask use and during removal of the collection filter assembly 913 for processing, by the protective layer 912.

The described configuration allows source collection filter 908 to be analyzed for evidence of the wearer being a carrier or source of viral or microbial aerosols. Receptor collection filter 910 can similarly be analyzed, but for evidence of the wearer potentially being exposed, or in an exposing environment, or in close proximity to a carrier. The combined efficiency of source collection filter 908 and receptor collection filter 910 will generally be similar to that of the protective filter material 901. With approximately 50% of the combined efficiency coming from source collection filter 908 and 50% coming from receptor collection filter 910. In one preferred embodiment, the collection filter assembly 913 is constructed from the same materials and in the same order that the protective filter material 901 is constructed. In this case, the collection filter assembly 913 can even be constructed from the material cut from opening 903 with a thin non-porous border added to the material or to the perimeter 904 to allow the collection filter assembly 913 to overlap and form an air-tight seal with mask 900.

N95 and KN95 masks are rated to capture a minimum of 95% of 0.3 μm particles. N95 and KN95 masks are often manufactured with a 4-layer construction consisting of two identical filter layers sandwiched between two identical cover web layers. The filter layers are more susceptible to damage that could reduce the efficiency rating and the stronger cover web material is used to provide integrity to the mask and protection to the filter layers. In this case, each filter layer must provide a minimum of approximately 77.7% efficiency for 0.3 μm particles for the combined efficiency of the two layers to reach 95%. The tables provided in FIG. 49 and FIG. 50 provide an example of how using source and receptor filters in series, as was described in the previous paragraph, allows for data to be used to determine if the detected particles originated from the wearer or from the environment around the wearer. Assuming a 77.7% efficiency for each filter layer, and in the case of an infected wearer that is shedding virus, the inside source collection filter 908 will collect a minimum of approximately 4.5 times more target particles than the receptor collection filter 910. This difference is sufficient to allow for a determination as to whether target particles originated from an infected wearer or a contaminated environment. Further, as the efficiency of each layer is increased the ratio of target particles collected by each filter increases. As an example, two 90% efficiency filters will provide a minimum of approximately 10 times more particles on source collection filter 908 if the wearer is contaminated or 10 times more on receptor collection filter 910 if the wearer is in a contaminated environment. Finally, these estimated ratios are most likely lower than they will be in actual use cases because not all particles containing target material are 0.3 μm in diameter, but are rather distributed over a large particle size range, and, in general, particles larger than and smaller than 0.3 μm in diameter are captured with higher efficiency which will lead to larger ratios.

When wearing the mask 900, exhaled and inhaled air can pass through the protective filter material 901 and through the opening 903 and collection filter assembly 913. In this exemplary embodiment, the resistance (e.g., pressure drop) for air flowing through the collection filter assembly 913 will generally be less than or equal to the resistance (e.g., pressure drop) for air flowing through an equivalent surface area of the protective filter material 901. This configuration allows for increased airflow through the collection filter assembly 913 and therefor increased collection of target particles onto the collection filter assembly 913 while providing easy access to the collection filter assembly 913 by health care workers that are processing samples. The collection filter assembly 913 may be made up of all or a portion of the layers of material used in the protective filter material 901. In this embodiment specifically, the source collection filter 908 and receptor collection filter 910 may be constructed from the internal filter material within the protective filter material 901 and the interior protective layer 906 and exterior protective layer 912 may be constructed from the cover web that is the outer layer often found within the protective filter material 901 of many N95 masks.

The source collection filter 908 and receptor collection filter 910 can be selected from a range of filter materials that will be known to those skilled in the art, including but not limited to: non-woven, woven, membrane, fiber, depth, electret, hydrophobic, or hydrophilic filtering materials. The interior protective layer 906 and exterior protective layer 912 can be selected from a range of low resistance (e.g., low pressure drop) materials that offer protection from physical contact and provide structural integrity to the collection filter assembly 913, while limiting the potential for damage to the source collection filter 908 and receptor collection filter 910. These materials, that will be known to those skilled in the art, including but not limited to: non-woven, woven, membrane, fiber, depth, hydrophobic, or hydrophilic materials.

The interior protective layer 906, source collection filter 908, receptor collection filter 910, and the exterior protective layer 912 are bonded to each other to form the collection filter assembly 913. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 913 will be removed from the mask 900 for placement into a sample tube for particle extraction, or the bond between the layers may be removable, or they may be bonded with a tear-away portion, to allow for separation of the layers. In this way, the interior protective layer 906 and exterior protective layer 912 may be discarded and the source collection filter 908 and receptor collection filter 910 may be placed into a single sample tube or separate sample tubes for shipment or for extraction.

Similarly, the collection filter assembly 913 is then bonded to the border 904 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 913 and the border 904 must be airtight and removable. In this way, when worn, a large amount of exhaled and inhaled air will pass through the collection filter assembly 913 layers and when the sampling period is complete the entire collection filter assembly may be easily removed from the mask 900.

Tab 903 of collection filter assembly 913 allows for the health care worker, wearer of the mask, or another person to easily remove the collection filter assembly 913 by grasping the interior proactive layer tab 905, source collection filter tab 907, receptor collection filter tab 909, or exterior protective layer tab 911, or a combination of the tabs together. The collection filter assembly 913 may be designed such that the interior protective layer tab 905 is the largest of the tabs and can be easily grabbed by itself or the three tabs may be bonded partially together to form a single tab that is grasped for removal. Finally, interior protective layer 906, the source collection filter 908, receptor collection filter 910, and exterior protective layer 912 can have a single combined tab that is formed by bonding a portion of the tab area together, but with a perforation that allows the top of the tab to be torn off revealing separate tabs on each layer that can be used for separation of the layers.

The described approach allows collection filter assembly 913 to be removed from mask 900 without having to remove mask 900. The person removing the collection filter assembly 913 will generally be expected to have gloved hands and will place one hand onto the outside of mask 900 near the nose area and grasp the tabs within the collection filter assembly 913 with the other hand and pull down to remove the collection filter assembly 913. Other means of removing the collection filter assembly 913, such as pulling up on the tabs within the collection filter assembly 913, with the tabs at the bottom of the collection filter assembly 913, will be apparent to those known in the art. Because collection filter assembly 913 can be easily removed from outside the mask, the potential for contamination of the health care worker or cross contamination of other samples is minimized. After removal of the collection filter assembly 913, opening 903 can be covered by placing a new collection filter assembly 913 over the opening or by placing a temporary pressure sensitive adhesive seal over the opening. In this way, it is possible for a health care worker to collect samples from a large number of people in a short period of time with minimal potential for exposure to viral or microbial aerosols. Alternatively, for home or non-health care facility use it is possible for the wearer to remove mask 900 and then remove collection filter assembly 913 from mask 900.

After removal of collection filter assembly 913, target particles captured in the entire collection filter assembly 913 may be extracted by placing the collection filter assembly 913 into a sample tube or other container and vortexing, shaking, sonicating, rocking, or with wet foam elution. Alternatively, dependent on the design of the collection filter assembly 913, it may be separated using tabs on the components and gloved hands, forceps or other tools and collection filters within the assembly may be placed into a sample container and shipped or transported to a lab for extraction or extraction may be performed in the field.

It should be noted that the material in the collection filter assembly 913 has a particle removal efficiency similar to that of protective filter material 901 and a resistance (e.g., pressure drop) generally less than or equal to the resistance (e.g., pressure drop) of an equivalent surface area of protective filter material 901. In this way, the volume of air passing through the collection filter assembly 913 is similar to that passing through an equivalent surface area of protective filter material 901 and the amount of target captured per surface area is maximized.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

FIG. 10 shows a protective mask 1000 with an alternative configuration of a collection filter assembly 1004 with a support frame 1005 and opening 1006, according to an exemplary embodiment of the present subject disclosure. The mask 1000 has and protective filter material 1001 and includes a strap 1002 for holding the mask 1000 to the wearer's face by looping over the ears or around the back of neck and head. The mask 1000 has an opening, with a perimeter, through the protective filter material 1001. The opening, which is similar to opening 103 and opening 303 shown in FIG. 1 and FIG. 3, allows a collection filter assembly 1004 to be attached to the outside of the mask 1000 with a bond sealing the collection filter assembly around the perimeter of the opening.

Thus, when wearing the mask 1000, exhaled and inhaled air can pass through the protective filter material 1001 and through the opening in the protective filter material 1001 and through the collection filter assembly 1004. This allows for increased collection of target particles into the collection filter assembly 1004 while providing easy access to the collection filter assembly by health care workers. Collection filter assembly 1004 is made up of internal collection filter and protective material layers with a support frame 1005 and a Tab 1003. The support frame can be made of a thin, flexible plastic, rigid plastic, or woven or non-woven material. Tab 1003 allows for the health care worker, wearer of the mask, or other person to easily remove the collection filter assembly 1004. Because collection filter assembly 1004 can be easily removed from outside the mask, the potential for contamination of the health care worker or cross contamination of other samples is minimized. After removal of the collection filter assembly 1004, target particles captured in the entire collection filter assembly may be extracted by placing the collection filter assembly 1004 into a sample tube or other container and vortexing, shaking, sonicating, rocking, or with wet foam elution. Alternatively, dependent on the design of the collection filter assembly 1004, the collection filter assembly 1004 may be separated using tabs on the components and glove hands, forceps or other tools and collection filters within the assembly may be placed into sample container and shipped or transported to a lab for extraction or extraction may be performed in the field.

It should be noted that the material in the collection filter assembly 1004 has a particle removal efficiency similar to that of protective filter material 1001 and a resistance (e.g., pressure drop) generally less than or equal to the resistance (e.g., pressure drop) of an equivalent surface area of protective filter material 1001. In this way, the volume of air passing through the collection filter assembly 1004 is similar to that passing through an equivalent surface area of protective filter material 1001 and the amount of target captured per surface area is maximized.

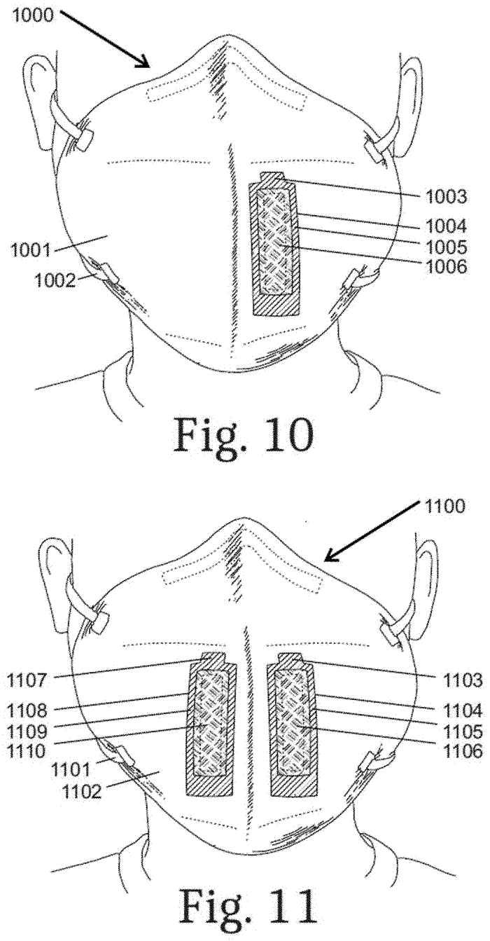

FIG. 11 shows a protective mask 1100 with an alternative configuration of two collection filter assemblies 1104 and 1108 with support frames 1015 and 1019 and openings 1106 and 1109, according to an exemplary embodiment of the present subject disclosure. This embodiment is similar to that shown in FIG. 10, but in the case of FIG. 11 protective mask 1100 contains two collection filter assemblies 1114 and 1118. The mask 1100 has a protective filter material 1101 and includes a strap 1102 for holding the mask 1100 to the wearer's face by looping over the ears or around the back of neck and head. The mask 1100 has an opening, with a perimeter, through the protective filter material 1101. The opening, which is similar to opening 103 and opening 303 shown in FIG. 1 and FIG. 3, allows a collection filter assembly 1114 and collection filter assembly 1118 to be attached to the outside of the mask 1100 with a bond sealing the collection filter assemblies around the perimeter of the opening. Thus, when wearing the mask, exhaled and inhaled air can pass through the protective filter material 1101 and through the openings in the protective filter material and through the collection filter assembly 1114 and collection filter assembly 1118. This allows for increased collection of target particles into the collection filter assembly while providing easy access to the collection filter assembly by health care workers. Collection filter assembly 1114 and collection filter assembly 1118 are each made up of internal collection filter and protective material layers with support frame 1115 and support frame 1119, respectively, and tab 1113 and tab 1117, respectively. The support frame can be made of a thin, flexible plastic, rigid plastic, or woven or non-woven material.

Tab 1113 on collection filter assembly 1114 allows for the health care worker, wearer of the mask, or other person to easily remove collection filter assembly 1114. Because collection filter assembly 1114 can be easily removed from outside the mask, the potential for contamination of the health care worker or cross contamination of other samples is minimized. Similarly, collection filter assembly 1118 can be easily removed by grasping and peeling away tab 1117. After removal of the collection filter assembly 1114 and collection filter assembly 1118, target particles captured in the entire collection filter assembly structure may be extracted by placing the collection filter assembly 1114 and collection filter assembly 1118 into a sample tube or other container and vortexing, shaking, sonicating, rocking, or with wet foam elution. Alternatively, dependent on the design of the collection filter assembly 1114 and collection filter assembly 1118, the collection filter assemblies may be separated using tabs on the components and glove hands, forceps or other tools and collection filters within the assembly may be placed into sample container and shipped or transported to a lab for extraction or extraction may be performed in the field.

It should be noted that the material in collection filter assembly 1114 and collection filter assembly 1118 have particle removal efficiencies similar to that of protective filter material 1111 and a resistance (e.g., pressure drop) generally less than or equal to the resistance (e.g., pressure drop) of an equivalent surface area of protective filter material 1111. In this way, the volume of air passing through collection filter assembly 1114 and collection filter assembly 1118 is similar to that passing through an equivalent surface area of protective filter material 1111 and the amount of target captured per surface area is maximized.

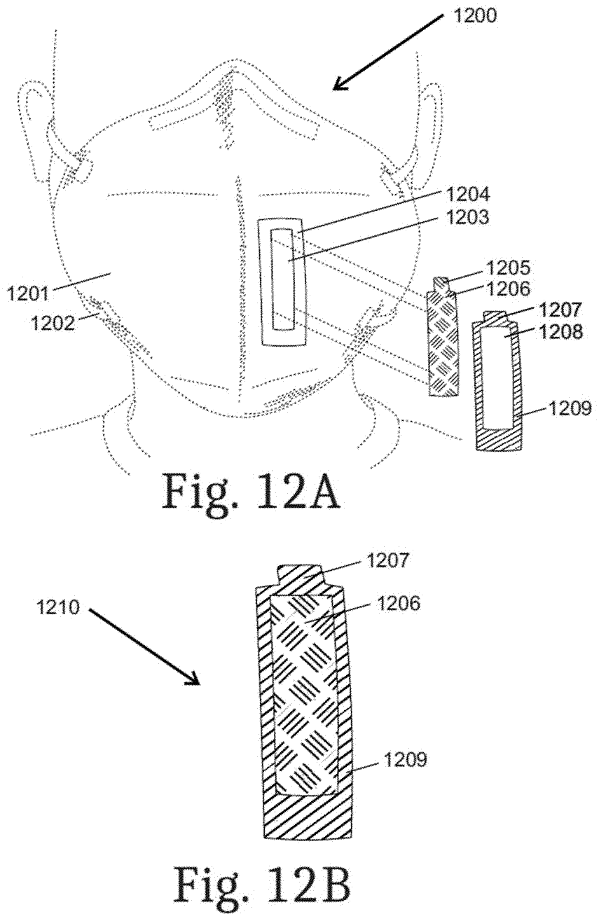

FIGS. 12A and 12B show a protective mask 1200 with a two-layer collection filter assembly 1210 with support frame 1209, according to an exemplary embodiment of the present subject disclosure. The protective mask 1200 has a protective filter material 1201 and includes a strap 1202 for holding the mask 1200 to the wearer's face by looping over the ears or around the back of neck and head. The mask 1200 has an opening 1203, with a perimeter 1204, through the protective filter material 1201. The opening 1203 allows a collection filter assembly 1210 to be attached to the outside of mask 1200 with a bond sealing the collection filter assembly around the perimeter of the opening.

Collection filter assembly 1210 is made up of collection filter 1206 with collection filter tab 1205 and support frame 1209 with opening 1208 and support frame tab 1207. When wearing the mask 1200, exhaled and inhaled air can pass through the protective filter material 1201 and through the opening 1203 and collection filter assembly 1210. In this exemplary embodiment, the resistance (e.g., pressure drop) for air flowing through the collection filter assembly 1210 will generally be less than equal to the resistance (e.g., pressure drop) for air flowing through an equivalent surface area of the protective filter material 1201. This configuration allows for increased airflow through the collection filter assembly 1210 and therefor increased collection of target particles onto the collection filter assembly 1210 while providing easy access to the collection filter assembly 1210 by health care workers. The collection filter assembly 1210 may be made up of all or a portion of the layers of material used in the protective filter material 1201. In this embodiment specifically, the collection filter 1206 may be constructed from the internal filter material that is primary filter layer within the protective filter material 1201 of many N95 masks.

The collection filter 1206 can be selected from a range of filter materials that will be known to those skilled in the art, including but not limited to: non-woven, woven, membrane, fiber, depth, electret, hydrophobic, or hydrophilic filtering materials. The frame 1209 can be selected from a range of materials including thin, flexible plastic sheet material, hard plastics, rubbers, woven fabric, non-woven fabric, and membrane materials. The frame 1209 offers structural integrity to the collection filter assembly 1210, while limiting the potential for damage to the collection filter 1206 during removal from the mask.

The collection filter 1206 and the frame 1209 are bonded to each other to form the collection filter assembly 1210. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1210 will be removed from the mask 1200 for placement into a sample tube for particle extraction, or the bond between the layers may be removable, or they may be bonded with a tear-away portion, to allow for separation of the layers. In this way, the frame 1209 may be discarded and the collection filter 1206 may be placed into a sample tube for extraction.

Similarly, the collection filter assembly 1210 is then bonded to the border 1204 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1210 and the border 1204 must be airtight and removable. In this way, when worn, a large amount of exhaled and inhaled air will pass through the collection filter assembly 1210 layers and when the sampling period is complete the entire collection filter assembly may be easily removed from the mask 1200.

A tab on the collection filter assembly 1210 allows for the health care worker, wearer of the mask, or other person to easily remove the collection filter assembly 1210 from the mask 1200. The worker grasps the collection filter tab 1205, or frame tab 1207, or both together. The person removing the collection filter assembly 1210 will generally be expected to have gloved hands and will place one hand onto the top of the nose area of the mask 1200 and grasp the collection filter tab 1205 and protective layer tab 1207 with the other hand and pull down to remove the collection filter assembly 1210. Other means of removing the collection filter assembly 1210, such as pulling up on the collection filter tab 1205 and protective layer tab 1207, with the tabs at the bottom of the collection filter assembly 1210, will be apparent to those known in the art. Because collection filter assembly 1210 can be easily removed from outside the mask, the potential for contamination of the health care worker or cross contamination of other samples is minimized.

After removal of the collection filter assembly 1210, target particles captured in the entire collection filter assembly may be extracted by placing the collection filter assembly 1210 into a sample tube or other container and vortexing, shaking, sonicating, rocking, or with wet foam elution. Alternatively, dependent on the design of the collection filter assembly 1210, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1210 has a particle removal efficiency similar to that of protective filter material 1201 and a resistance (e.g., pressure drop) generally less than or equal to the resistance (e.g., pressure drop) of an equivalent surface area of protective filter material 1201. In this way, the volume of air passing through the collection filter assembly 1210 is similar to that passing through an equivalent surface area of protective filter material 1201 and the amount of target captured per surface area is maximized. Further, this ensures that the wearer of the mask 1200 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

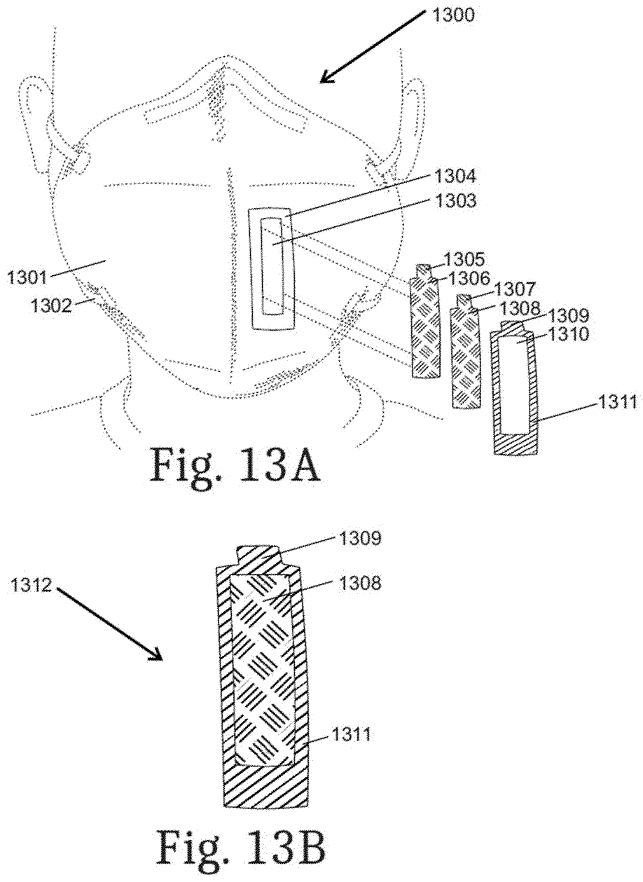

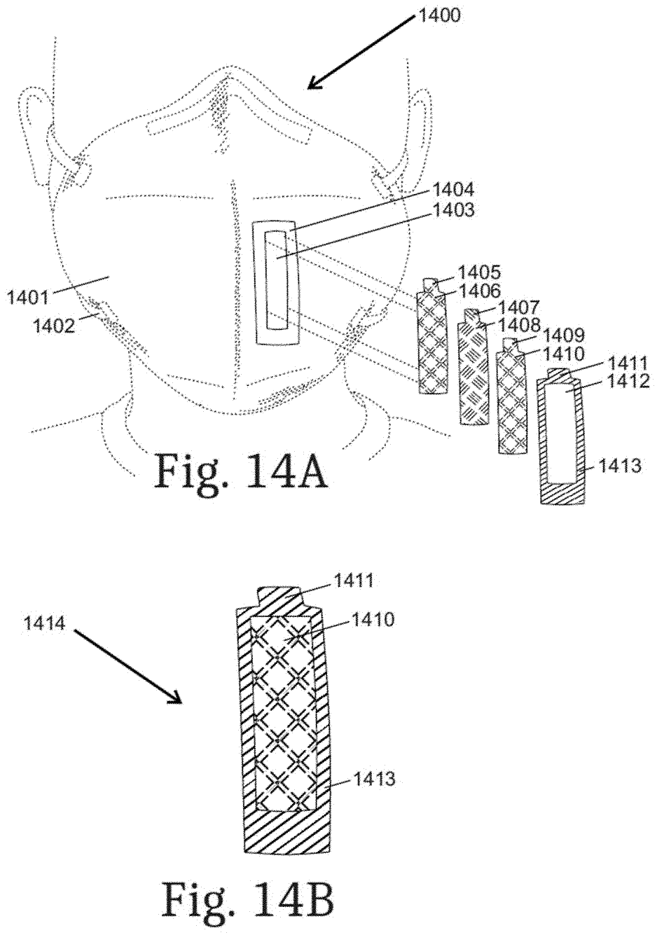

FIGS. 13A and 13B show a protective mask 1300 with a three-layer collection filter assembly with support frame 1311, according to an exemplary embodiment of the present subject disclosure. The protective mask 1300 has a protective filter material 1301 and includes a strap 1302 for holding the mask 1300 to the wearer's face by looping over the ears or around the back of neck and head. The mask 1300 has an opening 1303, with a perimeter 1304, through the protective filter material 1301. The opening 1303 allows a collection filter assembly 1312 to be attached to the outside of mask 1300 with a bond sealing the collection filter assembly around the perimeter of the opening.

Collection filter assembly 1312 is made up of source collection filter 1306 with source collection filter tab 1305, receptor collection filter 1308 with receptor collection filter tab 1307, and support frame 1311 with opening 1310 and support frame tab 1309. When wearing the mask 1300, exhaled and inhaled air can pass through the protective filter material 1301 and through the opening 1303 and collection filter assembly 1312. In this exemplary embodiment, the resistance (e.g., pressure drop) for air flowing through the collection filter assembly 1312 will generally be less than equal to the resistance (e.g., pressure drop) for air flowing through an equivalent surface area of the protective filter material 1301. This configuration allows for increased airflow through the collection filter assembly 1312 and therefor increased collection of target particles onto the collection filter assembly 1312 while providing easy access to the collection filter assembly 1312 by health care workers. The collection filter assembly 1312 may be made up of all or a portion of the layers of material used in the protective filter material 1301. In this embodiment specifically, the source collection filter 1306 and receptor collection filter 1308 may be constructed from the internal filter material that is primary filter layer within the protective filter material 1301 of many N95 masks.

The source collection filter 1306 and receptor collection filter 1308 can be selected from a range of filter materials that will be known to those skilled in the art, including but not limited to: non-woven, woven, membrane, fiber, depth, electret, hydrophobic, or hydrophilic filtering materials. Further, source collection filter 1306 and receptor collection filter 1307 may both be constructed from the same material or they may be constructed from different materials with different properties. The frame 1311 can be selected from a range of materials including thin, flexible plastic sheet material, hard plastics, rubbers, woven fabric, non-woven fabric, and membrane materials. The frame 1311 offers structural integrity to the collection filter assembly 1312, while limiting the potential for damage to the source collection filter 1306 and receptor collection filter 1307 during use and during removal from the mask.

The source collection filter 1306, receptor collection filter 1307, and the frame 1309 are bonded together to form the collection filter assembly 1312. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1312 will be removed from the mask 1300 for placement into a sample tube for particle extraction, or the bond between the layers may be removable, or they may be bonded with a tear-away portion, to allow for separation of the layers. In this way, the frame 1309 may be discarded and source collection filter 1306 and receptor collection filter 1307 may be placed into a single sample tube or two separate sample tubes for transport to a laboratory or for immediate extraction.

The source collection filter 1306 and receptor collection filter 1307 may be transported dry or wet. For dry transport a desiccant pack may be included in the transport kit to assist the filters in drying and as such helping maintain certain target materials. Alternatively, a liquid buffer may be supplied for the user to add to the sample tube along with the sample. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Similar to the above, the collection filter assembly 1312 is bonded to the border 1304 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1312 and the border 1304 must be airtight and removable. In this way, when worn, a large amount of exhaled and inhaled air will pass through the collection filter assembly 1312 layers and when the sampling period is complete the entire collection filter assembly may be easily removed from the mask 1300.

A tab on the collection filter assembly 1312 allow for the health care worker, wearers of the mask, or other persons to easily remove the collection filter assembly 1312 from the mask 1300. The worker grasps the source collection filter tab 1305, receptor collection filter tab 1307, or frame tab 1309, or a combination of the tabs. The person removing the collection filter assembly 1312 will generally be expected to have gloved hands and will place one hand onto the top of the nose area of the mask 1300 and grasp a combination or one of the tabs with the other hand and pull down to remove the collection filter assembly 1312. Other means of removing the collection filter assembly 1312, such as pulling up on the tabs, which in this case are located at the bottom of the collection filter assembly 1312, will be apparent to those known in the art. Because collection filter assembly 1312 can be easily removed from outside the mask, the potential for contamination of the health care worker or cross contamination of other samples is minimized.

After removal of the collection filter assembly 1312, target particles captured in the entire collection filter assembly may be extracted by placing the collection filter assembly 1312 into a sample tube or other container and vortexing, shaking, sonicating, rocking, or with wet foam elution. Alternatively, dependent on the design of the collection filter assembly 1312, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1312 has a particle removal efficiency similar to that of protective filter material 1301 and a resistance (e.g., pressure drop) generally less than or equal to the resistance (e.g., pressure drop) of an equivalent surface area of protective filter material 1301. In this way, the volume of air passing through the collection filter assembly 1312 is similar to that passing through an equivalent surface area of protective filter material 1301 and the amount of target captured per surface area is maximized. Further, this ensures that the wearer of the mask 1300 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

FIGS. 14A and 14B show a protective mask 1400 with a four-layer collection filter assembly 1414 with support frame, according to an exemplary embodiment of the present subject disclosure. The protective mask 1400 has a protective filter material 1401 and includes a strap 1402 for holding the mask 1400 to the wearer's face by looping over the ears or around the back of neck and head. The mask 1400 has an opening 1403, with a perimeter 1404, through the protective filter material 1401. The opening 1403 allows a collection filter assembly 1414 to be attached to the outside of mask 1400 with a bond sealing the collection filter assembly around the perimeter of the opening.

Collection filter assembly 1414 is made up of interior protective layer 1406 with interior protective layer tab 1405, collection filter 1408 with collection filter tab 1407, exterior protective layer 1410 with exterior protective layer tab 1409, and support frame 1413 with opening 1412 and support frame tab 1411. When wearing the mask 1400, exhaled and inhaled air can pass through the protective filter material 1401 and through the opening 1403 and collection filter assembly 1414. In this exemplary embodiment, the resistance (e.g., pressure drop) for air flowing through the collection filter assembly 1414 will generally be less than equal to the resistance (e.g., pressure drop) for air flowing through an equivalent surface area of the protective filter material 1401. This configuration allows for increased airflow through the collection filter assembly 1414 and therefor increased collection of target particles onto the collection filter assembly 1414 while providing easy access to the collection filter assembly 1414 by health care workers. The collection filter assembly 1414 may be made up of all or a portion of the layers of material used in the protective filter material 1401. In this embodiment specifically, the collection filter 1408 may be constructed from the internal filter material that is primary filter layer within the protective filter material 1401 of many N95 masks.

The collection filter 1408 can be selected from a range of filter materials that will be known to those skilled in the art, including but not limited to: non-woven, woven, membrane, fiber, depth, electret, hydrophobic, or hydrophilic filtering materials. The frame 1413 can be selected from a range of materials including thin, flexible plastic sheet material, hard plastics, rubbers, woven fabric, non-woven fabric, and membrane materials. The frame 1413 offers structural integrity to the collection filter assembly 1414, while limiting the potential for damage or contamination of collection filter 1408 during use and during removal from the mask.

The interior protective layer 1406 with interior protective layer tab 1405, collection filter 1408 with collection filter tab 1407, exterior protective layer 1410 with exterior protective layer tab 1409, and support frame 1413 with opening 1412 and support frame tab 1411 are bonded together to form the collection filter assembly 1414. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1414 will be removed from the mask 1400 for placement into a sample tube for particle extraction, or the bond between the layers may be removable, or they may be bonded with a tear-away portion, to allow for separation of the layers. In this way, the collection filter 1408 may be placed into a sample tube for transport to a laboratory or for immediate extraction.

The collection filter 1408 may be transported dry or wet. For dry transport a desiccant pack may be included in the transport kit to assist the filters in drying and as such helping maintain certain target materials. Alternatively, a liquid buffer may be supplied for the user to add to the sample tube along with the sample. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Similar to the above, the collection filter assembly 1414 is bonded to the border 1404 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1414 and the border 1404 must be airtight and removable. In this way, when worn, a large amount of exhaled and inhaled air will pass through the collection filter assembly 1414 layers and when the sampling period is complete the entire collection filter assembly may be easily removed from the mask 1400.

A tab on the collection filter assembly 1414 allow for the health care worker, wearers of the mask, or other persons to easily remove the collection filter assembly 1414 from the mask 1400. The person removing the collection filter assembly 1414 will generally be expected to have gloved hands and will place one hand onto the top of the nose area of the mask 1400 and grasp a combination or one of the tabs with the other hand and pull down to remove the collection filter assembly 1414. Other means of removing the collection filter assembly 1414, such as pulling up on the tabs, which in this case are located at the bottom of the collection filter assembly 1414, will be apparent to those known in the art. Because collection filter assembly 1414 can be easily removed from outside the mask, the potential for contamination of the health care worker or cross contamination of other samples is minimized.

After removal of the collection filter assembly 1414, target particles captured in the entire collection filter assembly may be extracted by placing the collection filter assembly 1414 into a sample tube or other container and vortexing, shaking, sonicating, rocking, or with wet foam elution. Alternatively, dependent on the design of the collection filter assembly 1414, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1414 has a particle removal efficiency similar to that of protective filter material 1401 and a resistance (e.g., pressure drop) generally less than or equal to the resistance (e.g., pressure drop) of an equivalent surface area of protective filter material 1401. In this way, the volume of air passing through the collection filter assembly 1414 is similar to that passing through an equivalent surface area of protective filter material 1401 and the amount of target captured per surface area is maximized. Further, this ensures that the wearer of the mask 1400 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

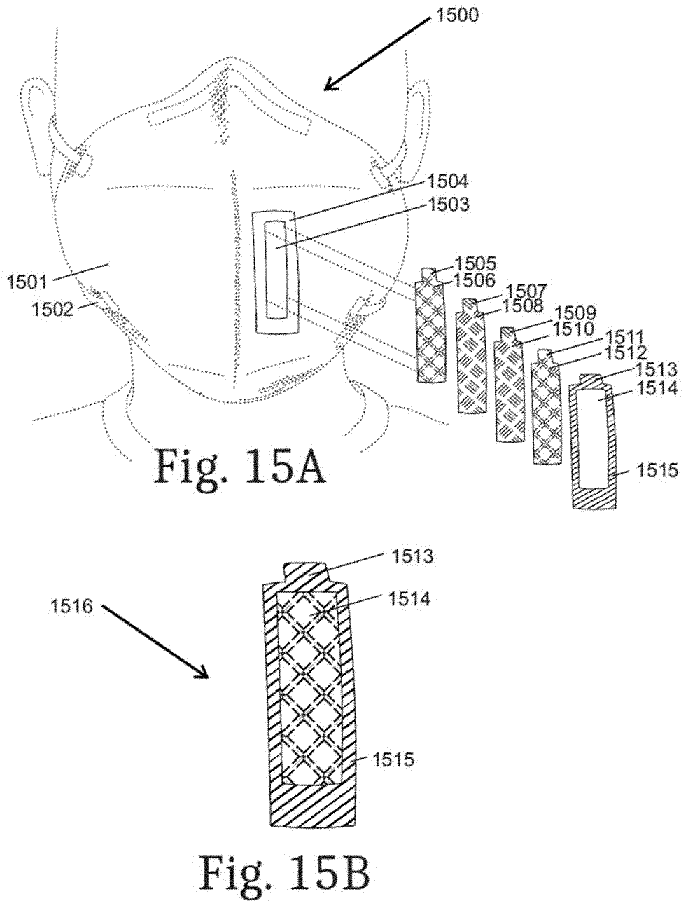

FIGS. 15A and 15B show a protective mask with a five-layer collection filter assembly 1516 with support frame 1515, according to an exemplary embodiment of the present subject disclosure. The protective mask 1500 has a protective filter material 1501 and includes a strap 1502 for holding the mask 1500 to the wearer's face by looping over the ears or around the back of neck and head. The mask 1500 has an opening 1503, with a perimeter 1504, through the protective filter material 1501. The opening 1503 allows a collection filter assembly 1516 to be attached to the outside of mask 1500 with a bond sealing the collection filter assembly around the perimeter of the opening.

Collection filter assembly 1516 is made up of interior protective layer 1506 with interior protective layer tab 1505, source collection filter 1508 with source collection filter tab 1507, receptor collection filter 1510 with receptor collection filter tab 1509, exterior protective layer 1512 with exterior protective layer tab 1511, and support frame 1515 with opening 1514 and support frame tab 1513. When wearing the mask 1500, exhaled and inhaled air can pass through the protective filter material 1501 and through the opening 1503 and collection filter assembly 1516. In this exemplary embodiment, the resistance (e.g., pressure drop) for air flowing through the collection filter assembly 1516 will generally be less than equal to the resistance (e.g., pressure drop) for air flowing through an equivalent surface area of the protective filter material 1501. This configuration allows for increased airflow through the collection filter assembly 1516 and therefor increased collection of target particles onto the collection filter assembly 1516 while providing easy access to the collection filter assembly 1516 by health care workers. The collection filter assembly 1516 may be made up of all or a portion of the layers of material used in the protective filter material 1501. In this embodiment specifically, the source collection filter 1508 and receptor collection filter 1510 may be constructed from the internal filter material that is primary filter layer within the protective filter material of many N95 masks. Further interior protective layer 1506 and exterior protective layer 1512 may be constructed from the cover web material that is found within the protective filter material of many N95 masks.

The source collection filter 1508 and receptor collection filter 1510 can be selected from a range of filter materials that will be known to those skilled in the art, including but not limited to: non-woven, woven, membrane, fiber, depth, electret, hydrophobic, or hydrophilic filtering materials. Further, source collection filter 1508 and receptor collection filter 1510 may both be constructed from the same material or they may be constructed from different materials with different properties. The frame 1515 can be selected from a range of materials including thin, flexible plastic sheet material, hard plastics, rubbers, woven fabric, non-woven fabric, and membrane materials. The frame 1515 offers structural integrity to the collection filter assembly 1516, while limiting the potential for damage to the source collection filter 1508 and receptor collection filter 1510 during use and during removal from the mask.

Interior protective layer 1506 with interior protective layer tab 1505, source collection filter 1508 with source collection filter tab 1507, receptor collection filter 1510 with receptor collection filter tab 1509, exterior protective layer 1512 with exterior protective layer tab 1511, and support frame 1515 with opening 1514 and support frame tab 1513 are bonded together to form the collection filter assembly 1516. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1516 will be removed from the mask 1500 for placement into a sample tube for particle extraction, or the bond between the layers may be removable, or they may be bonded with a tear-away portion, to allow for separation of the layers. In this way, the frame 1515, interior protective layer tab 1505, and exterior protective layer 1512 may be discarded and source collection filter 1508 and receptor collection filter 1510 may be placed into a single sample tube or two separate sample tubes for transport to a laboratory or for immediate extraction.

The source collection filter 1508 and receptor collection filter 1510 may be transported dry or wet. For dry transport a desiccant pack may be included in the transport kit to assist the filters in drying and as such helping maintain certain target materials. Alternatively, a liquid buffer may be supplied for the user to add to the sample tube along with the sample. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Similar to the above, the collection filter assembly 1516 is bonded to the border 1504 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1516 and the border 1504 must be airtight and removable. In this way, when worn, a large amount of exhaled and inhaled air will pass through the collection filter assembly 1516 layers and when the sampling period is complete the entire collection filter assembly may be easily removed from the mask 1500.

A tab on the collection filter assembly 1516 allow for the health care worker, wearers of the mask, or other persons to easily remove the collection filter assembly 1516 from the mask 1500. The tabs may be bonded to each other or separated in a variety of ways to facilitate removal of the collection filter assembly 1516 from the mask 1500 and to facilitate separation into layers. The person removing the collection filter assembly 1516 will generally be expected to have gloved hands and will place one hand onto the top of the nose area of the mask 1500 and grasp a combination or one of the tabs with the other hand and pull down to remove the collection filter assembly 1516. Other means of removing the collection filter assembly 1516, such as pulling up on the tabs, which in this case are located at the bottom of the collection filter assembly 1516, will be apparent to those known in the art. Because collection filter assembly 1516 can be easily removed from outside the mask, the potential for contamination of the health care worker or cross contamination of other samples is minimized.

After removal of the collection filter assembly 1516, target particles captured in the entire collection filter assembly may be extracted by placing the collection filter assembly 1516 into a sample tube or other container and vortexing, shaking, sonicating, rocking, or with wet foam elution. Alternatively, dependent on the design of the collection filter assembly 1516, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1516 has a particle removal efficiency similar to that of protective filter material 1501 and a resistance (e.g., pressure drop) generally less than or equal to the resistance (e.g., pressure drop) of an equivalent surface area of protective filter material 1501. In this way, the volume of air passing through the collection filter assembly 1516 is similar to that passing through an equivalent surface area of protective filter material 1501 and the amount of target captured per surface area is maximized. Further, this ensures that the wearer of the mask 1500 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

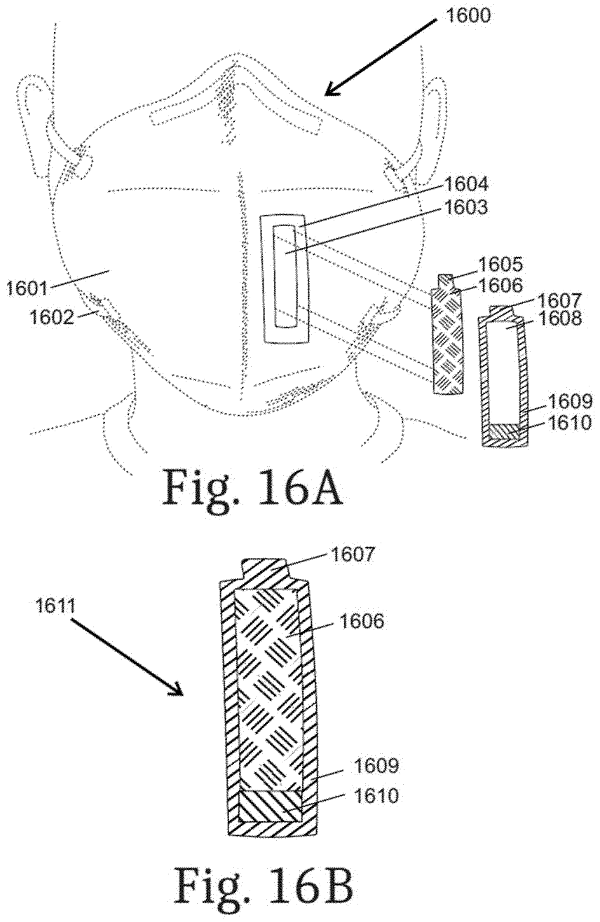

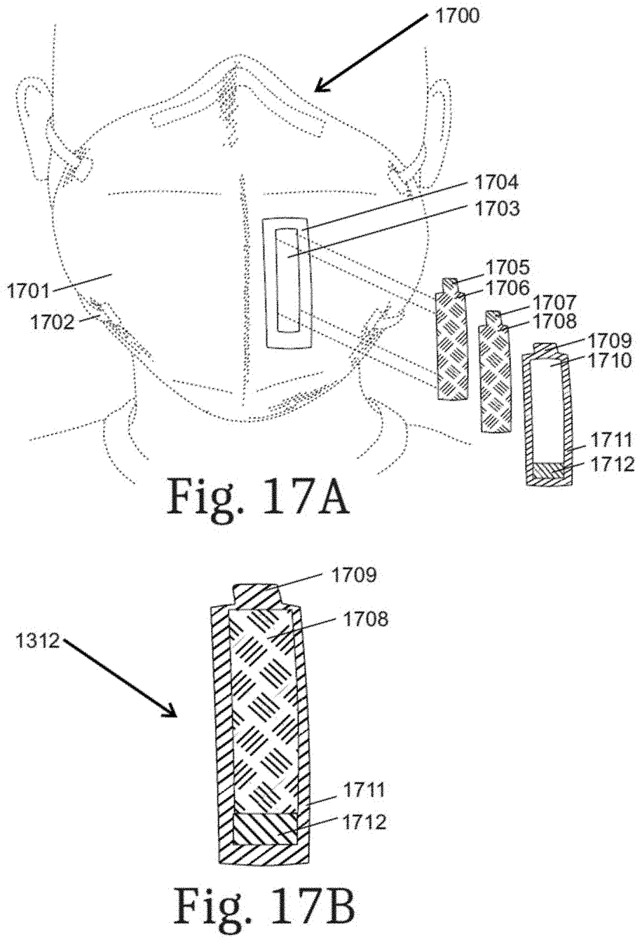

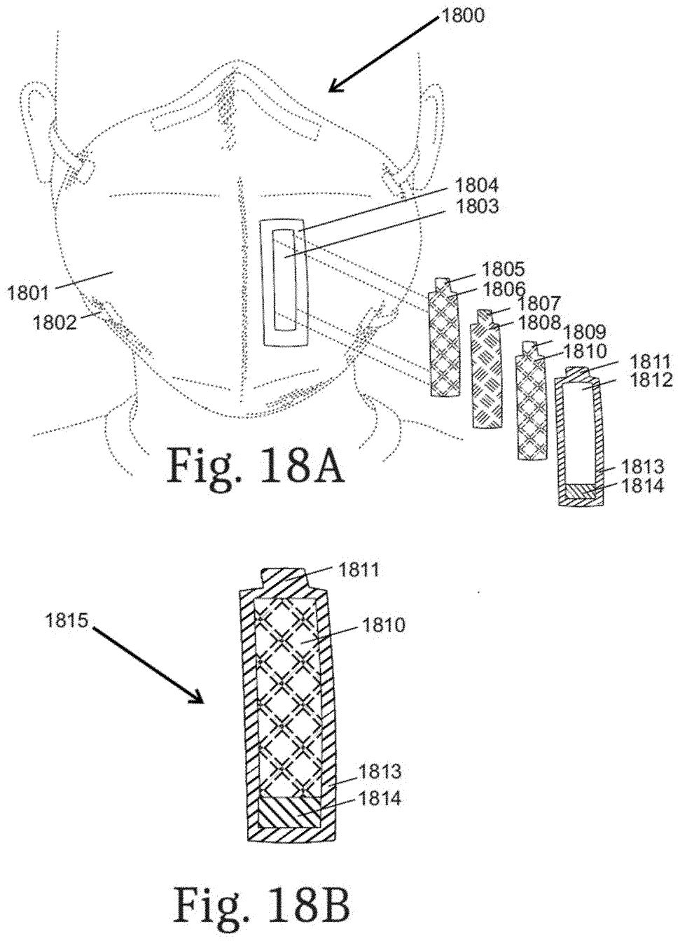

FIGS. 16A and 16B show a protective mask 1600 with a two-layer collection filter assembly 1611 with a usage indicator 1610 and support frame 1609 according to an exemplary embodiment of the present subject disclosure. The protective mask 1600 has a protective filter material 1601 and includes a strap 1602 for holding the mask 1600 to the wearer's face by looping over the ears or around the back of neck and head. The mask 1600 has an opening 1603, with a perimeter 1604, through the protective filter material 1601. The opening 1603 allows a collection filter assembly 1611 to be attached to the outside of mask 1600 with a bond sealing the collection filter assembly around the perimeter of the opening.

Collection filter assembly 1611 is made up of collection filter 1606 with collection filter tab 1605, usage indicator 1610, and support frame 1609 with opening 1608 and support frame tab 1607. When wearing the mask 1600, exhaled and inhaled air can pass through the protective filter material 1601 and through the opening 1603 and collection filter assembly 1611, as well as by or through usage indicator 1610. In this exemplary embodiment, the resistance (e.g., pressure drop) for air flowing through the collection filter assembly 1611 will generally be less than equal to the resistance (e.g., pressure drop) for air flowing through an equivalent surface area of the protective filter material 1601. This configuration allows for increased airflow through the collection filter assembly 1611 and therefor increased collection of target particles onto the collection filter assembly 1611 while providing easy access to the collection filter assembly 1611 by health care workers. The collection filter assembly 1611 may be made up of all or a portion of the layers of material used in the protective filter material 1601. In this embodiment specifically, the collection filter 1606 may be constructed from the internal filter material that is primary filter layer within the protective filter material 1601 of many N95 masks.

The collection filter 1606 can be selected from a range of filter materials that will be known to those skilled in the art, including but not limited to: non-woven, woven, membrane, fiber, depth, electret, hydrophobic, or hydrophilic filtering materials. The frame 1609 can be selected from a range of materials including thin, flexible plastic sheet material, hard plastics, rubbers, woven fabric, non-woven fabric, and membrane materials. The frame 1609 offers structural integrity to the collection filter assembly 1611, while limiting the potential for damage to the collection filter 1606 during removal from the mask.

The usage indicator 1610 utilizes a pH indicator or moisture indicator to provide a visible signal to the user, health care worker, or laboratory worker that the mask has been worn for a sufficient time period. Exhaled carbon dioxide is readily soluble in water—forming carbonic acid. When in a high humidity environment carbonic acid is formed in water captured onto surfaces and within absorbent materials. To produce usage indicator 1610, standard pH indicators, like those used in pH indicator strips, are impregnated onto a solid substrate or into a porous substrate. Said substrate is integrated into the collection filter assembly 1611 as part of a filter, protective layer, or support frame 1609. The usage indicator 1610 can be designed to be visible through opening 1608 with or without a clear covering over the indicator. The usage indicator 1610 may also be on the inside surface of the collection filter assembly 1611, such that it is only visible after removal from mask 1600 or by removing the mask from the wearer and looking through opening 1603. Further, the indicator may also be contained within the collection filter assembly 1611, such that it is only visible after removal from the mask 1600 and separation of the layers.

The collection filter 1606 and the frame 1609 are bonded to each other to form the collection filter assembly 1611. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1611 will be removed from the mask 1600 for placement into a sample tube for particle extraction, or the bond between the layers may be removable, or they may be bonded with a tear-away portion, to allow for separation of the layers. In this way, the frame 1609 may be discarded and the collection filter 1606 may be placed into a sample tube for extraction.

Similarly, the collection filter assembly 1611 is then bonded to the border 1604 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1611 and the border 1604 must be airtight and removable. In this way, when worn, a large amount of exhaled and inhaled air will pass through the collection filter assembly 1611 layers and when the sampling period is complete the entire collection filter assembly may be easily removed from the mask 1600.

A tab on the collection filter assembly 1611 allows for the health care worker, wearer of the mask, or other person to easily remove the collection filter assembly 1611 from the mask 1600. The worker grasps the collection filter tab 1605, or frame tab 1607, or both together. The person removing the collection filter assembly 1611 will generally be expected to have gloved hands and will place one hand onto the top of the nose area of the mask 1600 and grasp the collection filter tab 1605 and protective layer tab 1607 with the other hand and pull down to remove the collection filter assembly 1611. Other means of removing the collection filter assembly 1611, such as pulling up on the collection filter tab 1605 and protective layer tab 1607, with the tabs at the bottom of the collection filter assembly 1611, will be apparent to those known in the art. Because collection filter assembly 1611 can be easily removed from outside the mask, the potential for contamination of the health care worker or cross contamination of other samples is minimized.

After removal of the collection filter assembly 1611, target particles captured in the entire collection filter assembly may be extracted by placing the collection filter assembly 1611 into a sample tube or other container and vortexing, shaking, sonicating, rocking, or with wet foam elution. Alternatively, dependent on the design of the collection filter assembly 1611, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1611 has a particle removal efficiency similar to that of protective filter material 1601 and a resistance (e.g., pressure drop) generally less than or equal to the resistance (e.g., pressure drop) of an equivalent surface area of protective filter material 1601. In this way, the volume of air passing through the collection filter assembly 1611 is similar to that passing through an equivalent surface area of protective filter material 1601 and the amount of target captured per surface area is maximized. Further, this ensures that the wearer of the mask 1600 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

FIGS. 17A and 17B show a protective mask 1700 with a three-layer collection filter assembly 1713 with a usage indicator 1712 and support frame 1711, according to an exemplary embodiment of the present subject disclosure. The protective mask 1700 has a protective filter material 1701 and includes a strap 1702 for holding the mask 1700 to the wearer's face by looping over the ears or around the back of neck and head. The mask 1700 has an opening 1703, with a perimeter 1704, through the protective filter material 1701. The opening 1703 allows a collection filter assembly 1713 to be attached to the outside of mask 1700 with a bond sealing the collection filter assembly around the perimeter of the opening.

Collection filter assembly 1713 is made up of source collection filter 1706 with source collection filter tab 1705, receptor collection filter 1708 with receptor collection filter tab 1707, usage indicator 1712, and support frame 1711 with opening 1710 and support frame tab 1709. When wearing the mask 1700, exhaled and inhaled air can pass through the protective filter material 1701 and through the opening 1703 and collection filter assembly 1713, as well as by or through usage indicator 1712. In this exemplary embodiment, the resistance (e.g., pressure drop) for air flowing through the collection filter assembly 1713 will generally be less than equal to the resistance (e.g., pressure drop) for air flowing through an equivalent surface area of the protective filter material 1701. This configuration allows for increased airflow through the collection filter assembly 1713 and therefor increased collection of target particles onto the collection filter assembly 1713 while providing easy access to the collection filter assembly 1713 by health care workers. The collection filter assembly 1713 may be made up of all or a portion of the layers of material used in the protective filter material 1701. In this embodiment specifically, the source collection filter 1706 and receptor collection filter 1708 may be constructed from the internal filter material that is primary filter layer within the protective filter material 1701 of many N95 masks.

The source collection filter 1706 and receptor collection filter 1708 can be selected from a range of filter materials that will be known to those skilled in the art, including but not limited to: non-woven, woven, membrane, fiber, depth, electret, hydrophobic, or hydrophilic filtering materials. Further, source collection filter 1706 and receptor collection filter 1707 may both be constructed from the same material or they may be constructed from different materials with different properties. The frame 1711 can be selected from a range of materials including thin, flexible plastic sheet material, hard plastics, rubbers, woven fabric, non-woven fabric, and membrane materials. The frame 1711 offers structural integrity to the collection filter assembly 1713, while limiting the potential for damage to the source collection filter 1706 and receptor collection filter 1707 during use and during removal from the mask.

The usage indicator 1712 utilizes a pH indicator or moisture indicator to provide a visible signal to the user, health care worker, or laboratory worker that the mask has been worn for a sufficient time period. Exhaled carbon dioxide is readily soluble in water—forming carbonic acid. When in a high humidity environment carbonic acid is formed in water captured onto surfaces and within absorbent materials. To produce usage indicator 1712, standard pH indicators, like those used in pH indicator strips, are impregnated onto a solid substrate or into a porous substrate. Said substrate is integrated into the collection filter assembly 1713 as part of a filter, protective layer, or support frame 1711. The usage indicator 1712 can be designed to be visible through opening 1710 with or without a clear covering over the indicator. The usage indicator 1712 may also be on the inside surface of the collection filter assembly 1713, such that it is only visible after removal from mask 1700 or by removing the mask from the wearer and looking through opening 1703. Further, the indicator may also be contained within the collection filter assembly 1713, such that it is only visible after removal from the mask 1700 and separation of the layers.

The source collection filter 1706, receptor collection filter 1707, and the frame 1709 are bonded together to form the collection filter assembly 1713. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1713 will be removed from the mask 1700 for placement into a sample tube for particle extraction, or the bond between the layers may be removable, or they may be bonded with a tear-away portion, to allow for separation of the layers. In this way, the frame 1709 may be discarded and source collection filter 1706 and receptor collection filter 1707 may be placed into a single sample tube or two separate sample tubes for transport to a laboratory or for immediate extraction.

The source collection filter 1706 and receptor collection filter 1707 may be transported dry or wet. For dry transport a desiccant pack may be included in the transport kit to assist the filters in drying and as such helping maintain certain target materials. Alternatively, a liquid buffer may be supplied for the user to add to the sample tube along with the sample. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Similar to the above, the collection filter assembly 1713 is bonded to the border 1704 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1713 and the border 1704 must be airtight and removable. In this way, when worn, a large amount of exhaled and inhaled air will pass through the collection filter assembly 1713 layers and when the sampling period is complete the entire collection filter assembly may be easily removed from the mask 1700.

A tab on the collection filter assembly 1713 allow for the health care worker, wearers of the mask, or other persons to easily remove the collection filter assembly 1713 from the mask 1700. The worker grasps the source collection filter tab 1705, receptor collection filter tab 1707, or frame tab 1709, or a combination of the tabs. The person removing the collection filter assembly 1713 will generally be expected to have gloved hands and will place one hand onto the top of the nose area of the mask 1700 and grasp a combination or one of the tabs with the other hand and pull down to remove the collection filter assembly 1713. Other means of removing the collection filter assembly 1713, such as pulling up on the tabs, which in this case are located at the bottom of the collection filter assembly 1713, will be apparent to those known in the art. Because collection filter assembly 1713 can be easily removed from outside the mask, the potential for contamination of the health care worker or cross contamination of other samples is minimized.

After removal of the collection filter assembly 1713, target particles captured in the entire collection filter assembly may be extracted by placing the collection filter assembly 1713 into a sample tube or other container and vortexing, shaking, sonicating, rocking, or with wet foam elution. Alternatively, dependent on the design of the collection filter assembly 1713, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1713 has a particle removal efficiency similar to that of protective filter material 1701 and a resistance (e.g., pressure drop) generally less than or equal to the resistance (e.g., pressure drop) of an equivalent surface area of protective filter material 1701. In this way, the volume of air passing through the collection filter assembly 1713 is similar to that passing through an equivalent surface area of protective filter material 1701 and the amount of target captured per surface area is maximized. Further, this ensures that the wearer of the mask 1700 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

FIGS. 18A and 18B show a protective mask with a four-layer collection filter assembly 1815 with a usage indicator 1814 and support frame 1813, according to an exemplary embodiment of the present subject disclosure. The protective mask 1800 has a protective filter material 1801 and includes a strap 1802 for holding the mask 1800 to the wearer's face by looping over the ears or around the back of neck and head. The mask 1800 has an opening 1803, with a perimeter 1804, through the protective filter material 1801. The opening 1803 allows a collection filter assembly 1815 to be attached to the outside of mask 1800 with a bond sealing the collection filter assembly around the perimeter of the opening.

Collection filter assembly 1815 is made up of interior protective layer 1806 with interior protective layer tab 1805, collection filter 1808 with collection filter tab 1807, exterior protective layer 1810 with exterior protective layer tab 1809, usage indicator 1814, and support frame 1813 with opening 1812 and support frame tab 1811. When wearing the mask 1800, exhaled and inhaled air can pass through the protective filter material 1801 and through the opening 1803 and collection filter assembly 1815, as well as by or through usage indicator 1814. In this exemplary embodiment, the resistance (e.g., pressure drop) for air flowing through the collection filter assembly 1815 will generally be less than equal to the resistance (e.g., pressure drop) for air flowing through an equivalent surface area of the protective filter material 1801. This configuration allows for increased airflow through the collection filter assembly 1815 and therefor increased collection of target particles onto the collection filter assembly 1815 while providing easy access to the collection filter assembly 1815 by health care workers. The collection filter assembly 1815 may be made up of all or a portion of the layers of material used in the protective filter material 1801. In this embodiment specifically, the collection filter 1808 may be constructed from the internal filter material that is primary filter layer within the protective filter material 1801 of many N95 masks.

The collection filter 1808 can be selected from a range of filter materials that will be known to those skilled in the art, including but not limited to: non-woven, woven, membrane, fiber, depth, electret, hydrophobic, or hydrophilic filtering materials. The frame 1813 can be selected from a range of materials including thin, flexible plastic sheet material, hard plastics, rubbers, woven fabric, non-woven fabric, and membrane materials. The frame 1813 offers structural integrity to the collection filter assembly 1815, while limiting the potential for damage or contamination of collection filter 1808 during use and during removal from the mask.

The usage indicator 1814 utilizes a pH indicator or moisture indicator to provide a visible signal to the user, health care worker, or laboratory worker that the mask has been worn for a sufficient time period. Exhaled carbon dioxide is readily soluble in water—forming carbonic acid. When in a high humidity environment carbonic acid is formed in water captured onto surfaces and within absorbent materials. To produce usage indicator 1814, standard pH indicators, like those used in pH indicator strips, are impregnated onto a solid substrate or into a porous substrate. Said substrate is integrated into the collection filter assembly 1815 as part of a filter, protective layer, or support frame 1813. The usage indicator 1814 can be designed to be visible through opening 1812 with or without a clear covering over the indicator. The usage indicator 1814 may also be on the inside surface of the collection filter assembly 1815, such that it is only visible after removal from mask 1800 or by removing the mask from the wearer and looking through opening 1803. Further, the indicator may also be contained within the collection filter assembly 1815, such that it is only visible after removal from the mask 1800 and separation of the layers.

The interior protective layer 1806 with interior protective layer tab 1805, collection filter 1808 with collection filter tab 1807, exterior protective layer 1810 with exterior protective layer tab 1809, usage indicator 1814, and support frame 1813 with opening 1812 and support frame tab 1811 are bonded together to form the collection filter assembly 1815. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1815 will be removed from the mask 1800 for placement into a sample tube for particle extraction, or the bond between the layers may be removable, or they may be bonded with a tear-away portion, to allow for separation of the layers. In this way, the collection filter 1808 may be placed into a sample tube for transport to a laboratory or for immediate extraction.

The collection filter 1808 may be transported dry or wet. For dry transport a desiccant pack may be included in the transport kit to assist the filters in drying and as such helping maintain certain target materials. Alternatively, a liquid buffer may be supplied for the user to add to the sample tube along with the sample. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Similar to the above, the collection filter assembly 1815 is bonded to the border 1804 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1815 and the border 1804 must be airtight and removable. In this way, when worn, a large amount of exhaled and inhaled air will pass through the collection filter assembly 1815 layers and when the sampling period is complete the entire collection filter assembly may be easily removed from the mask 1800.

A tab on the collection filter assembly 1815 allow for the health care worker, wearers of the mask, or other persons to easily remove the collection filter assembly 1815 from the mask 1800. The person removing the collection filter assembly 1815 will generally be expected to have gloved hands and will place one hand onto the top of the nose area of the mask 1800 and grasp a combination or one of the tabs with the other hand and pull down to remove the collection filter assembly 1815. Other means of removing the collection filter assembly 1815, such as pulling up on the tabs, which in this case are located at the bottom of the collection filter assembly 1815, will be apparent to those known in the art. Because collection filter assembly 1815 can be easily removed from outside the mask, the potential for contamination of the health care worker or cross contamination of other samples is minimized.

After removal of the collection filter assembly 1815, target particles captured in the entire collection filter assembly may be extracted by placing the collection filter assembly 1815 into a sample tube or other container and vortexing, shaking, sonicating, rocking, or with wet foam elution. Alternatively, dependent on the design of the collection filter assembly 1815, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1815 has a particle removal efficiency similar to that of protective filter material 1801 and a resistance (e.g., pressure drop) generally less than or equal to the resistance (e.g., pressure drop) of an equivalent surface area of protective filter material 1801. In this way, the volume of air passing through the collection filter assembly 1815 is similar to that passing through an equivalent surface area of protective filter material 1801 and the amount of target captured per surface area is maximized. Further, this ensures that the wearer of the mask 1800 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

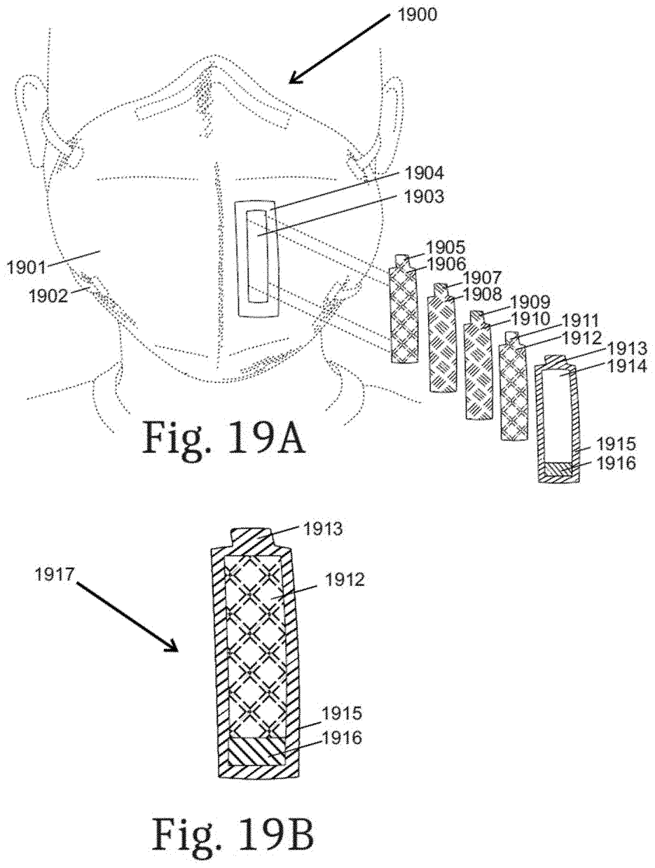

FIGS. 19A and 19B show a protective mask 1900 with a five-layer collection filter assembly 1917 with a usage indicator 1916 and support frame 1915, according to an exemplary embodiment of the present subject disclosure. The protective mask 1900 has a protective filter material 1901 and includes a strap 1902 for holding the mask 1900 to the wearer's face by looping over the ears or around the back of neck and head. The mask 1900 has an opening 1903, with a perimeter 1904, through the protective filter material 1901. The opening 1903 allows a collection filter assembly 1917 to be attached to the outside of mask 1900 with a bond sealing the collection filter assembly around the perimeter of the opening.

Collection filter assembly 1917 is made up of interior protective layer 1906 with interior protective layer tab 1905, source collection filter 1908 with source collection filter tab 1907, receptor collection filter 1910 with receptor collection filter tab 1909, exterior protective layer 1912 with exterior protective layer tab 1911, usage indicator 1916, and support frame 1915 with opening 1914 and support frame tab 1913. When wearing the mask 1900, exhaled and inhaled air can pass through the protective filter material 1901 and through the opening 1903 and collection filter assembly 1917, as well as by or through usage indicator 1916. In this exemplary embodiment, the resistance (e.g., pressure drop) for air flowing through the collection filter assembly 1917 will generally be less than equal to the resistance (e.g., pressure drop) for air flowing through an equivalent surface area of the protective filter material 1901. This configuration allows for increased airflow through the collection filter assembly 1917 and therefor increased collection of target particles onto the collection filter assembly 1917 while providing easy access to the collection filter assembly 1917 by health care workers. The collection filter assembly 1917 may be made up of all or a portion of the layers of material used in the protective filter material 1901. In this embodiment specifically, the source collection filter 1908 and receptor collection filter 1910 may be constructed from the internal filter material that is primary filter layer within the protective filter material of many N95 masks. Further interior protective layer 1906 and exterior protective layer 1912 may be constructed from the cover web material that is found within the protective filter material of many N95 masks.

The source collection filter 1908 and receptor collection filter 1910 can be selected from a range of filter materials that will be known to those skilled in the art, including but not limited to: non-woven, woven, membrane, fiber, depth, electret, hydrophobic, or hydrophilic filtering materials. Further, source collection filter 1908 and receptor collection filter 1910 may both be constructed from the same material or they may be constructed from different materials with different properties. The frame 1915 can be selected from a range of materials including thin, flexible plastic sheet material, hard plastics, rubbers, woven fabric, non-woven fabric, and membrane materials. The frame 1915 offers structural integrity to the collection filter assembly 1917, while limiting the potential for damage to the source collection filter 1908 and receptor collection filter 1910 during use and during removal from the mask.

The usage indicator 1916 utilizes a pH indicator or moisture indicator to provide a visible signal to the user, health care worker, or laboratory worker that the mask has been worn for a sufficient time period. Exhaled carbon dioxide is readily soluble in water—forming carbonic acid. When in a high humidity environment carbonic acid is formed in water captured onto surfaces and within absorbent materials. To produce usage indicator 1916, standard pH indicators, like those used in pH indicator strips, are impregnated onto a solid substrate or into a porous substrate. Said substrate is integrated into the collection filter assembly 1917 as part of a filter, protective layer, or support frame 1915. The usage indicator 1916 can be designed to be visible through opening 1914 with or without a clear covering over the indicator. The usage indicator 1916 may also be on the inside surface of the collection filter assembly 1917, such that it is only visible after removal from mask 1900 or by removing the mask from the wearer and looking through opening 1903. Further, the indicator may also be contained within the collection filter assembly 1917, such that it is only visible after removal from the mask 1900 and separation of the layers.

Interior protective layer 1906 with interior protective layer tab 1905, source collection filter 1908 with source collection filter tab 1907, receptor collection filter 1910 with receptor collection filter tab 1909, exterior protective layer 1912 with exterior protective layer tab 1911, and support frame 1915 with opening 1914 and support frame tab 1913 are bonded together to form the collection filter assembly 1917. They may be bonded to each other using any of a number of approaches that will be known to those skilled in the art, including but not limited to: adhesive, glue, tape, solvent bonding, ultrasonic weld, friction weld, laser welding, thermal bonding, or heat laminating. The bond between the layers may be permanent, wherein the entire collection filter assembly 1917 will be removed from the mask 1900 for placement into a sample tube for particle extraction, or the bond between the layers may be removable, or they may be bonded with a tear-away portion, to allow for separation of the layers. In this way, the frame 1915, interior protective layer tab 1905, and exterior protective layer 1912 may be discarded and source collection filter 1908 and receptor collection filter 1910 may be placed into a single sample tube or two separate sample tubes for transport to a laboratory or for immediate extraction.

The source collection filter 1908 and receptor collection filter 1910 may be transported dry or wet. For dry transport a desiccant pack may be included in the transport kit to assist the filters in drying and as such helping maintain certain target materials. Alternatively, a liquid buffer may be supplied for the user to add to the sample tube along with the sample. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Similar to the above, the collection filter assembly 1917 is bonded to the border 1904 using any of a number of approaches that will be known to those skilled in the art, including but not limited to: adhesive, glue, tape, solvent bonding, ultrasonic weld, friction weld, laser welding, thermal bonding, or heat laminating. The bond between the collection filter assembly 1917 and the border 1904 must be airtight and removable. In this way, when worn, a large amount of exhaled and inhaled air will pass through the collection filter assembly 1917 layers and when the sampling period is complete the entire collection filter assembly may be easily removed from the mask 1900.

A tab on the collection filter assembly 1917 allow for the health care worker, wearers of the mask, or other persons to easily remove the collection filter assembly 1917 from the mask 1900. The tabs may be bonded to each other or separated in a variety of ways to facilitate removal of the collection filter assembly 1917 from the mask 1900 and to facilitate separation into layers. The person removing the collection filter assembly 1917 will generally be expected to have gloved hands and will place one hand onto the top of the nose area of the mask 1900 and grasp a combination or one of the tabs with the other hand and pull down to remove the collection filter assembly 1917. Other means of removing the collection filter assembly 1917, such as pulling up on the tabs, which in this case are located at the bottom of the collection filter assembly 1917, will be apparent to those known in the art. Because collection filter assembly 1917 can be easily removed from outside the mask, the potential for contamination of the health care worker or cross contamination of other samples is minimized.

After removal of the collection filter assembly 1917, target particles captured in the entire collection filter assembly may be extracted by placing the collection filter assembly 1917 into a sample tube or other container and vortexing, shaking, sonicating, rocking, or with wet foam elution. Alternatively, dependent on the design of the collection filter assembly 1917, it may be separated using tabs on the components and gloved hands, forceps or other tools and collection filters within the assembly may be placed into sample container and shipped or transported to a lab for extraction or extraction may be performed in the field.

It should be noted that the material in the collection filter assembly 1917 has a particle removal efficiency similar to that of protective filter material 1901 and a resistance (e.g., pressure drop) generally less than or equal to the resistance (e.g., pressure drop) of an equivalent surface area of protective filter material 1901. In this way, the volume of air passing through the collection filter assembly 1917 is similar to that passing through an equivalent surface area of protective filter material 1901 and the amount of target captured per surface area is maximized. Further, this ensures that the wearer of the mask 1900 is protected at a similar level to protection provided by a standard protective mask of similar design.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

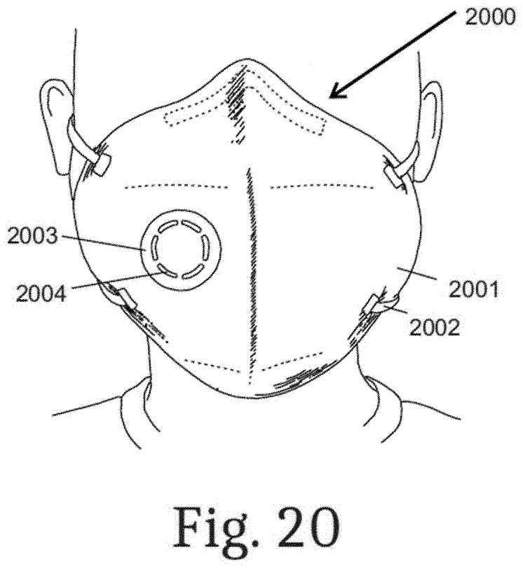

FIG. 20 shows an exemplary embodiment of a protective mask 2000 with a single cartridge-type collection filter assembly 2003. The mask 2000 has and protective filter material 2001 and includes a strap 2002 for holding the mask 2000 to the wearer's face by looping over the ears or around the back of neck and head. The mask 2000 has an opening, with a perimeter, through the protective filter material 2001. The opening allows a collection filter assembly 2003 to be attached to the outside of the mask 2000, for easy removal, while maximizing the flow of air through an internal filter in the collection filter assembly 2003. Collection filter assembly 2003 has vent openings 2004 for allowing flow through the assembly. Placement of collection filter assembly 2003 on the outside of mask 2000 allows health care workers to easily remove the collection filter assembly 2003 and then replace it with a new collection filter assembly 2003 or with a cap to cover the opening in mask 2000. This is helpful in that it allows for sample collection in a health care setting without having potentially infected individuals removing their mask to allow for sample collection. In this way, the entire time that the collection filter assembly 2003 is off of the mask, exposing the opening through the protective layer 2001, is most likely significantly under 1 minute. This shortened time period significantly reduces the potential for infection of health care workers taking samples and also reduces the time required to collect samples from patients.

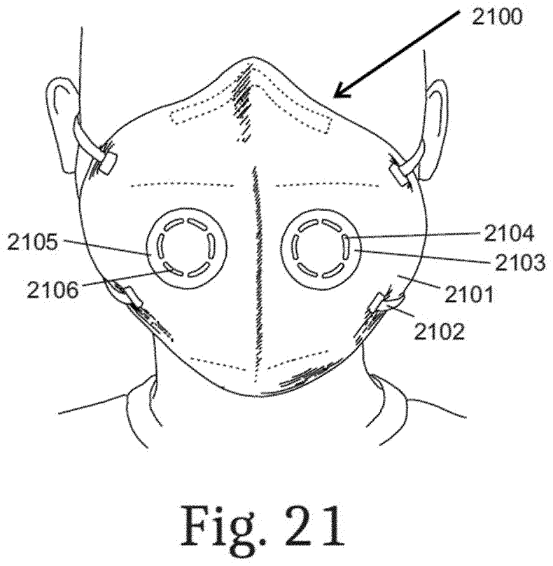

FIG. 21 shows an exemplary embodiment of a protective mask 2100 with two cartridge-type collection filter assemblies 2103 and 2105. The mask 2100 has and protective filter material 2101 and includes a strap 2102 for holding the mask 2100 to the wearer's face by looping over the ears or around the back of neck and head. The mask 2100 has two openings, each with a perimeter, through the protective filter material 2101. The openings allow collection filter assemblies 2103 and 2105 to be attached to the outside of the mask 2100, for easy removal, while maximizing the flow of air through an internal filter in each of collection filter assemblies 2103 and 2105. Collection filter assemblies 2103 and 2105 each have vent openings 2104 and 2106, respectively, for allowing flow through the assembly. Placement of collection filter assemblies 2103 and 2105 on the outside of mask 2100 allows health care workers to easily remove collection filter assemblies 2103 and 2105 and then replace each with a new collection filter assembly 2103 or with caps to cover the openings in mask 2100. This is important in that it allows for sample collection in a health care setting without having potentially infected individuals removing their mask to allow for sample collection. In this way, the entire time that each of collection filter assemblies 2103 and 2105 is off the mask, exposing the opening through the protective layer 2101, is most likely significantly under 1 minute. This shortened time period significantly reduces the potential for infection of health care workers taking samples and also reduces the time required to collect samples from patients. The use of two collection filter assemblies 2103 and 2105 allows on to be configured for collection of exhaled air only (source filter), using a check valve, and one to be configured for collection of inhaled air only (receptor filter), using a check valve. This configuration enables determination of whether target particles originated from the wearer or from the environment around the wearer.

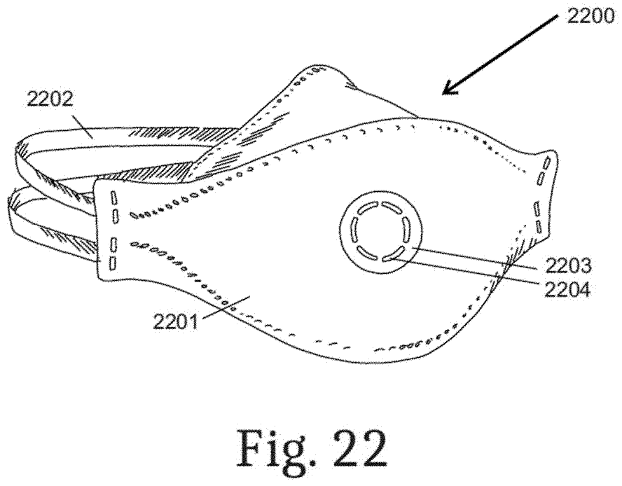

FIG. 22 shows an exemplary embodiment of an alternative fold-flat style protective mask 2200 design with a single cartridge-type collection filter assembly 2203. The mask 2200 has and protective filter material 2201 and includes a strap 2202 for holding the mask 2200 to the wearer's face by looping over the ears or around the back of neck and head. Mask 2200 is the same design as shown in FIG. 20 but uses an alternative style of fold-flat mask. Collection filter assembly 2203 can be used on any number of protective mask designs as would be conceivable by persons having ordinary skill in the art in light of this disclosure. The mask 2200 has an opening, with a perimeter, through the protective filter material 2201. The opening allows a collection filter assembly 2203 to be attached to the outside of the mask 2200, for easy removal, while maximizing the flow of air through an internal filter in the collection filter assembly 2203. Collection filter assembly 2203 has vent openings 2204 for allowing flow through the assembly. Placement of collection filter assembly 2203 on the outside of mask 2200 allows health care workers to easily remove the collection filter assembly 2203 and then replace it with a new collection filter assembly 2203 or with a cap to cover the opening in mask 2200. This is important in that it allows for sample collection in a health care setting without having potentially infected individuals removing their mask to allow for sample collection. In this way, the entire time that the collection filter assembly 2203 is off of the mask, exposing the opening through the protective layer 2201, is most likely significantly under 1 minute. This shortened time period significantly reduces the potential for infection of health care workers taking samples and also reduces the time required to collect samples from patients.

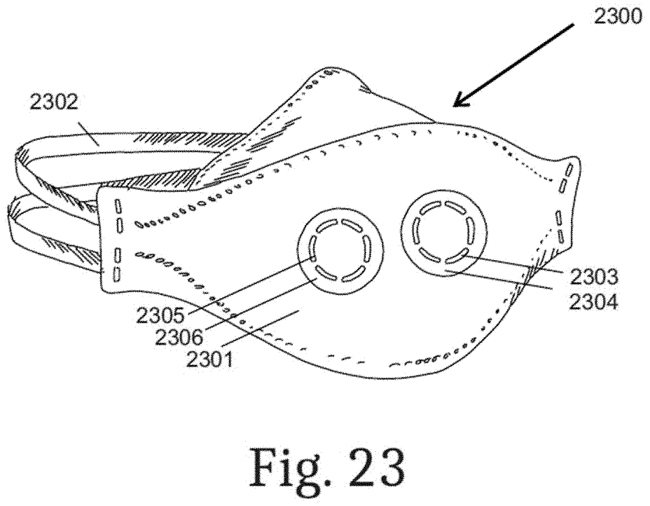

FIG. 23 shows an exemplary embodiment an alternative fold-flat style protective mask 2300 design with two cartridge-type collection filter assemblies 2303 and 2305. The mask 2300 has and protective filter material 2301 and includes a strap 2302 for holding the mask 2300 to the wearer's face by looping over the ears or around the back of neck and head. Mask 2300 is the same design as shown in FIG. 21 but uses an alternative style of fold-flat mask. Collection filter assembly 2303 can be used on any number of protective mask designs as would be conceivable by persons having ordinary skill in the art in light of this disclosure. The mask 2300 has two openings, each with a perimeter, through the protective filter material 2301. The openings allow collection filter assemblies 2303 and 2305 to be attached to the outside of the mask 2300, for easy removal, while maximizing the flow of air through an internal filter in each of collection filter assemblies 2303 and 2305. Collection filter assemblies 2303 and 2305 each have vent openings 2304 and 2016, respectively, for allowing flow through the assembly. Placement of collection filter assemblies 2303 and 2305 on the outside of mask 2300 allows health care workers to easily remove collection filter assemblies 2303 and 2305 and then replace each with a new collection filter assembly 2303 or with caps to cover the openings in mask 2300. This is important in that it allows for sample collection in a health care setting without having potentially infected individuals removing their mask to allow for sample collection. In this way, the entire time that each of collection filter assemblies 2303 and 2305 is off the mask, exposing the opening through the protective layer 2301, is most likely significantly under 1 minute. This shortened time period significantly reduces the potential for infection of health care workers taking samples and also reduces the time required to collect samples from patients. The use of two collection filter assemblies 2303 and 2305 allows on to be configured for collection of exhaled air only (source filter), using a check valve, and one to be configured for collection of inhaled air only (receptor filter), using a check valve. This configuration enables determination of whether target particles originated from the wearer or from the environment around the wearer.

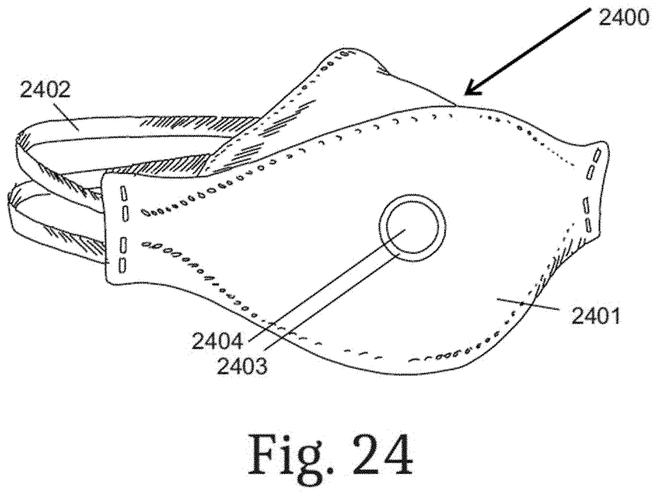

FIG. 24 shows an exemplary embodiment of an alternative fold-flat style protective mask 2400 design with a single cartridge-type collection filter assembly 2403 without a plastic cover. The mask 2400 has and protective filter material 2401 and includes a strap 2402 for holding the mask 2400 to the wearer's face by looping over the ears or around the back of neck and head. Mask 2400 is the same design as shown in FIG. 22 but uses an open collection filter assembly 2403 with no cover. Collection filter assembly 2403 can include a combination of protective layers of cover web and one or more layers of collection filters. One design includes a source collection filter on the interior side and a receptor collection filter on the exterior side. This enables identification of the source of collected material, as coming from the wearer or the environment around the wearer. The mask 2400 has an opening, with a perimeter, through the protective filter material 2401. The opening allows a collection filter assembly 2403 to be attached to the outside of the mask 2400, for easy removal, while maximizing the flow of air through an internal filter in the collection filter assembly 2403. Placement of collection filter assembly 2403 on the outside of mask 2400 allows health care workers to easily remove the collection filter assembly 2403 and then replace it with a new collection filter assembly 2403 or with a cap to cover the opening in mask 2400. This is important in that it allows for sample collection in a health care setting without having potentially infected individuals removing their mask to allow for sample collection. In this way, the entire time that the collection filter assembly 2403 is off of the mask, exposing the opening through the protective layer 2401, is most likely significantly under 1 minute. This shortened time period significantly reduces the potential for infection of health care workers taking samples and also reduces the time required to collect samples from patients.

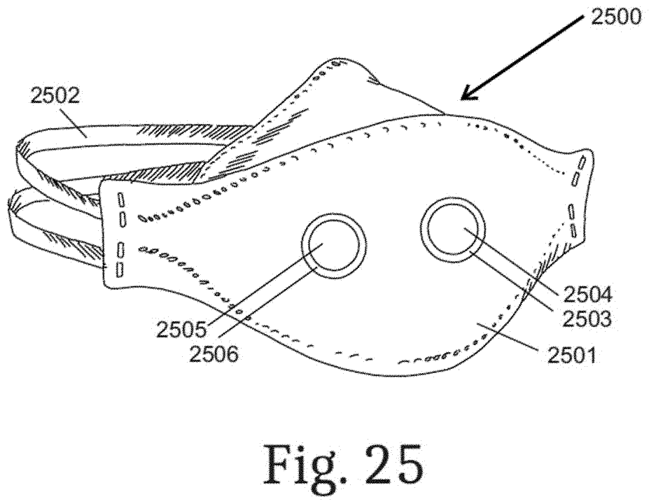

FIG. 25 shows an exemplary embodiment of an alternative fold-flat style protective mask 2500 design with two cartridge-type collection filter assemblies 2503 and 2505 without plastic covers. The mask 2500 has and protective filter material 2501 and includes a strap 2502 for holding the mask 2500 to the wearer's face by looping over the ears or around the back of neck and head. Mask 2500 is the same design as shown in FIG. 23 but uses open collection filter assemblies 2503 and 2505 with no cover. Collection filter assemblies 2503 and 2505 can include a combination of protective layers of cover web and one or more layers of collection filters. One design includes a source collection filter on the interior side and a receptor collection filter on the exterior side. This enables identification of the source of collected material, as coming from the wearer or the environment around the wearer. The use of two collection filter assemblies 2503 and 2505 allows for sample collection for two different sample processing or analysis techniques or for collection of samples at two different time points. Collection filter assembly 2503 can be used on any number of protective mask designs as would be conceivable by persons having ordinary skill in the art in light of this disclosure. The mask 2500 has two openings, each with a perimeter, through the protective filter material 2501. The openings allow collection filter assemblies 2503 and 2505 to be attached to the outside of the mask 2500, for easy removal, while maximizing the flow of air through an internal filter in each of collection filter assemblies 2503 and 2505. Placement of collection filter assemblies 2503 and 2505 on the outside of mask 2500 allows health care workers to easily remove collection filter assemblies 2503 and 2505 and then replace each with a new collection filter assembly 2503 or with caps to cover the openings in mask 2500. This is important in that it allows for sample collection in a health care setting without having potentially infected individuals removing their mask to allow for sample collection. In this way, the entire time that each of collection filter assemblies 2503 and 2505 is off the mask, exposing the opening through the protective layer 2501, is most likely significantly under 1 minute. This shortened time period significantly reduces the potential for infection of health care workers taking samples and also reduces the time required to collect samples from patients. The use of two collection filter assemblies 2503 and 2505 allows on to be configured for collection of exhaled air only (source filter), using a check valve, and one to be configured for collection of inhaled air only (receptor filter), using a check valve. This configuration enables determination of whether target particles originated from the wearer or from the environment around the wearer.

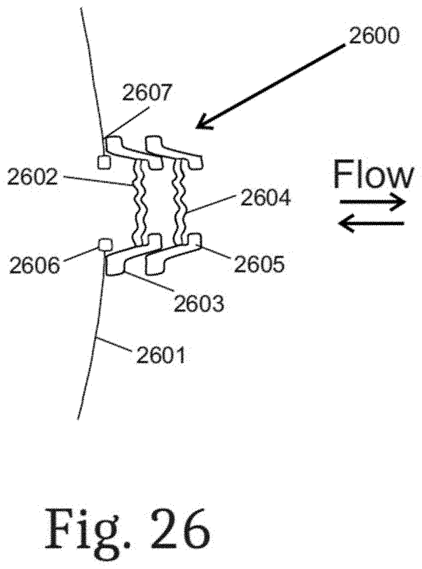

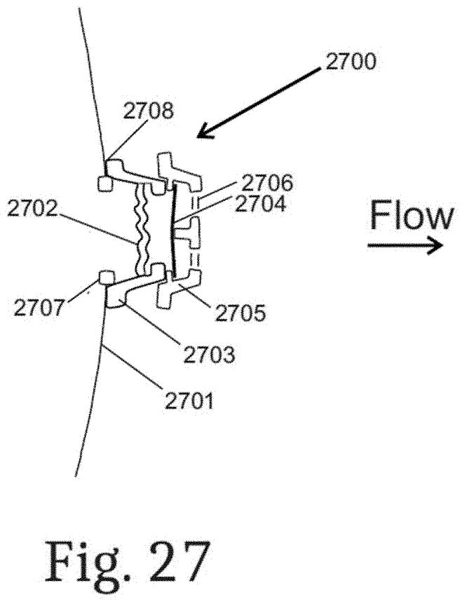

FIG. 26 shows an exemplary embodiment of a cartridge-type collection filter assembly 2600 with a source collection filter 2602 and receptor collection filter 2604. The collection filter assembly 2600 can be installed into protective masks like those shown in FIG. 20 through 25. The collection filter assembly 2600 is made up of a source filter cartridge 2603, with source collection filter 2602, and a receptor filter cartridge 2605, with receptor collection filter 2604, which are installed into a support ring 2606. Support ring 2606 is bonded to the mask protective layer 2601 and provides an air-tight seal at 2607 with source filter cartridge 2603.

Collection filter assembly 2600 allows for air flow both during wearer inhalation and exhalation, as shown by the flow arrows in FIG. 26. The source collection filter 2602 and receptor collection filter 2604 can be selected from a range of filter materials that will be known to those skilled in the art, including but not limited to: non-woven, woven, membrane, fiber, depth, electret, hydrophobic, or hydrophilic filtering materials. Further, source collection filter 2602 and receptor collection filter 2604 may both be constructed from the same material or they may be constructed from different materials with different properties.

The tables provided in FIG. 49 and FIG. 50 provide an example of how using source and receptor filters in series, as is shown in FIG. 26, allows for data to be used to determine if the detected particles originated from the wearer or from the environment around the wearer. Assuming a 77.7% efficiency for each filter layer, and in the case of an infected wearer that is shedding virus, the interior source collection filter 2602 will collect a minimum of approximately 4.5 times more target particles than the receptor collection filter 2604. This difference is sufficient to allow for a determination as to whether target particles originated from an infected wearer or a contaminated environment. Further, as the efficiency of each layer is increased the ratio of target particles collected by each filter increases. As an example, two 90% efficiency filters will provide a minimum of approximately 10 times more particles on source collection filter 2602 if the wearer is contaminated or 10 times more on receptor collection filter 2604 if the wearer is in a contaminated environment. Finally, these estimated ratios are most likely lower than they will be in actual use cases because not all particles containing target material are 0.3 μm in diameter, but are rather distributed over a large particle size range, and, in general, particles larger than and smaller than 0.3 μm in diameter are captured with higher efficiency which will lead to larger ratios.

After the mask has been worn for a period of time enabling sufficient target particle collection, generally ranging from 5 minutes to as much as 8 hours, the source filter cartridge 2603 and receptor filter cartridge 2605 can be removed from the mask. After removal the cartridges can be placed into a sample tube, plastic bag, bottle or other transport container. In most cases, the transport container will double as the laboratory sample processing container as well. The source collection filter 2602 and receptor collection filter 2604 may be transported dry or wet. For dry transport a desiccant pack may be included in the transport kit to assist the filters in drying and as such helping maintain certain target materials. Alternatively, the source filter cartridge 2603 and receptor filter cartridge 2605 may be placed into separate sample tubes and a liquid buffer, supplied with the sample transport kit, may be added onto the filter within the cartridge in the sample tube prior to sealing for transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the source collection filter 2602 and receptor collection filter 2604 can be extracted from the filters by dispensing a lysis/elution buffer onto the filter, incubating at room temperature or an elevated temperature and then allowing the buffer to drip into the sample container by gravity or using centrifugation or vacuum source to more quickly pull the sample into the sample tube. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

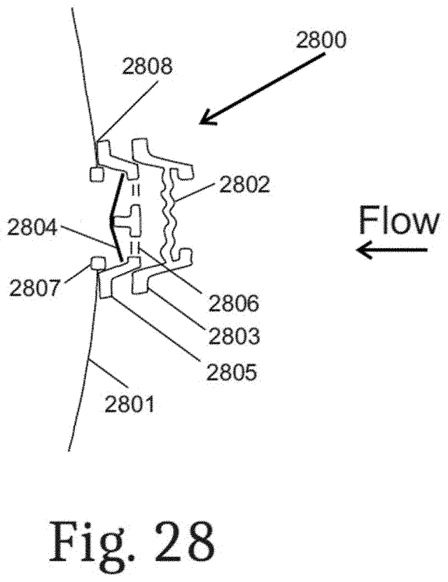

FIG. 27 shows an exemplary embodiment of a cartridge-type collection filter assembly 2700 with a source collection filter 2702 and check valve 2704. The collection filter assembly 2700 can be installed into protective masks like those shown in FIG. 20 through 25. Specifically, FIG. 21 and FIG. 25, show configurations with two cartridges installed, which can be used to allow for a source collection filter in one location and a receptor collection filter in the other location. The collection filter assembly 2700 is made up of a source filter cartridge 2703, with source collection filter 2702, and a check valve cartridge 2705 with check valve 2704. Source filter cartridge 2703 is installed into a support ring 2707 and check valve cartridge 2705 is installed into source filter cartridge 2702. Support ring 2707 is bonded to the mask protective layer 2701 and provides an air-tight seal at 2708 with source filter cartridge 2703.

Check valve 2704 opens during wearer exhalation to allow air to flow out through source filter 2702 and through opening 2706. During inhalation check valve 2704 closes, sealing off the flow path and not allowing exterior environmental air to pass through source filter 2702. By sealing during inhalation, the source filter 2702 only captures particles being exhaled by the wearer and, as such, provides a diagnostic of the wearers infectious state and potential as a spreader of viral or microbial pathogen. The source collection filter 2702 can be selected from a range of filter materials that will be known to those skilled in the art, including but not limited to: non-woven, woven, membrane, fiber, depth, electret, hydrophobic, or hydrophilic filtering materials.

After the mask has been worn for a period of time enabling sufficient target particle collection, generally ranging from 5 minutes to as much as 8 hours, the source filter cartridge 2703 can be removed from the mask and separated from the check valve cartridge 2705. After removal, the source filter cartridge 2703 can be placed into a sample tube, plastic bag, bottle or other transport container. In most cases, the transport container will double as the laboratory sample processing container as well. The source collection filter 2702 may be transported dry or wet. In an alternative embodiment the source collection filter 2702 may be removable from source filter cartridge 2703, so that it can be placed directly into a small sample transport container. For dry transport a desiccant pack may be included in the transport kit to assist the filters in drying and as such helping maintain certain target materials. Alternatively, the source filter cartridge 2703 may be placed into a sample tube and a liquid buffer, supplied with the sample transport kit, may be added onto the filter within the cartridge in the sample tube prior to sealing for transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the source collection filter 2702 can be extracted from the filters by dispensing a lysis/elution buffer onto the filter, incubating at room temperature or an elevated temperature and then allowing the buffer to drip into the sample container by gravity or using centrifugation or vacuum source to more quickly pull the sample into the sample tube. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

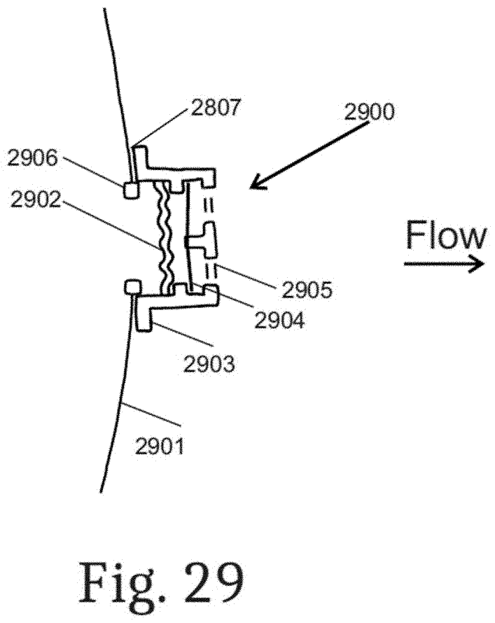

FIG. 28 shows an exemplary embodiment of a cartridge-type collection filter assembly 2800 with a receptor collection filter 2802 and check valve 2804. The collection filter assembly 2800 can be installed into protective masks like those shown in FIG. 20 through 25. Specifically, FIG. 21 and FIG. 25, show configurations with two cartridges installed, which can be used to allow for a source collection filter in one location and a receptor collection filter in the other location. The collection filter assembly 2800 is made up of a receptor filter cartridge 2803, with receptor collection filter 2802, and a check valve cartridge 2805 with check valve 2804. Check valve cartridge 2805 is installed onto a support ring 2807 and receptor filter cartridge 2803 is installed onto check valve cartridge 2805. Support ring 2807 is bonded to the mask protective layer 2801 and provides an air-tight seal at 2808 with check valve cartridge 2805.

Check valve 2804 opens during wearer inhalation to allow air to flow in through receptor filter 2802 and through opening 2806. During exhalation check valve 2804 closes, sealing off the flow path and not allowing the wearer's exhaled air to pass through receptor collection filter 2802. By sealing during exhalation, the receptor collection filter 2802 only captures particles being inhaled by the wearer and, as such, provides a diagnostic of the wearer's environment. The receptor collection filter 2802 can be selected from a range of filter materials that will be known to those skilled in the art, including but not limited to: non-woven, woven, membrane, fiber, depth, electret, hydrophobic, or hydrophilic filtering materials.

After the mask has been worn for a period of time enabling sufficient target particle collection, generally ranging from 5 minutes to as much as 8 hours, the receptor filter cartridge 2803 can be removed from the mask and separated from the check valve cartridge 2805. After removal, the receptor filter cartridge 2803 can be placed into a sample tube, plastic bag, bottle or other transport container. In most cases, the transport container will double as the laboratory sample processing container as well. The receptor collection filter 2802 may be transported dry or wet. In an alternative embodiment the receptor collection filter 2802 may be removable from receptor filter cartridge 2803, so that it can be placed directly into a small sample transport container. For dry transport a desiccant pack may be included in the transport kit to assist the filters in drying and as such helping maintain certain target materials. Alternatively, the receptor filter cartridge 2803 may be placed into a sample tube and a liquid buffer, supplied with the sample transport kit, may be added onto the filter within the cartridge in the sample tube prior to sealing for transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the receptor collection filter 2802 can be extracted from the filters by dispensing a lysis/elution buffer onto the filter, incubating at room temperature or an elevated temperature and then allowing the buffer to drip into the sample container by gravity or using centrifugation or vacuum source to more quickly pull the sample into the sample tube. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

FIG. 29 shows an exemplary embodiment of an alternative cartridge-type collection filter assembly with a source collection filter 2902 and check valve 2904 in a single source filter cartridge 2903. The collection filter assembly 2900 can be installed into protective masks like those shown in FIG. 20 through 25. Specifically, FIG. 21 and FIG. 25, show configurations with two cartridges installed, which can be used to allow for a source collection filter in one location and a receptor collection filter in the other location. The collection filter assembly 2900 is made up of a single source filter cartridge 2903 with source collection filter 2902 and a check valve 2904. Source filter cartridge 2903 is installed into a support ring 2906. Support ring 2906 is bonded to the mask protective layer 2901 and provides an air-tight seal at 2907 with source filter cartridge 2903.

Check valve 2904 opens during wearer exhalation to allow air to flow out through source filter 2902 and through opening 2905. During inhalation check valve 2904 closes, sealing off the flow path and not allowing exterior environmental air to pass through source filter 2902. By sealing during inhalation, the source filter 2902 only captures particles being exhaled by the wearer and, as such, provides a diagnostic of the wearers infectious state and potential as a spreader of viral or microbial pathogens. The source collection filter 2902 can be selected from a range of filter materials that will be known to those skilled in the art, including but not limited to: non-woven, woven, membrane, fiber, depth, electret, hydrophobic, or hydrophilic filtering materials.

After the mask has been worn for a period of time enabling sufficient target particle collection, generally ranging from 5 minutes to as much as 8 hours, the source filter cartridge 2903 can be removed from the mask. After removal, the source filter cartridge 2903 can be placed into a sample tube, plastic bag, bottle or other transport container. In most cases, the transport container will double as the laboratory sample processing container as well. The source collection filter 2902 may be transported dry or wet. In an alternative embodiment the source collection filter 2902 may be removable from source filter cartridge 2903, so that it can be placed directly into a small sample transport container. For dry transport a desiccant pack may be included in the transport kit to assist the filters in drying and as such helping maintain certain target materials. Alternatively, the source filter cartridge 2903 may be placed into a sample tube and a liquid buffer, supplied with the sample transport kit, may be added onto the filter within the cartridge in the sample tube prior to sealing for transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the source collection filter 2902 can be extracted from the filters by dispensing a lysis/elution buffer onto the filter, incubating at room temperature or an elevated temperature and then allowing the buffer to drip into the sample container by gravity or using centrifugation or vacuum source to more quickly pull the sample into the sample tube. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

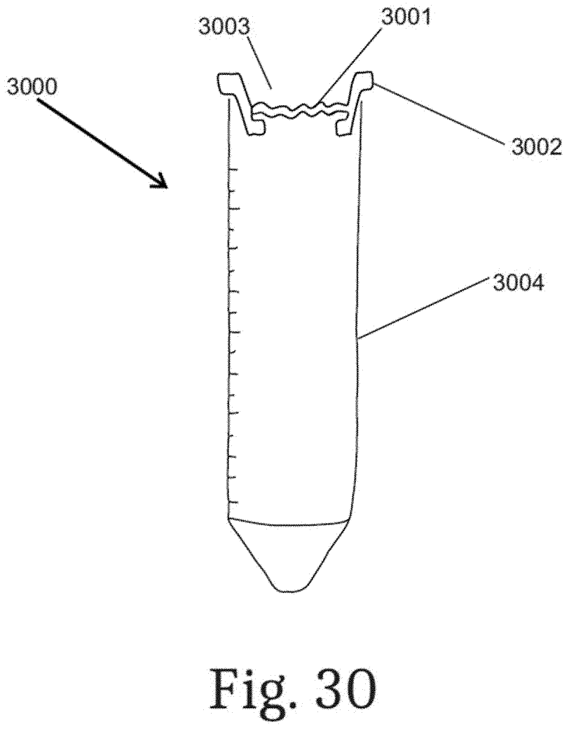

FIG. 30 shows an exemplary embodiment of a filter cartridge in a sample tube 3000. The filter cartridge 3002 holds a collection filter 3001 and is sized to allow it to fit into a sample tube 3004 or centrifuge tube. The filter cartridge 3002 can be installed into protective masks like those shown in FIG. 20 through 25 as part of the cartridge-type collection filter assemblies shown in FIG. 26 through 28. The filter cartridge 3002 can be used as a source filter cartridge, with a source collection filter, or as a receptor filter cartridge, with receptor collection filter.

After the mask has been worn for a period of time enabling sufficient target particle collection, generally ranging from 5 minutes to as much as 8 hours, the filter cartridge 3002 can be removed from the mask and placed into a sample tube 3004. The sample may then be transported to the lab in the sample tube 3004 or extracted and analyzed at the current location. The sample may be transported dry or wet. For dry transport a desiccant pack may be included in the transport kit to assist the filters in drying and as such helping maintain certain target materials. Alternatively, liquid buffer, supplied with the sample transport kit, may be dispensed into the filter cartridge 3002 top opening 3003 above the collection filter 3001 prior to transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the collection filter 3001 can be extracted from the filters by dispensing a lysis/elution buffer onto the filter, incubating at room temperature or an elevated temperature and then allowing the buffer to drip into the sample container by gravity or using centrifugation or vacuum source to more quickly pull the sample into the sample tube. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

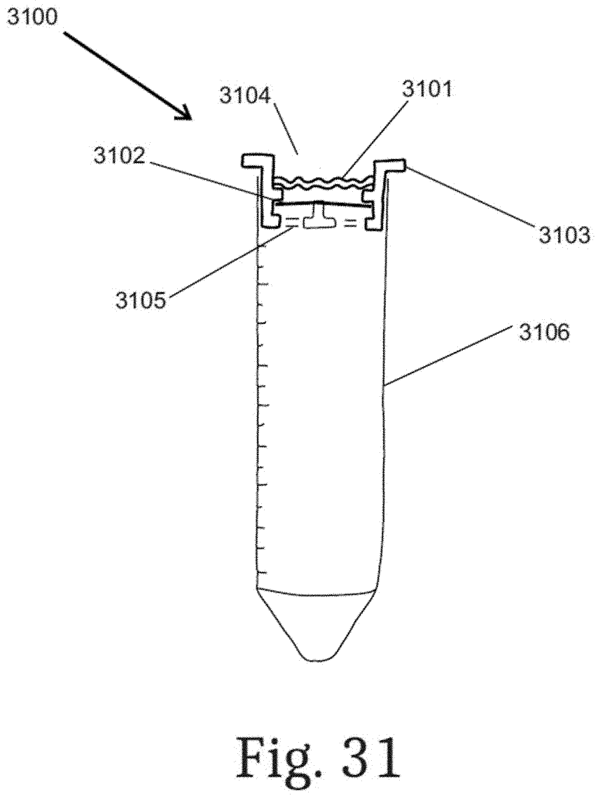

FIG. 31 shows an exemplary embodiment of an alternative filter cartridge in a sample tube 3106. The filter cartridge 3103 can be installed into protective masks like those shown in FIG. 20 through 25. The filter cartridge 3103 with collection filter 3101 and check valve 3102. The filter cartridge 3103 is primarily designed to serve as a source collection device with the cartridge on the outside of the mask, but could be reversed to also serve as a receptor filter cartridge. Check valve 3102 opens during wearer exhalation to allow air to flow out through collection filter 3101 and through opening 3105. During inhalation check valve 3102 closes, sealing off the flow path and not allowing exterior environmental air to pass through collection filter 3101. By sealing during inhalation, the collection filter 3101 only captures particles being exhaled by the wearer and, as such, provides a diagnostic of the wearers infectious state and potential as a spreader of viral or microbial pathogens. The collection filter 3101 can be selected from a range of filter materials that will be known to those skilled in the art, including but not limited to: non-woven, woven, membrane, fiber, depth, electret, hydrophobic, or hydrophilic filtering materials.

After the mask has been worn for a period of time enabling sufficient target particle collection, generally ranging from 5 minutes to as much as 8 hours, the filter cartridge 3103 can be removed from the mask. After removal, the filter cartridge 3103 can be placed into a sample tube 3106. In most cases, the transport container will double as the laboratory sample processing container as well. The collection filter 3101 may be transported dry or wet. In an alternative embodiment the collection filter 3101 may be removable from filter cartridge 3103, so that it can be placed directly into a small sample transport container. For dry transport a desiccant pack may be included in the transport kit to assist the filters in drying and as such helping maintain certain target materials. Alternatively, the filter cartridge 3103 may be placed into a sample tube and a liquid buffer, supplied with the sample transport kit, may be added onto the filter within the cartridge in the sample tube prior to sealing for transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the collection filter 3101 can be extracted from the filters by dispensing a lysis/elution buffer onto the filter, incubating at room temperature or an elevated temperature and then allowing the buffer to drip into the sample container by gravity or using centrifugation or vacuum source to more quickly pull the sample into the sample tube. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

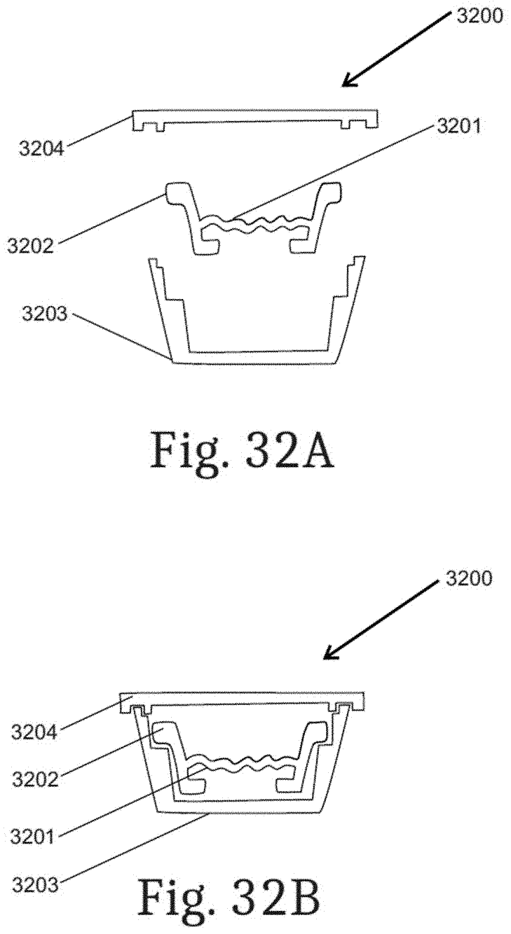

FIGS. 32A and 32B shows an exemplary embodiment of a filter cartridge in a small sample transport and extraction container 3200. The filter cartridge 3202 holds a collection filter 3201 and is supplied with a small sample container 3203 and lid 3204 that is used for transport and extraction. The filter cartridge 3202 can be installed into protective masks like those shown in FIG. 20 through 25 as part of the cartridge-type collection filter assemblies shown in FIG. 26 through 28. The filter cartridge 3202 can be used as a source filter cartridge, with a source collection filter, or as a receptor filter cartridge, with receptor collection filter.

After the mask has been worn for a period of time enabling sufficient target particle collection, generally ranging from 5 minutes to as much as 8 hours, the filter cartridge 3202 can be removed from the mask and placed into a sample container 3203 and contained with lid 3204. The sample may then be transported to the lab in the sample container 3203 or extracted and analyzed at the current location. The sample may be transported dry or wet. For dry transport a desiccant pack may be included in the transport kit to assist the filters in drying and as such helping maintain certain target materials. Alternatively, liquid buffer, supplied with the sample transport kit, may be dispensed onto collection filter 3201 prior to transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the collection filter 3201 can be extracted from the filters by dispensing a lysis/elution buffer onto the filter, incubating at room temperature or an elevated temperature and then allowing the buffer to drip into the sample container 3203 by gravity. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

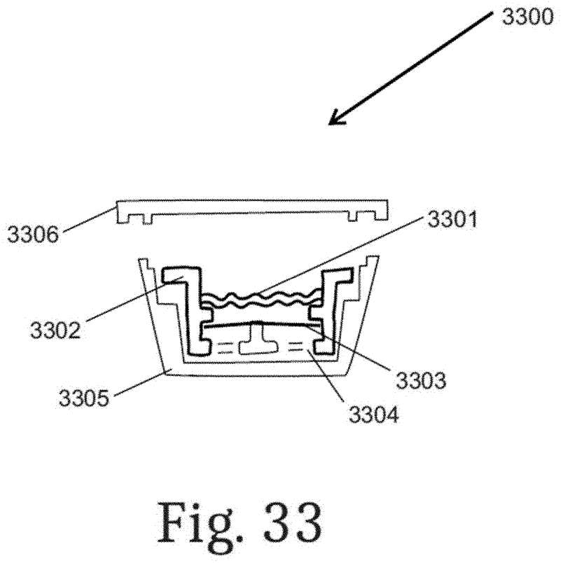

FIG. 33 shows an exemplary embodiment of an alternative cartridge-type collection filter assembly with a source filter 3302 and check valve 3304 in a small sample container 3305. The collection filter assembly 3300 can be installed into protective masks like those shown in FIG. 20 through 25. Specifically, FIG. 21 and FIG. 25, show configurations with two cartridges installed, which can be used to allow for a source collection filter in one location and a receptor collection filter in the other location. The collection filter assembly 3300 is made up of a single source filter cartridge 3303 with source collection filter 3302 and a check valve 3304. Source filter cartridge 3303 is installed into a support ring 3306. Support ring 3306 is bonded to the mask protective layer 3301 and provides an air-tight seal at 3307 with source filter cartridge 3303.

Check valve 3304 opens during wearer exhalation to allow air to flow out through source filter 3302 and through opening 3305. During inhalation check valve 3304 closes, sealing off the flow path and not allowing exterior environmental air to pass through source filter 3302. By sealing during inhalation, the source filter 3302 only captures particles being exhaled by the wearer and, as such, provides a diagnostic of the wearers infectious state and potential as a spreader of viral or microbial pathogens. The source collection filter 3302 can be selected from a range of filter materials that will be known to those skilled in the art, including but not limited to: non-woven, woven, membrane, fiber, depth, electret, hydrophobic, or hydrophilic filtering materials.

After the mask has been worn for a period of time enabling sufficient target particle collection, generally ranging from 5 minutes to as much as 8 hours, the source filter cartridge 3303 can be removed from the mask. After removal, the source filter cartridge 3303 can be placed into a sample container 3305. In most cases, the transport container will double as the laboratory sample processing container as well. The source collection filter 3302 may be transported dry or wet. In an alternative embodiment the source collection filter 3302 may be removable from source filter cartridge 3303, so that it can be placed directly into a small sample transport container. For dry transport a desiccant pack may be included in the transport kit to assist the filters in drying and as such helping maintain certain target materials. Alternatively, the source filter cartridge 3303 may be placed into a sample container 3305 and a liquid buffer, supplied with the sample transport kit, may be added onto the filter within the cartridge in the sample container 3305 prior to sealing for transport. Said liquid buffer stabilizing the target in the sample during transport. Viral transport media is well known in the art and any of a number of current transport medias or similar customer transport medias can be used for this purpose. Further, the addition of a lysis buffer may allow the sample to be lysed during transport, thus reducing the steps required in the laboratory upon arrival.

Target particles captured onto the source collection filter 3302 can be extracted from the filters by dispensing a lysis/elution buffer onto the filter, incubating at room temperature or an elevated temperature and then allowing the buffer to drip into the sample container 3305 by gravity. In this case, the lysis/elution buffer may be made up of one or more selected from buffers, salts, surfactants, detergents, chaotropic agents, RNase inhibitors, and other additives. Further, any number of lysis buffers that will be well known to those skilled in the art may be used for this step in the process. Further, vortexing, shaking, sonicating, rocking, wet foam elution, or other methods that will be well known to those skilled in the art may be used for this extraction of target particles from the collection filters.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

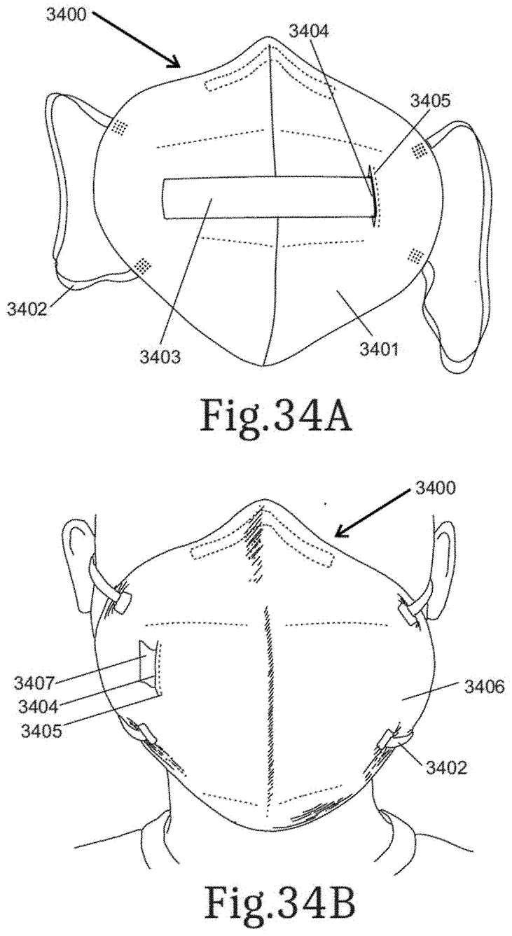

FIGS. 34A and 34B show a protective mask 3400 with a pull-through, horizontal strip collection substrate 3403, according to an exemplary embodiment of the present subject disclosure. The mask 3400 has a protective filter material with an interior face 3401 and an exterior face 3406 and includes a strap 3402 for holding the mask 3400 to the wearer's face by looping over the ears or around the back of neck and head. The mask 3400 has a collection substrate 3403 which is removably bonded to the interior face 3401 of the mask 3400 in a horizontal orientation with a tab 3407 through an opening 3404 in the protective filter material of the mask 3400. The opening 3404 is bonded closed with a removable adhesive or a tack weld line 3405.

Collection substrate 3403 can be selected from a range of filter materials that will be known to those skilled in the art, including but not limited to: non-woven, woven, membrane, fiber, depth, electret, hydrophobic, or hydrophilic filtering materials. Further the collection substrate 3403 may be constructed with an outside cover web or a flexible or semi-rigid inside structure that is designed to provide durability. Collection substrate 3403 is positioned in a horizontal orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 3400 will generally be worn for a time period between 5 minutes and 8 hours at which point the user or another person assisting the wearer can remove the collection substrate 3403 or the wearer can go to a clinical or testing facility to have the substrate removed by a healthcare worker. To remove the substrate the tab 3407 is grasped tightly with a gloved hand, forceps or other tool and is pulled strongly, to break the removable adhesive or weld line 3405, and the collection substrate 3403 is pulled out through the opening 3404. During this operation, the person removing the collection substrate 3403 may grasp the mask 3400, as necessary, to hold it steady. After removal, a small adhesive sticker can be placed over opening 3404 to eliminate air leakage through the opening.

Thus, when wearing the mask 3400, exhaled air can pass by the collection substrate 3403, and if it is manufactured from a permeable material, through it also, thus capturing viral or microbial particles of interest in the process. Placement of the collection substrate 3403 near the breathing zone allows for increased collection of target particles and design of the substrate to easily fit into a vortex tube enables more efficient elution into a small sample volume. Because tab 3407 passes through opening 3404 to the outside of mask 3400 it is easy for the health care worker or other person to remove the collection substrate 3403. Because collection substrate 3403 can be easily removed from outside the mask, the potential for contamination of the health care worker or cross contamination of other samples is minimized. After removal of the collection substrate 3403, target may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

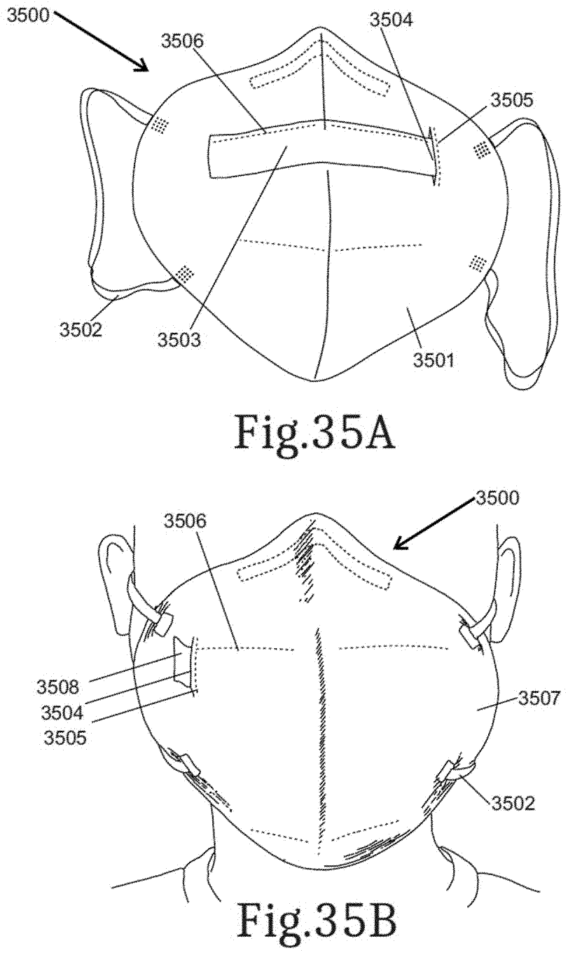

FIGS. 35A and 35B show a protective mask 3500 with an alternative configuration of a pull-through, horizontal strip collection substrate 3503 with a welded attachment to the mask, according to an exemplary embodiment of the present subject disclosure. The mask 3500 has a protective filter material with an interior face 3501 and an exterior face 3508 and includes a strap 3502 for holding the mask 3500 to the wearer's face by looping over the ears or around the back of neck and head. The mask 3500 has a collection substrate 3503 which is removably bonded using a weld line 3506 to the interior face 3501 of the mask 3500 with a tab 3508 through an opening 3504 in the protective filter material of the mask 3500. The opening 3504 is bonded closed with a removable adhesive or a tack weld line 3505.

Collection substrate 3503 can be selected from a range of filter materials that will be known to those skilled in the art, including but not limited to: non-woven, woven, membrane, fiber, depth, electret, hydrophobic, or hydrophilic filtering materials. Further the collection substrate 3503 may be constructed with an outside cover web or a flexible or semi-rigid inside structure that is designed to provide durability. Collection substrate 3503 is positioned in a horizontal orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 3500 will generally be worn for a time period between 5 minutes and 8 hours at which point the user or another person assisting the wearer can remove the collection substrate 3503 or the wearer can go to a clinical or testing facility to have the substrate removed by a healthcare worker. To remove the substrate the tab 3508 is grasped tightly with a gloved hand, forceps or other tool and is pulled strongly, to break the removable adhesive or weld line 3505, and the collection substrate 3503 is pulled out through the opening 3504. During this operation, the person removing the collection substrate 3503 may grasp the mask 3500, as necessary, to hold it steady. After removal, a small adhesive sticker can be placed over opening 3504 to eliminate air leakage through the opening.

Thus, when wearing the mask 3500, exhaled air can pass by the collection substrate 3503, and if it is manufactured from a permeable material, through it also, thus capturing viral or microbial particles of interest in the process. Placement of the collection substrate 3503 near the breathing zone allows for increased collection of target particles and design of the substrate to easily fit into a vortex tube enables more efficient elution into a small sample volume. Because tab 3508 passes through opening 3504 to the outside of mask 3500 it is easy for the health care worker or other person to remove the collection substrate 3503. Because collection substrate 3503 can be easily removed from outside the mask, the potential for contamination of the health care worker or cross contamination of other samples is minimized. After removal of the collection substrate 3503, target may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

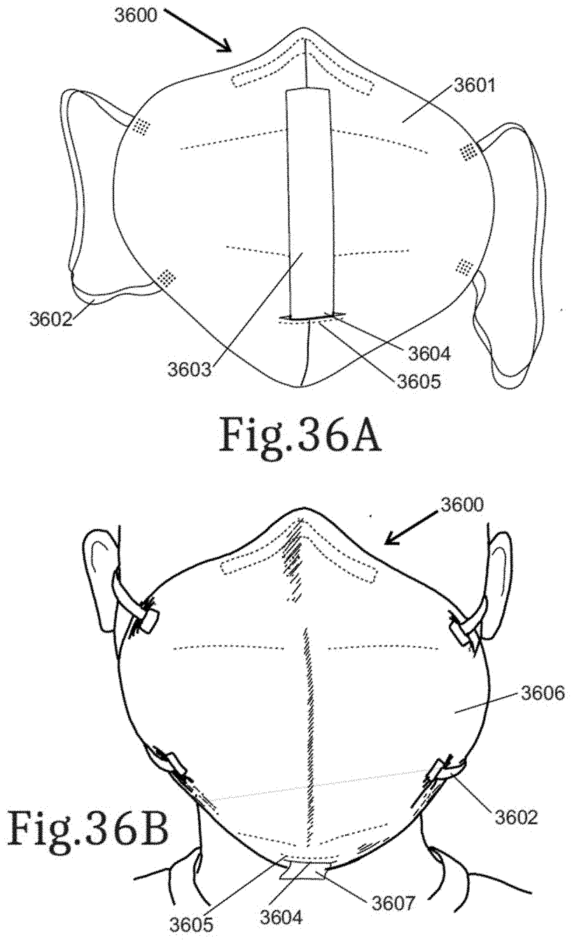

FIGS. 36A and 36B show a protective mask 3600 with a pull-through, vertical strip collection substrate 3603, according to an exemplary embodiment of the present subject disclosure. The mask 3600 has a protective filter material with an interior face 3601 and an exterior face 3606 and includes a strap 3602 for holding the mask 3600 to the wearer's face by looping over the ears or around the back of neck and head. The mask 3600 has a collection substrate 3603 which is removably bonded to the interior face 3601 of the mask 3600 in a vertical orientation with a tab 3607 through an opening 3604 in the protective filter material of the mask 3600. The opening 3604 is bonded closed with a removable adhesive or a tack weld line 3605.

Collection substrate 3603 can be selected from a range of filter materials that will be known to those skilled in the art, including but not limited to: non-woven, woven, membrane, fiber, depth, electret, hydrophobic, or hydrophilic filtering materials. Further the collection substrate 3603 may be constructed with an outside cover web or a flexible or semi-rigid inside structure that is designed to provide durability. Collection substrate 3603 is positioned in a vertical orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 3600 will generally be worn for a time period between 5 minutes and 8 hours at which point the user or another person assisting the wearer can remove the collection substrate 3603 or the wearer can go to a clinical or testing facility to have the substrate removed by a healthcare worker. To remove the substrate the tab 3607 is grasped tightly with a gloved hand, forceps or other tool and is pulled strongly, to break the removable adhesive or weld line 3605, and the collection substrate 3603 is pulled out through the opening 3604. During this operation, the person removing the collection substrate 3603 may grasp the mask 3600, as necessary, to hold it steady. After removal, a small adhesive sticker can be placed over opening 3604 to eliminate air leakage through the opening.

Thus, when wearing the mask 3600, exhaled air can pass by the collection substrate 3603, and if it is manufactured from a permeable material, through it also, thus capturing viral or microbial particles of interest in the process. Placement of the collection substrate 3603 near the breathing zone allows for increased collection of target particles and design of the substrate to easily fit into a vortex tube enables more efficient elution into a small sample volume. Because tab 3607 passes through opening 3604 to the outside of mask 3600 it is easy for the health care worker or other person to remove the collection substrate 3603. Because collection substrate 3603 can be easily removed from outside the mask, the potential for contamination of the health care worker or cross contamination of other samples is minimized. After removal of the collection substrate 3603, target may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

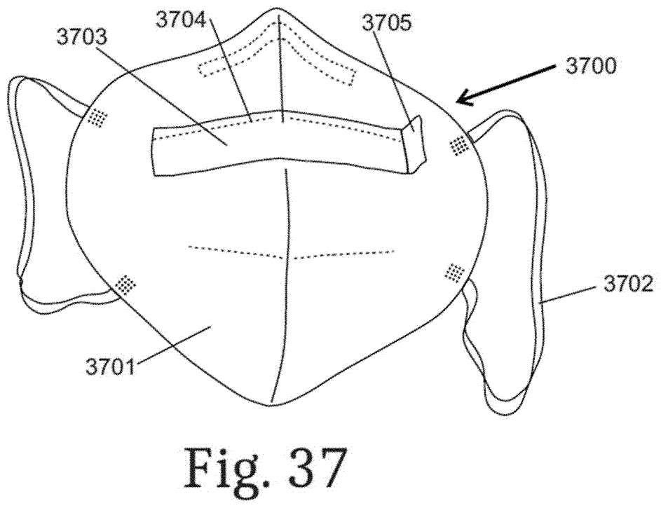

FIG. 37 shows a protective mask 3700 with horizontal strip collection substrate 3703 with a weld line 3704 attachment to the mask, according to an exemplary embodiment of the present subject disclosure. The mask 3700 has a protective filter material with an interior face 3701 and a strap 3702 for holding the mask 3700 to the wearer's face by looping over the ears or around the back of neck and head. The mask 3700 has a collection substrate 3703 which is removably bonded using a weld line 3704 to the interior face 3701 of the mask 3700.

Collection substrate 3703 can be selected from a range of filter materials that will be known to those skilled in the art, including but not limited to: non-woven, woven, membrane, fiber, depth, electret, hydrophobic, or hydrophilic filtering materials. Further the collection substrate 3703 may be constructed with an outside cover web or a flexible or semi-rigid inside structure that is designed to provide durability. Collection substrate 3703 is positioned in a horizontal orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 3700 will generally be worn for a time period between 5 minutes and 8 hours at which point the user or another person assisting the wearer can remove the collection substrate 3703 or the wearer can go to a clinical or testing facility to have the substrate removed by a healthcare worker. To remove the substrate, mask 3700 can first be removed or the tab 3705 can be located near the exterior of the mask 3700, where it can be grasped by the worker. The tab 3705 is grasped tightly with a gloved hand, forceps or other tool and is pulled strongly, to break the weld line 3705, and the collection substrate 3703 is pulled away from mask 3700. During this operation, the person removing the collection substrate 3703 may grasp the mask 3700, as necessary, to hold it steady.

When wearing the mask 3700, exhaled air can pass by the collection substrate 3703, and if it is manufactured from a permeable material, through it also, thus capturing viral or microbial particles of interest in the process. Placement of the collection substrate 3703 near the breathing zone allows for increased collection of target particles and design of the substrate to easily fit into a vortex tube enables more efficient elution into a small sample volume. In one design configuration, tab 3705 is located near the outside edge of mask 3700 so it is easy for the health care worker or other person to remove the collection substrate 3703. Because collection substrate 3703 can be easily removed from outside the mask, the potential for contamination of the health care worker or cross contamination of other samples is minimized. After removal of the collection substrate 3703, target may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

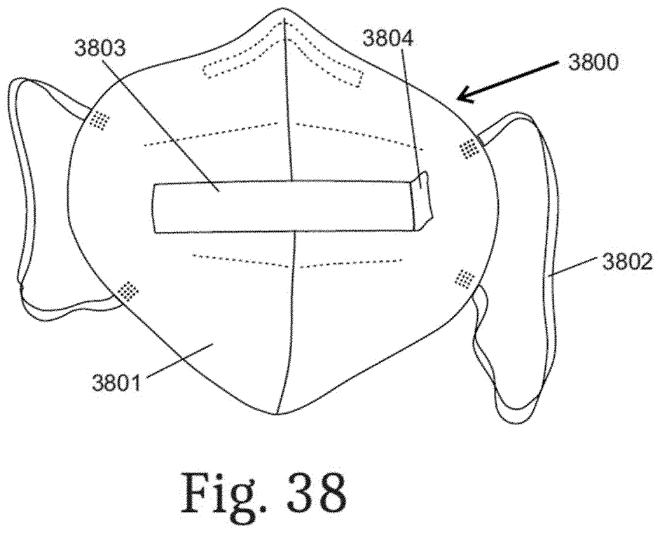

FIG. 38 shows a protective mask 3800 with an alternative configuration of a horizontal strip collection substrate 3803 removably attached to the mask, according to an exemplary embodiment of the present subject disclosure. The mask 3800 has a protective filter material with an interior face 3801 and a strap 3802 for holding the mask 3800 to the wearer's face by looping over the ears or around the back of neck and head. The mask 3800 has a collection substrate 3803 which is removably bonded to the interior face 3801 of the mask 3800 using a removable adhesive.

Collection substrate 3803 can be selected from a range of filter materials that will be known to those skilled in the art, including but not limited to: non-woven, woven, membrane, fiber, depth, electret, hydrophobic, or hydrophilic filtering materials. Further the collection substrate 3803 may be constructed with an outside cover web or a flexible or semi-rigid inside structure that is designed to provide durability. Collection substrate 3803 is positioned in a horizontal orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 3800 will generally be worn for a time period between 5 minutes and 8 hours at which point the user or another person assisting the wearer can remove the collection substrate 3803 or the wearer can go to a clinical or testing facility to have the substrate removed by a healthcare worker. To remove the substrate, mask 3800 can first be removed or the tab 3804 can be located near the exterior of the mask 3800, where it can be grasped by the worker. The tab 3804 is grasped tightly with a gloved hand, forceps or other tool and is pulled strongly, pulling the collection substrate 3803 away from the removable adhesive bond and mask 3800. During this operation, the person removing the collection substrate 3803 may grasp the mask 3800, as necessary, to hold it steady.

When wearing the mask 3800, exhaled air can pass by the collection substrate 3803, and if it is manufactured from a permeable material, through it also, thus capturing viral or microbial particles of interest in the process. Placement of the collection substrate 3803 near the breathing zone allows for increased collection of target particles and design of the substrate to easily fit into a vortex tube enables more efficient elution into a small sample volume. In one design configuration, tab 3805 is located near the outside edge of mask 3800 so it is easy for the health care worker or other person to remove the collection substrate 3803. Because collection substrate 3803 can be easily removed from outside the mask, the potential for contamination of the health care worker or cross contamination of other samples is minimized. After removal of the collection substrate 3803, target may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

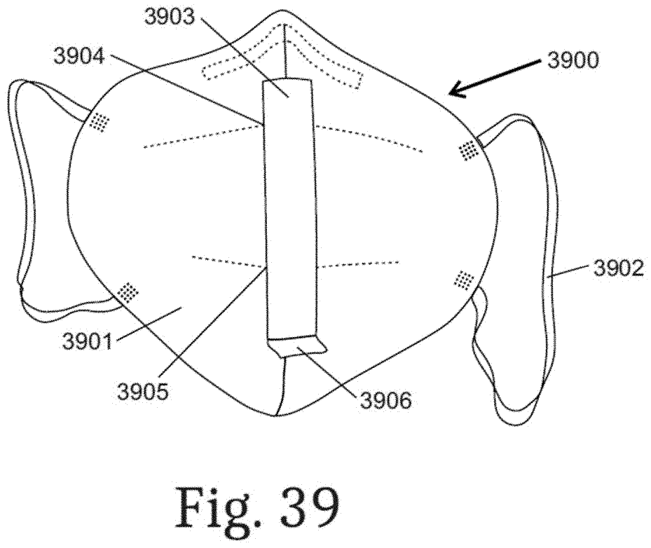

FIG. 39 shows a protective mask with a vertical strip collection substrate with a welded attachment to the mask, according to an exemplary embodiment of the present subject disclosure.

FIG. 39 shows a protective mask 3900 with vertical strip collection substrate 3903 with breakable weld line 3904 and 3905 attachments to the mask, according to an exemplary embodiment of the present subject disclosure. The mask 3900 has an interior face 3901 and a strap 3902 for holding the mask 3900 to the wearer's face by looping over the ears or around the back of neck and head. The mask 3900 has a vertical strip collection substrate 3903 which is removably bonded using a weld line 3904 and a weld line 3905 to the interior face 3901 of the mask 3900.

Collection substrate 3903 can be selected from a range of filter materials that will be known to those skilled in the art, including but not limited to: non-woven, woven, membrane, fiber, depth, electret, hydrophobic, or hydrophilic filtering materials. Further the collection substrate 3903 may be constructed with an outside cover web or a flexible or semi-rigid inside structure that is designed to provide durability. Collection substrate 3903 is positioned in a vertical orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 3900 will generally be worn for a time period between 5 minutes and 8 hours at which point the user or another person assisting the wearer can remove the collection substrate 3903 or the wearer can go to a clinical or testing facility to have the substrate removed by a healthcare worker. To remove the substrate, mask 3900 can first be removed or the tab 3905 can be located near the exterior of the mask 3900, where it can be grasped by the worker. The tab 3906 is grasped tightly with a gloved hand, forceps or other tool and is pulled strongly, to break the weld lines 3904 and 3905, and the collection substrate 3903 is pulled away from mask 3900. During this operation, the person removing the collection substrate 3903 may grasp the mask 3900, as necessary, to hold it steady.

When wearing the mask 3900, exhaled air can pass by the collection substrate 3903, and if it is manufactured from a permeable material, through it also, thus capturing viral or microbial particles of interest in the process. Placement of the collection substrate 3903 near the breathing zone allows for increased collection of target particles and design of the substrate to easily fit into a vortex tube enables more efficient elution into a small sample volume. In one design configuration, tab 3906 is located near the outside edge of mask 3900 so it is easy for the health care worker or other person to remove the collection substrate 3903. Because collection substrate 3903 can be easily removed from outside the mask, the potential for contamination of the health care worker or cross contamination of other samples is minimized. After removal of the collection substrate 3903, target may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

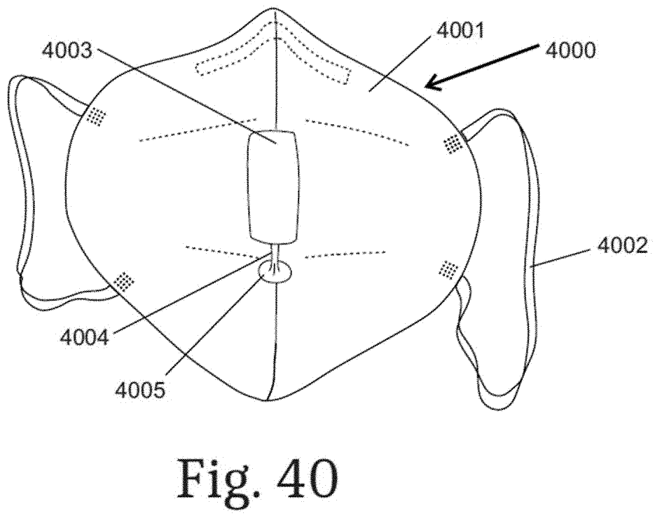

FIG. 40 shows a protective mask 4000 with a pull-through, vertical strip collection substrate 4003, according to an exemplary embodiment of the present subject disclosure. The mask 4000 has a protective filter material with an interior face 4001 and includes a strap 4002 for holding the mask 4000 to the wearer's face by looping over the ears or around the back of neck and head. The mask 4000 has a collection substrate 4003 which is removably bonded to the interior face 4001 of the mask 4000 in a vertical orientation. The collection substrate 4003 is bonded to plastic stick 4004 with disk 4005 covering an opening through the mask.

Collection substrate 4003 can be selected from a range of filter materials that will be known to those skilled in the art, including but not limited to: non-woven, woven, membrane, fiber, depth, electret, hydrophobic, or hydrophilic filtering materials. Further the collection substrate 4003 may be constructed with an outside cover web or a flexible or semi-rigid inside structure that is designed to provide durability. Collection substrate 4003 is positioned in a vertical orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

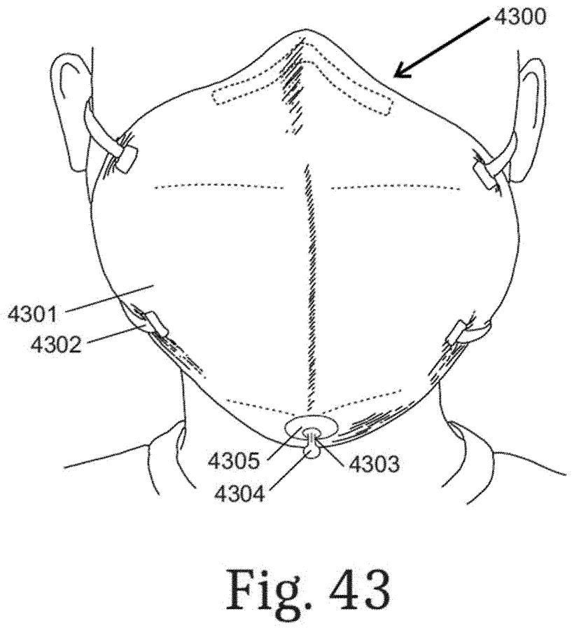

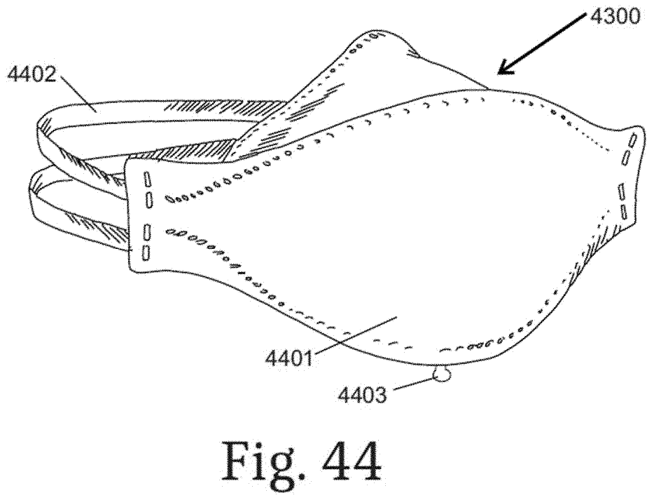

The mask 4000 will generally be worn for a time period between 5 minutes and 8 hours at which point the user or another person assisting the wearer can remove the collection substrate 4003 or the wearer can go to a clinical or testing facility to have the substrate removed by a healthcare worker. To remove the collection substrate 4003, plastic stick 4004, which extends past disk 4005 and through an opening in the mask 4000, as shown in FIGS. 43 and 44, is grasped tightly with a gloved hand, forceps or other tool and is pulled strongly, to break the removable adhesive, and the collection substrate 4003 is pulled out through the opening. During this operation, the person removing the collection substrate 4003 may grasp the mask 4000, as necessary, to hold it steady. After removal, a small adhesive sticker can be placed over opening 4004 to eliminate air leakage through the opening.

Thus, when wearing the mask 4000, exhaled air can pass by the collection substrate 4003. Placement of the collection substrate 4003 near the breathing zone allows for increased collection of target particles and design of the substrate to easily fit into a vortex tube enables more efficient elution into a small sample volume. After removal of the collection substrate 4003, target particles may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

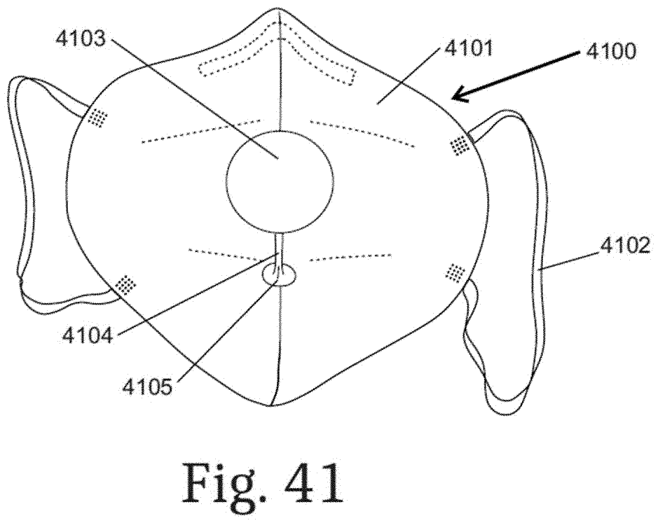

FIG. 41 shows a protective mask 4100 with a pull-through, flat disk-shaped collection substrate 4103, according to an exemplary embodiment of the present subject disclosure. The mask 4100 has a protective filter material with an interior face 4101 and includes a strap 4102 for holding the mask 4100 to the wearer's face by looping over the ears or around the back of neck and head. The mask 4100 has a flat disk-shaped collection substrate 4103 which is removably bonded to the interior face 4101 of the mask 4100 in a vertical orientation. The collection substrate 4103 is bonded to plastic stick 4104 with disk 4105 covering an opening through the mask.

Collection substrate 4103 can be selected from a range of filter materials that will be known to those skilled in the art, including but not limited to: non-woven, woven, membrane, fiber, depth, electret, hydrophobic, or hydrophilic filtering materials. Further the collection substrate 4103 may be constructed with an outside cover web that is designed to provide durability. Collection substrate 4103 is positioned in a vertical orientation near the mask wearer's breathing zone, in close proximity to the wearer's mouth and nose. The collection substrate is of a size and shape that is easily inserted directly or after rotating slightly into a vortex or centrifugation sample tube between 100 microliter and 100 mL in internal volume.

The mask 4100 will generally be worn for a time period between 5 minutes and 8 hours at which point the user or another person assisting the wearer can remove the collection substrate 4103 or the wearer can go to a clinical or testing facility to have the substrate removed by a healthcare worker. To remove the collection substrate 4103, plastic stick 4104, which extends past disk 4105 and through an opening in the mask 4100, as shown in FIGS. 43 and 44, is grasped tightly with a gloved hand, forceps or other tool and is pulled strongly, to break the removable adhesive, and the collection substrate 4103 is pulled out through the opening. During this operation, the person removing the collection substrate 4103 may grasp the mask 4100, as necessary, to hold it steady. After removal, a small adhesive sticker can be placed over opening 4104 to eliminate air leakage through the opening.

Thus, when wearing the mask 4100, exhaled air can pass by the collection substrate 4103. Placement of the collection substrate 4103 near the breathing zone allows for increased collection of target particles and design of the substrate to easily fit into a vortex tube enables more efficient elution into a small sample volume. After removal of the collection substrate 4103, target particles may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

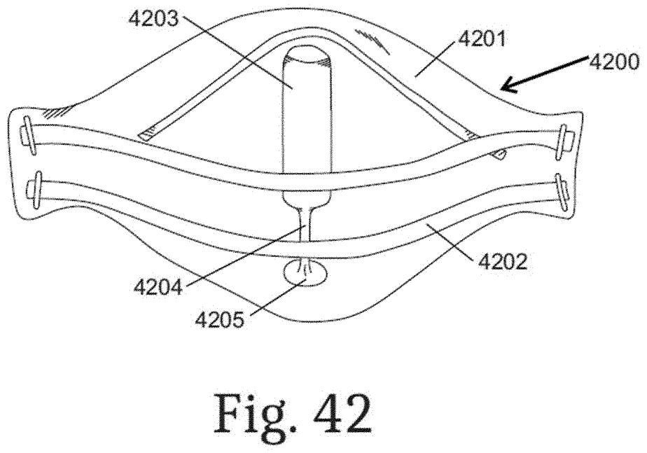

FIG. 42 shows a protective mask 4200 with a pull-through, cylindrical collection substrate 4203, according to an exemplary embodiment of the present subject disclosure. The mask 4200 has a protective filter material with an interior face 4201 and includes a strap 4202 for holding the mask 4200 to the wearer's face by looping over the ears or around the back of neck and head. The mask 4200 has a cylindrical collection substrate 4203 which is removably bonded to the interior face 4201 of the mask 4200 in a vertical orientation. The collection substrate 4203 is bonded to plastic stick 4204 with disk 4205 covering an opening through the mask.

Collection substrate 4203 can be selected from a range of filter materials that will be known to those skilled in the art, including but not limited to: non-woven, woven, membrane, fiber, depth, electret, hydrophobic, or hydrophilic filtering materials. Further the collection substrate 4203 may be constructed with an outside cover web or a flexible or semi-rigid inside structure that is designed to provide durability. Collection substrate 4203 is positioned in a vertical orientation near the mask wearer's breathing zone, in close proximity to the wearer's mouth and nose. The collection substrate is of a size and shape that is easily inserted into a vortex or centrifugation sample tube between 100 microliter and 100 mL in internal volume.

The mask 4200 will generally be worn for a time period between 5 minutes and 8 hours at which point the user or another person assisting the wearer can remove the collection substrate 4203 or the wearer can go to a clinical or testing facility to have the substrate removed by a healthcare worker. To remove the collection substrate 4203, plastic stick 4204, which extends past disk 4205 and through an opening in the mask 4200, as shown in FIGS. 43 and 44, is grasped tightly with a gloved hand, forceps or other tool and is pulled strongly, to break the removable adhesive, and the collection substrate 4203 is pulled out through the opening. During this operation, the person removing the collection substrate 4203 may grasp the mask 4200, as necessary, to hold it steady. After removal, a small adhesive sticker can be placed over opening 4204 to eliminate air leakage through the opening.

Thus, when wearing the mask 4200, exhaled air can pass by the collection substrate 4203. Placement of the collection substrate 4203 near the breathing zone allows for increased collection of target particles and design of the substrate to easily fit into a vortex tube enables more efficient elution into a small sample volume. After removal of the collection substrate 4203, target particles may be extracted by placing it into a sample tube or other container and vortexing, shaking, sonicating, rocking, or performing wet foam elution, or other methods that will be well known to those skilled in the art.

Following extraction, the liquid sample may be analyzed using techniques that will be well known to those skilled in the art, including microbiological methods such as: polymerase chain reaction, sequencing, antigen assays, biochemical assays, molecular detection methods, rapid microbiological detection methods, mass spectrometry, chemical, cytometry, flow cytometry, or growth-based microbiology methods to detect the particles. Additionally, chemical analysis techniques may be used on the extracted sample to detect and identify particles of biological or non-biological nature.

FIG. 43 shows a fold-flat protective mask 4301 with a pull-through, collection substrate on a plastic stick with handle 4304, according to an exemplary embodiment of the present subject disclosure. The mask 4300 has a protective filter material with an exterior face 4301 and includes a strap 4302 for holding the mask 4300 to the wearer's face by looping over the ears or around the back of neck and head. The mask 4300 has an internal collection substrate like those shown in FIGS. 40, 41, and 42 which is removably bonded to the interior face of the mask 4300 in a vertical orientation. The internal collection substrate is bonded to plastic stick with handle 4304 which extends out of the mask through opening 4303 with adhesive or welded disk 4305 covering an opening through the mask.

The mask 4300 will generally be worn for a time period between 5 minutes and 8 hours at which point the user or another person assisting the wearer can remove the collection substrate or the wearer can go to a clinical or testing facility to have the substrate removed by a healthcare worker. To remove the collection substrate, plastic stick with handle 4304, which extends through opening 4303 is grasped tightly with a gloved hand and is pulled strongly to break the removable adhesive or welded disk 4305 seal, and the collection substrate is pulled out through the opening. During this operation, the person removing the collection substrate may grasp the mask 4300, as necessary, to hold it steady. After removal, a small adhesive sticker can be placed over opening 4303 to eliminate air leakage through the opening.

FIG. 44 shows an alternative configuration of a fold-flat protective mask 4401 with a pull-through, collection substrate on a plastic stick with handle 4404, according to an exemplary embodiment of the present subject disclosure. The mask 4400 has a protective filter material with an exterior face 4401 and includes a strap 4402 for holding the mask 4400 to the wearer's face by looping over the ears or around the back of neck and head. The mask 4400 has an internal collection substrate like those shown in FIGS. 40, 41, and 42 which is removably bonded to the interior face of the mask 4400 in a vertical orientation. The internal collection substrate is bonded to plastic stick with handle 4404 which extends out of the mask through opening 4403 with adhesive or welded disk 4405 covering an opening through the mask.

The mask 4400 will generally be worn for a time period between 5 minutes and 8 hours at which point the user or another person assisting the wearer can remove the collection substrate or the wearer can go to a clinical or testing facility to have the substrate removed by a healthcare worker. To remove the collection substrate, plastic stick with handle 4404, which extends through opening 4403 is grasped tightly with a gloved hand and is pulled strongly to break the removable adhesive or welded disk 4405 seal, and the collection substrate is pulled out through the opening. During this operation, the person removing the collection substrate may grasp the mask 4400, as necessary, to hold it steady. After removal, a small adhesive sticker can be placed over opening 4403 to eliminate air leakage through the opening.

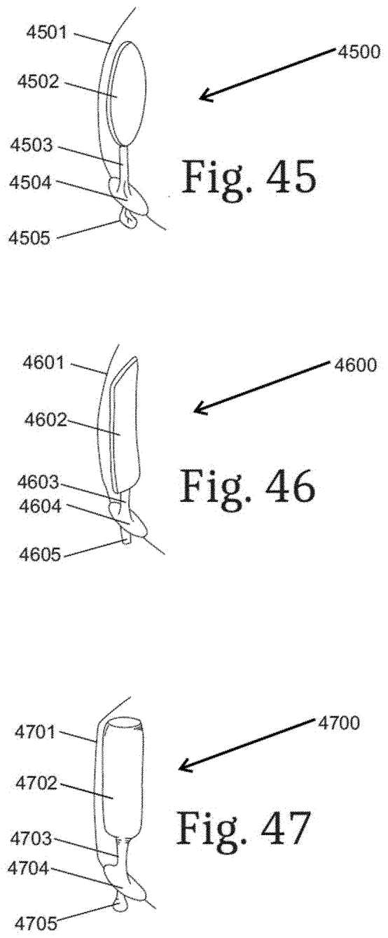

FIG. 45 shows a disk-shaped swab-style collection device for masks 4500 with a pull-through, flat disk-shaped collection substrate 4502 on a plastic (or other material) stick 4503, according to an exemplary embodiment of the present subject disclosure. The flat disk-shaped collection substrate 4502 is installed into mask 4501. The flat disk-shaped collection substrate 4103 may be removably bonded to the interior face of mask 4501 in a vertical orientation. The flat disk-shaped collection substrate 4502 is bonded to plastic stick 4503 with disk 4504 and handle 4505. Disk 4504 covers an opening through the mask that the stick extends through. This configuration is like the collection substrate in FIG. 41.

Flat disk-shaped collection substrate 4502 can be selected from a range of filter materials that will be known to those skilled in the art, including but not limited to: non-woven, woven, membrane, fiber, depth, electret, hydrophobic, or hydrophilic filtering materials. Further the flat disk-shaped collection substrate 4502 may be constructed with an outside cover web that is designed to provide durability.

FIG. 46 shows a strip swab-style collection device for masks 4600 with a pull-through, strip collection substrate 4602 on a plastic (or other material) stick 4603, according to an exemplary embodiment of the present subject disclosure. The strip collection substrate 4602 is installed into mask 4601. The strip collection substrate 4103 may be removably bonded to the interior face of mask 4601 in a vertical orientation. The strip collection substrate 4602 is bonded to plastic stick 4603 with disk 4604 and handle 4605. Disk 4604 covers an opening through the mask that the stick extends through. This configuration is like the collection substrate in FIG. 40.

Strip collection substrate 4602 can be selected from a range of filter materials that will be known to those skilled in the art, including but not limited to: non-woven, woven, membrane, fiber, depth, electret, hydrophobic, or hydrophilic filtering materials. Further the strip collection substrate 4602 may be constructed with an outside cover web that is designed to provide durability.

FIG. 47 shows a cylindrical swab-style collection device for masks 4600 with a pull-through, cylindrical collection substrate 4702 on a plastic (or other material) stick 4703, according to an exemplary embodiment of the present subject disclosure. The cylindrical collection substrate 4702 is installed into mask 4701. The cylindrical collection substrate 4103 may be removably bonded to the interior face of mask 4701 in a vertical orientation. The cylindrical collection substrate 4702 is bonded to plastic stick 4703 with disk 4704 and handle 4705. Disk 4704 covers an opening through the mask that the stick extends through. This configuration is like the collection substrate in FIG. 42.

Cylindrical collection substrate 4702 can be selected from a range of filter materials that will be known to those skilled in the art, including but not limited to: non-woven, woven, membrane, fiber, depth, electret, hydrophobic, or hydrophilic filtering materials. Further the cylindrical collection substrate 4702 may be constructed with an outside cover web that is designed to provide durability.

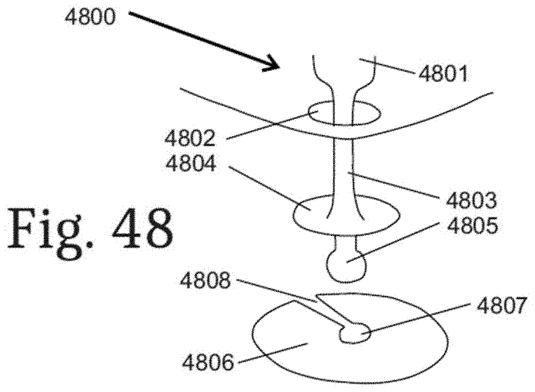

FIG. 48 shows plastic adhesive sticker seal system 4800 for a pull-through, collection substrate 4801 on a plastic (or other material) stick 4803. A collection substrate 4801 on stick 4804, like those shown in FIG. 45 through 47, extends through opening 4802 in a protective mask. Disk 4804 covers opening 4802 and is covered by adhesive sticker with opening 4807 and slit 4808. This assembly allows collection substrate 4801 to be mounted inside of a protective mask with a handle 4805 outside of said mask. The handle 4805 allows a health care worker or other person to grasp the handle with a gloved hand and pull the collection substrate 4801 out of the mask, without requiring the wearer to remove the mask. After pulling the collection substrate out of the mask the worker can seal the opening 4802 with an adhesive sticker.

FIG. 49 provides an example table of how using source and receptor filters in series allows for data to be used to determine if the detected particles originated from the wearer or from the environment around the wearer. Assuming a 77.7% efficiency for each filter layer (e.g., source/interior and receptor/exterior), and in the case of an infected wearer that is shedding virus, the interior source collection filter will collect a minimum of approximately 4.5 times more target particles than the exterior receptor collection filter. This difference is sufficient to allow for a determination as to whether target particles originated from an infected wearer or a contaminated environment. Further, as the efficiency of each layer is increased the ratio of target particles collected by each filter increases. As an example, two 90% efficiency filters will provide a minimum of approximately 10 times more particles on source collection filter if the wearer is shedding virus or microbes. Finally, these estimated ratios are most likely lower than they will be in actual use cases because not all particles containing target material are 0.3 μm in diameter, but are rather distributed over a large particle size range, and, in general, particles larger than and smaller than 0.3 μm in diameter are captured with higher efficiency which will lead to larger ratios.

FIG. 50 provides an example of how using source and receptor filters in series allows for data to be used to determine if the detected particles originated from the wearer or from the environment around the wearer. Assuming a 77.7% efficiency for each filter layer (e.g. source/interior and receptor/exterior), and in the case of a contaminated environment or a person in close proximity shedding virus or microbes, the exterior receptor collection filter will collect a minimum of approximately 4.5 times more target particles than the interior source collection filter. This difference is sufficient to allow for a determination as to whether target particles originated from an infected wearer or a contaminated environment. Further, as the efficiency of each layer is increased the ratio of target particles collected by each filter increases. As an example, two 90% efficiency filters will provide a minimum of approximately 10 times more particles on receptor collection filter if the wearer is in a contaminated environment or a person in close proximity is shedding virus or microbes. Finally, these estimated ratios are most likely lower than they will be in actual use cases because not all particles containing target material are 0.3 μm in diameter, but are rather distributed over a large particle size range, and, in general, particles larger than and smaller than 0.3 μm in diameter are captured with higher efficiency which will lead to larger ratios.

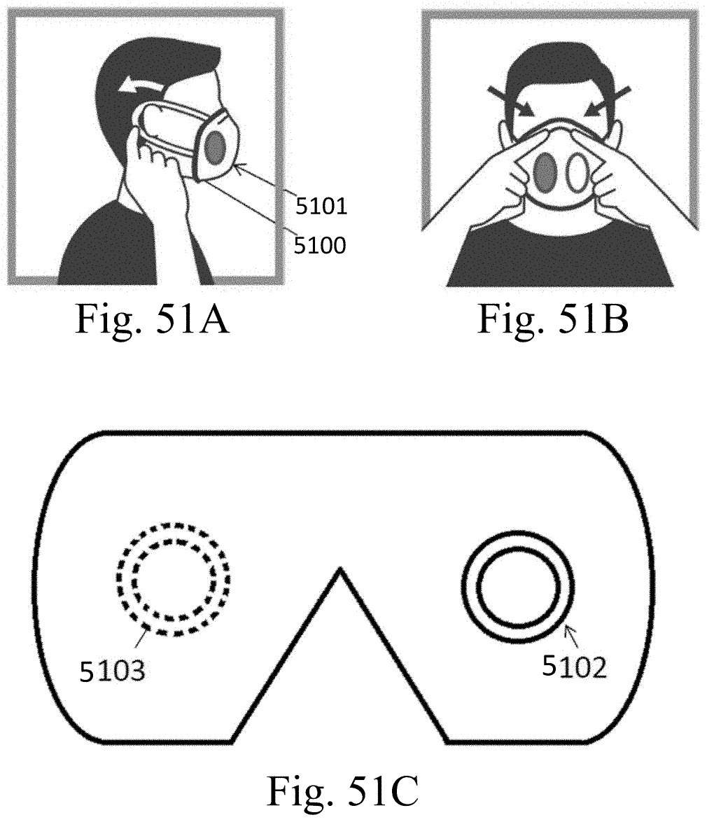

FIGS. 51A-51B show a source/receptor mask or respirator (S/R Mask) 5100, according to an exemplary embodiment of the present subject disclosure. FIG. 51A shows a S/R mask 5100 which includes an exterior portion 5101. A first exterior (receptor) filter 5102, may have an optional attachment ring to help secure/attach the filter. A second interior (source) filter 5103, may also have an optional attachment ring to help secure/attach the filter. The right/left side as well as the outer/inner side positions of the source and receptor filters may be interchanged or they may both be on the same left or right side. In any case, one filter (“receptor filter”) should be positioned to be exposed to the outside mask air, and the other filter positioned to be exposed to the inside mask air (“source filter”).

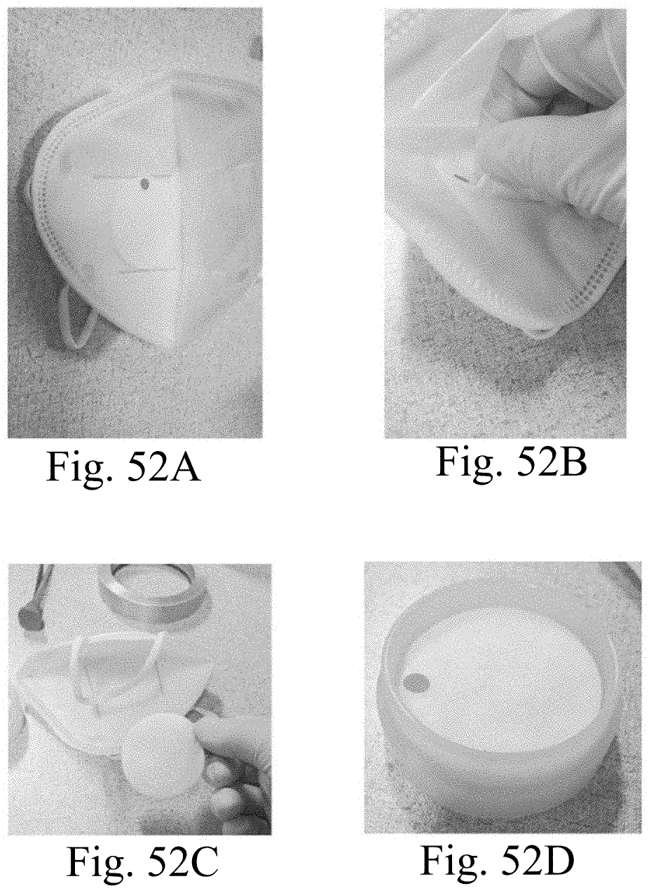

In one exemplary embodiment, the present subject disclosure is a device constructed as a folding KN95 mask with adhesive tape circles applied. One on the inside, and one, opposite, on the outside of the mask. The tape is stickier on the mask side than on the filter side, allowing the filter to adhere adequately during use, yet be peeled off without damage for elution and subsequent analysis. The filter is constructed of a low pressure drop electret material (approximately 0.1″ H2O to 1.0″ H2O, and preferably approximately 0.5″ H2O for a 47 mm diameter effective area). FIGS. 52A-52D shows a way to remove the two filters from a mask.

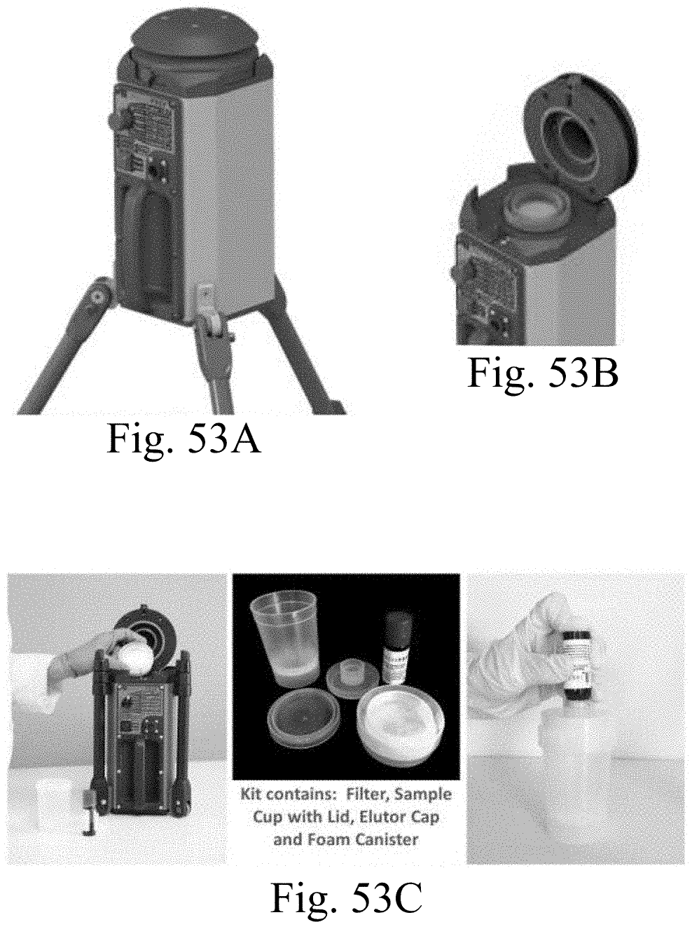

FIGS. 53A-53C show an air sample and filter elution system, and kit, based on InnovaPrep's Bob cat and Wet-Foam Elution methodologies, which are incorporated by reference herein in their entirety.

The filter material itself can be removed (see steps of FIG. 52A-52D) and be eluted in multiple ways including use of Wet Foam Elution, swirling and or shaking in an aqueous elution fluid, or bead beating. Wet foam Elution can be performed using the InnovaPrep Bobcat (U.S. Pat. Nos. 8,584,536; 9,534,989; etc.) aerosol sampler filter elution kit or a similar kit designed specifically for use with the mask filters or with an automated or semi-automated system for quickly delivering a wet foam that passes through the filter, recovering particles from the filter in the process. Swirling, shaking, and bead beating elutions can be performed using standard, laboratory instrumentation that is well known to those skilled in the art, such as vortexers, laboratory shakers, and laboratory bead beaters.

In one embodiment, the filter, once removed, is placed in a filter holder ring and flushed with a liquid such as is performed using the InnovaPrep Bobcat aerosol sampler filter elution kit. This liquid can be flat or can be a foam made of a soluble gas dissolved in a weakly detergent buffer solution, such that it more efficiently elutes the particles, yet collapses rapidly into a flat liquid for sample preparation and/or analysis (based on InnovaPrep's flat filter methodologies, as described in more detail in the patents cited above). This foam elution process—known as Wet Foam Elution—can be used to efficiently recover particles from the filter into smaller volumes than the other methods described herein.

Samples may be pooled and eluted simultaneously in this device or a bigger device functioning similarly.

Samples may be pooled for analysis with or without sample concentration prior to analysis, depending on the sensitivity of the analytical method and the desired detection limit.

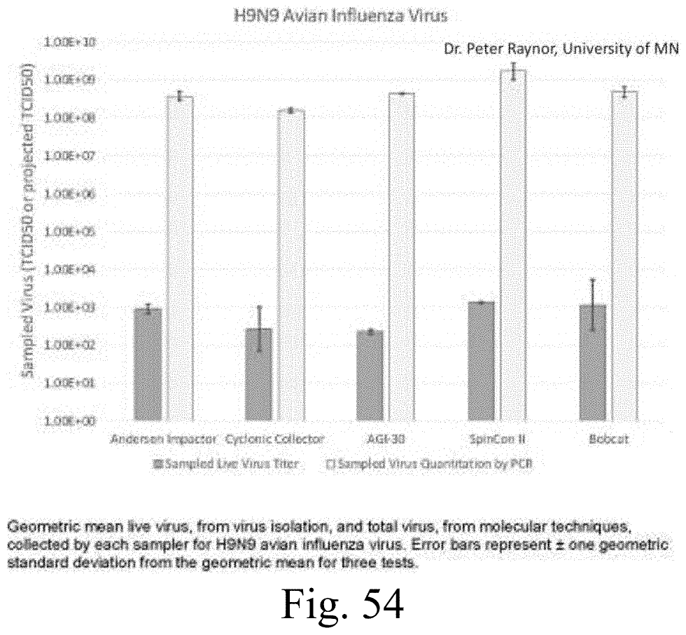

FIG. 54 shows virus presence using various filtration techniques.

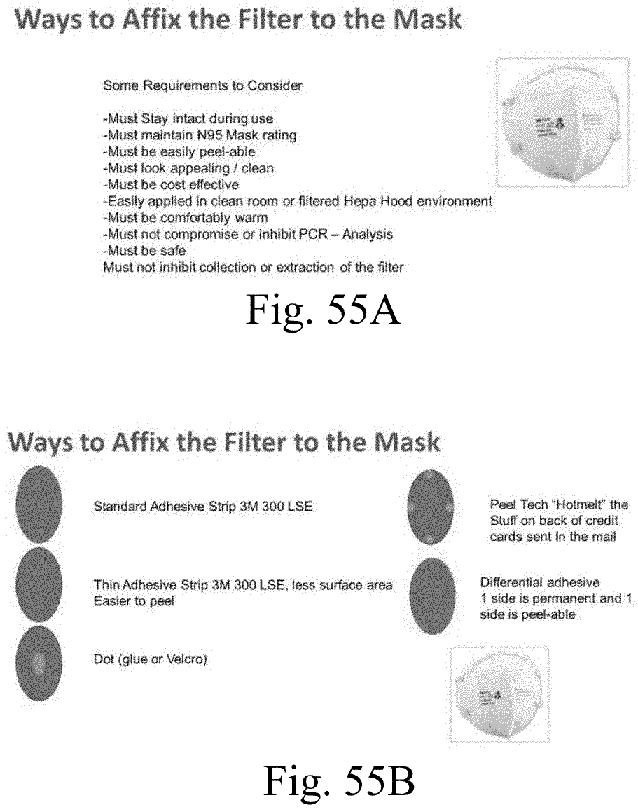

FIGS. 55A and 55B show requirements to consider for a mask, and ways to affix a filter to a mask, respectively, according to an exemplary embodiment of the present subject disclosure.

In another embodiment, each filter can be placed into separate sample tubes with a liquid to remove the sample from the filter into the fluid via shaking, vortexing, or bead beating. This fluid can be water, or water with a surfactant added, or water with multiple additives (such as viral transport media) for removing the sample and stabilizing it for transport.

In another embodiment, each filter can be enriched or cultured, by placing the filter into a liquid growth media or onto a solid growth media that is selected for enrichment of the target microorganism.

In another embodiment, a single filter is attached to the outside of the mask for receptor sampling only; with no internal filter for source sampling. This approach may be used for non-microbial sampling, and for instances in which the microorganism is environmental and not human transmissible. In this case the analysis method may be microorganism based or chemical based, dependent on the target particle characteristics.

Alternatively, in another embodiment, a single filter is attached to the inside of the mask for source sampling only; with no external filter for receptor sampling. This approach may be used for instances in which humans/animals are tested before entry into a given area to determine whether they would be infective.

In another embodiment, check valves may be integrated into the respirator or mask such that upon breathing in the user breaths through one “receptor” filter and upon breathing out they breathe through a second “source” filter. See, for example, FIG. 56B.

Another exemplary embodiment is in the shape of a tube having a receptor filter and a source filter, and operating essentially as described herein. See FIGS. 56A-56B. This embodiment has been built and strategically sized for easy breathing and effective elution.

In the case of pooling samples, for example from a family or work pod, receptor samples are pooled together and source filters are pooled together in a separate pool. To avoid loss of information, samples can be tagged with molecular barcodes prior to pooling. Pooling can be performed prior to elution, but eluting multiple samples together, or the liquid samples may be pooled after elution. Pooling of liquid samples, after elution, has the distinct advantage of enabling an archive sample to be collected from each filter sample prior to pooling. In the event of a positive result for the target, the laboratory can then analyze individual samples from the pool and determine which single or multiple mask samples within the pooled was the cause of the positive pool result.

Pooling of samples comes with the inherent risk of a reduced method sensitivity. When pooling a single positive filter pooled with many negative filters can result in a negative result for the pool due to dilution of the positive sample. However, using sample concentration to concentrate the pooled sample volume to a smaller volume than the original single sample elution volume can actually provide improved sensitivity over analyzing the single positive sample without sample concentration.

In the case of sample concentration, the InnovaPrep Concentrating Pipette systems, which are incorporated by references herein in their entirety into this disclosure, or other method may be used to concentrate the particles from the mask filters prior to analysis. Such methods include, but are not limited to, re-filtration, centrifugation, chemical precipitation, or affinity-based concentration technologies.

A concentrated sample may be further concentrated prior to analysis by immunomagnetic separation, electrophoretic or dielelectrophoretic separation techniques, or other microfluidic concentration techniques. In many instances these techniques are useful but are in general not possible with larger volumes or are prohibitively costly or slow when performed on large volumes. By rapidly performing an initial concentration with the Concentrating Pipette, the sample volume is reduced to a volume that is more readily handled with these techniques.

It is further possible to apply additional sample preparation techniques to the concentrated sample once dispensed. Additional sample preparation techniques that may be applied include various methods of cell lysis, washing steps, inhibitor or interferent removal techniques, and labeling steps. Reduction of the sample volume prior to performing these techniques routinely improves the speed and efficiency, while reducing the cost of performing these techniques.

Analysis of the concentrated sample may be performed with any number of commonly used traditional analytical or microbiological analysis methods or rapid analysis techniques including rapid microbiological techniques. Analytical techniques of special interest include conventional methods of plating and enumeration, most probable number, immunoassay methods, polymerase chain reaction (PCR), sequencing, electrochemical, microarray, flow cytometry, biosensors, lab-on-a-chip, and rapid growth based detection technologies to name a few.

Samples may be analyzed in search of bioterrorism agents, or in the interest of public health and safety, especially where a sample may contain target agent(s) that are thought to be a threat to the health of humans, animals or plants, causing societal disruption and economic harm. Agricultural products and livestock environments may also be evaluated by the instrumentalities herein disclosed.

These types of sampling and analysis are advantageously performed for the fields of pandemic preparedness and response, public health, health care, homeland security, corporate security, and military force protection. Additional fields of use include medical research and diagnostics.

The foregoing instrumentalities have significant utility in medical, environmental, or security applications. In exemplary embodiments, concentration in the manner described facilitates aerosol sampling for pathogens such as MERS, SARS, SARS-CoV-2, or bioterrorism threat agents that can withstand being placed in a liquid sample for analysis. A list of such pathogens may be provided, for example, as recognized by the Center for Disease Control (CDC). These organisms may be studied using conventional techniques that are facilitated by the concentration of samples as described above. It should be noted that many of the below could be inhaled from the environment and a few could be exhaled by an infected individual. Table 1 provides a list of CDC Category A and B Bioterrorism Agents list. Table 2 provides a list of Secondary Potential Biological Threat Agents. Table 3 provides a list of Physical Sizes of some Agents and Surrogates.

TABLE 1
CDC CATEGORY A AND B BIOTERRORISM AGENTS LIST
CATEGORY A
Anthrax (Bacillus anthracis)
Botulism (Clostridium botulinum toxin)
Plague (Yersinia pestis)
Smallpox (Variola major)
Tularemia (Francisella tularensis)
Viral hemorrhagic fevers (filoviruses [e.g., Ebola, Marburg]
and arenaviruses [e.g., Lassa, Machupo])
CATEGORY B
Brucellosis (Brucella species)
Epsilon toxin of Clostridium perfringens
Food safety threats (e.g., Salmonella species, Escherichia coli
O157:H7, Shigella)
Glanders (Burkholderia mallei)
Melioidosis (Burkholderia pseudomallei)
Psittacosis (Chlamydia psittaci)
Q fever (Coxiella burnetii)
Ricin toxin from Ricinus communis (castor beans)
Staphylococcal enterotoxin B
Typhus fever (Rickettsia prowazekii)
Viral encephalitis (alphaviruses [e.g., Venezuelan equine encephalitis,
Eastern equine encephalitis, Western equine encephalitis])
Water safety threats (e.g., Vibrio cholerae, Cryptosporidium parvum)

TABLE 2
SECONDARY POTENTIAL BIOLOGICAL THREAT AGENTS

| | |
|---|---|
| Viri/prions | Histoplasma capsulatum |
| Flaviviruses (Yellow fever virus, West | Cryptococcus neoformans |
| Nile virus, Dengue, Japanese | Aspergillus niger |
| Encephalitis, TBE, etc.) | Pathogenic fungi |
| Hepatitis (A, B, C) | Acremomium spp. |
| Prions (CJD, BSE, CWD) | Alternaria alternate |
| Alphaviruses (VEE, EEE, WEE) | Apophysomyces elegans |
| Nipah virus | Aspergillus terreus |
| Rabies virus | Bipolaris spp. |
| Rhinovirus (could be modified?) | Bipolaris spicifera |
| Polioviruses | Blastoschizomyces capitatus |
| Hantaviruses | Candida krusei |
| Filoviruses (Ebola, Marburg, Lassa) | Candida lusitaniae |
| Bacilli | Cladophialophora bantiana |
| Mycobacterium tuberculosis, | Cunnihamella berholletiae |
| drug resistant | Curvularia lunata |
| Mycobacteria other than TB, | Exserohilum rostratum |
| like C. leprae | Fusarium moniliforme |
| Streptococcus pneumoniae | Fusarium solani |
| Streptococcus pyogenes | Hansenula anomala |
| Streptococcus aureus | Lasiodilodia theobromae |
| Clostridium tetani | Malassezia furfur |
| Clostridium difficile | Paecilomyces lilacinus |
| Bacillus cereus | Paecilomyces bariotii |
| Coxiella brunette (Q fever) | Penicillium marneffei |
| Francisella tularensis | Phialemonium curvatum |
| Borrelia recurrentis | Phialophora parasitica |
| Rickettsia rickettsii | Phialophora richardsiae |
| R. prowazekii | Ramichloridium spp. |
| Shigella sonnei | Rhizomucor pusillus |
| Bartonella henselae | Rhizopus rhizopodiformus |
| Yersinia enterolitica | Rhodotorula rubra |
| Yersinia pseudotuberculosis | Sacchromyces cerevisiae |
| Neisseria meningitidis | Scedosporium prolificans |
| Legionella pneumophila | Trichosporon beigelii |
| Burkholderia pseudomallei | (T. asahii) |
| Pasturella multocida | Wangiella dermatitidis |
| Other Pathogenic Microorganisms | |
| Cryptosporidium parvum | |

TABLE 3
PHYSICAL SIZES OF SOME AGENTS AND SURROGATES

| TARGET | PHYSICAL SIZE |
|---|---|
| Bacillus thuringiensis endospore | approximately 1 μm |
| Bacillus anthracis endospore | approximately 1 μm |
| Yersinia pestis | Gram negative rod-ovoid 0.5-0.8 μm in |
| | width and 1-3 μm in length |
| Yersinia rohdei | approximately 1 μm |
| Venezuelan Equine Encephalitis | 80 nm (0.07 μm) |
| Gamma-killed MS2 | 2 mD or about 25 nm (0.025 μm) (but |
| | will pass through a 400 kD pore size |
| | but is retained by a 100 kD pore size |
| | Wick and McCubbin - ECBC) |
| Ovalbumin | 45 kD or 6 nm (0.006 μm) |
| Botulinum Toxoid A | 160 to 900 kD or 10 nm to 80 nm (0.01 |
| | μm to 0.07 μm)(Normally published as |
| | 160 kD however some publications |
| | state that toxoid A can be released |
| | as complexes comprised of the 160 |
| | kD toxin protein along with associated |
| | non-toxin proteins and can therefore |
| | be released in 900 kD, 600 kD, and |
| | 400 kD forms. |
| DNA | 1000 Bp or 600 kD up to 15,000 Bp |
| | or 9 mD |

The add-on filters for the device shown are depth filters with high efficiency across a wide range of all respirable particles >0 microns in diameter. A low resistance (e.g., pressure drop) filter is preferred that has lower resistance (e.g., pressure drop) or resistance to flow than the mask material itself. An example of such a filter is a 3M T90+ filter. The T90+ is only one example of a suitable electret filter, which captures ultrafine particles primarily via electrostatic forces and larger particles by interception with the fibers of the filter. Such filters have very low resistance (e.g., pressure drops). A circular 47 mm filter made of T90+ material will have a resistance (e.g., pressure drop) of approximately 0.35″ water column. Mask material such as that used in the Covaflu HDK-95 mask has a resistance (e.g., pressure drop) of approximately 0.60″ water column per 47 mm diameter circle. Many types and varieties of such filters and masks are available and may be suitable. For example, non-woven polymer fiber filters (including electret filters), glass fiber filters, felt, cotton, and paper filters of suitable specification are available. Further, as can be appreciated by someone skilled in the art, novel membranes and filters, and membranes and filters other than those mentioned here, may serve the purpose of retaining certain particles of interest and may provide a reliable filter and mask combination.

Moreover, although a collection of viruses is disclosed, any of the disclosed embodiments may be used to concentrate bacterial pathogens in exemplary embodiments, if that organism is shed into the breath of infected individuals or may be present in the air surrounding a person wearing a S/R mask.

Finally, the exemplary embodiments of the masks are shown primarily for use on humans, but as appreciated by one having ordinary skill in the art, the same features and designs may be incorporated into a mask designed and fitted for particular animals to enable the sampling of particles exhaled by the animal, and inhaled from the environment in which the animal is located.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A respiratory mask device, comprising;
a layer of protective filter material for covering the mouth and the nose of a wearer;
a collection filter assembly for capture of airborne droplets, particles, and/or aerosols for analysis and detection;
at least one opening within the filter material through which the collection filter assembly is mounted on an exterior side thereof;
wherein the collection filter assembly is removable from an exterior side of the mask when it is worn by a wearer;
wherein breathing by the wearer forces air to pass through the collection filter assembly and airborne particles to be captured onto all or a portion of the collection filter assembly;
wherein at least a portion of the collection filter assembly is removable to allow for extraction of the captured particles into a liquid prior to analysis and detection.

2. The device in claim 1, wherein the protective material comprises any type of respirator or respiratory protective mask constructed of a porous material.

3. The device in claim 1, wherein the protective material provides respiratory protection to both the wearer and to other individuals in proximity.

4. The device in claim 1, further comprising one or more elastic bands or tie straps connected to the material and adapted to be pulled around the ears or neck to secure the material to the nose and/or the mouth.

5. The device in claim 1, wherein the protective filter material is made up of one or more layers of non-woven, woven, membrane, fiber, depth, electret, hydrophobic, hydrophilic, or cover-web filtering materials.

6. The device in claim 5, wherein all or a portion of the layers in the protective filter material are also used to form all or part of the collection filter assembly.

7. The device in claim 1, wherein the collection filter assembly is bonded to the perimeter of the opening using adhesive, glue, tape, solvent bonding, ultrasonic weld, friction weld, laser welding, thermal bonding, or heat laminating.

8. The device in claim 1 wherein the collection filter assembly is made up of one or more layers of filter frame, filter support, interior coverweb, exterior coverweb, interior collection filter, and exterior collection filter.

9. The device in claim 8, wherein the interior and exterior collection filters are made up of one or more layers of non-woven, woven, membrane, fiber, depth, electret, hydrophobic, or hydrophilic, filtering materials.

10. The device in claim 8, wherein the collection filter assembly contains one or more tabs to enable removal and separation of the layers.

11. The device in claim 8, wherein the collection filter assembly and the collection filter assembly layers are adapted to be easily separated from the respiratory mask and from each other.

12. The device in claim 8, wherein the exterior collection filter collects airborne particles primarily from the environment and the interior collection filter collects airborne particles primarily exhaled by the wearer.

13. The device in claim 8, wherein exterior collection filter and interior collection filter are sized and shaped to enable users to insert each into a vortex or centrifugation sample tube between 100 microliter and 100 mL in internal volume.

14. The device in claim 1, wherein the collection filter assembly includes an indicator device sensitive to pH or moisture changes that is used to indicate that the device has been worn for a sufficient period of time.

15. The device in claim 1, wherein the collection filter assembly resistance per surface area is less than or equal to the protective filter material resistance per surface area.

16. The device in claim 1, wherein collection filter assembly is sized and shaped to enable users to insert it into a sample tube, vortex tube, or centrifugation tube between 100 microliter and 100 mL in internal volume.

17. A method for collecting and detecting airborne droplets, particles, and/or aerosols while providing respiratory protection, comprising:
providing a well-fitted device for providing respiratory protection to both a wearing individual and other individuals in close proximity which also enables collection of constituents exhaled by the wearing individual onto a collection filter assembly, wherein the wearing individual wearing the device over the nose and mouth for a period of time between 5 minutes and 8 hours;
removing the collection filter assembly from the device;
removing a collection filter or filters from the collection filter assembly;
placing the collection filter or filters into sample tubes along with an extraction fluid;
extracting captured particles from the collection filter assembly into the extraction fluid; and
detecting the captured particles.

18. The method of claim 17, wherein the entire collection filter assembly is placed into a sample tube for extraction.

19. The method in claim 17, wherein the extracting step includes vortexing, shaking, rocking, sonication, or wet foam elution.

20. The method of claim 17, further comprising detecting the particles using one or more of polymerase chain reaction, sequencing, antigen assays, biochemical assays, molecular detection methods, rapid microbiological detection methods, mass spectrometry, chemical, cytometry, flow cytometry, or growth-based microbiology methods.
